(19) United States
(12) Patent Application Publication
(10) Pub. No.: US 2025/0019737 A1
(43) Pub. Date: Jan. 16, 2025

(54) BACTERIAL SYSTEMS FOR ANALYZING UBIQUITYLATED POLYPEPTIDES

(71) Applicant: Technology Innovation Momentum Fund (Israel) Limited Partnership, Tel-Aviv (IL)

(72) Inventor: Gali PRAG, Tel-Aviv (IL)

(21) Appl. No.: 18/891,666

(22) Filed: Sep. 20, 2024

Related U.S. Application Data
(63) Continuation of application No. 17/207,943, filed on Mar. 22, 2021.
Application No. 17/207,943 is a continuation of application No. 16/322,956, filed on Feb. 3, 2019, now Pat. No. 10,982,252.
(60) Provisional application No. 62/371,881, filed on Aug. 8, 2016.

(51) Int. Cl. C12Q 1/02; C12N 9/10; C12Q 1/48

(52) U.S. Cl. CPC C12Q 1/025; C12N 9/104; C12Q 1/48; C07K 2319/95; G01N 2333/9015; G01N 2333/9108; G01N 2500/02; G01N 2500/10

(57) ABSTRACT

Bacterial systems for analyzing ubiquitination of proteins is disclosed herein. Kits for analyzing the ubiquitination and methods for carrying out the analysis are also disclosed.

### RELATED APPLICATIONS

This application is a continuation of U.S. patent application Ser. No. 17/207,943, filed Mar. 22, 2021, which is a continuation of U.S. patent appliction No. 16/322,956, filed on Feb. 3, 2019, now U.S. Pat. No. 10,982,252, which is a National Phase of PCT Patent Application No. PCT/IL2017/050876 having International Filing Date of Aug. 8, 2017, which claims the benefit of priority under 35 USC § 119(e) of U.S. Provisional Patent Application No. 62/371,881 filed on Aug. 8, 2016.

The contents of the above applications are all incorporated by reference as if fully set forth herein in their entirety.

### SEQUENCE LISTING STATEMENT

The contents of the electronic sequence listing (COLT_001_02US_SeqList_ST26.xml: Size: 96,198 bytes; and Date of Creation: Sep. 20, 2024) are herein incorporated by reference in its entirety.

### FIELD AND BACKGROUND OF THE INVENTION

The present invention, in some embodiments thereof, relates to bacterial systems for analyzing ubiquitylated polypeptides.

Ubiquitin (Ub) plays a pivotal role in numerous aspects of cellular processes. Therefore, aberrations in the Ub system are involved in a large number of pathologies, including various forms of cancer such as breast and colon cancer, neurodegenerative diseases such as Parkinson's and Alzheimer's diseases, and infectious diseases such as HIV and Ebola. Consequently, there is a critical need for a detailed understanding of the Ub system. Although there have been significant advances in understanding the ubiquitination process, much less is known about the downstream processes. These include substrate recognition by specific enzymatic interactions in the Ub system, and specific interactions between these enzymes and their substrates. In humans, for example, there are 2 E1 Ub-activating enzymes, 34 Ub-conjugating enzymes and more than 600 E3 Ub-ligases. These enzymes work on presumably several thousands of protein substrates, where specificity is mainly achieved by the E2:E3 and E3:Substrates interactions.

Another factor which impedes the researchers' efforts to fully characterize Ub cascades is the presence of deubiquitinating enzymes (DUBs) which rapidly reverse the ubiquitination signal. The half-life time of ubiquitylated proteins is thus extremely short. Specifically, it has been shown that about 100 DUBs that exist reverse the modification in a highly specific manner.

Background art includes Keren-Kaplan et al., The EMBO Journal (2012) 31, 378-390 and Su et al., J Immunol 2006:177:7559-7566.

### SUMMARY OF THE INVENTION

According to an aspect of some embodiments of the present invention there is provided a method of identifying an agent which regulates the activity or amount of a ubiquitinating enzyme or deubiquitinating enzyme comprising:
(a) contacting a bacterial cell with the agent, wherein the bacterial cell outputs a detectable or selectable signal which correlates with the ubiquitination level of a substrate; and
(b) measuring the level or the rate of accumulation of the detectable or selectable signal, wherein a change in the level as compared to the level in the absence of the agent, is indicative of an agent which regulates the activity or amount of the ubiquitinating or deubiquitinating enzyme.

According to an aspect of some embodiments of the present invention there is provided a method of determining whether an enzyme is capable of ubiquitinating a test substrate, the method comprising
(a) expressing the enzyme in a bacterial cell;
(b) expressing ubiquitin in the bacterial cell, wherein the ubiquitin is attached to a first polypeptide fragment;
(c) expressing the test substrate in the bacterial cell, wherein the substrate is attached to a second polypeptide fragment, wherein the first polypeptide fragment associates with the second polypeptide fragment to generate a reporter polypeptide on ubiquitination of the test substrate; and
(d) analyzing for the presence of the reporter polypeptide in the bacterial cell, wherein a presence of the reporter polypeptide is indicative that the enzyme is capable of ubiquitinating the test substrate.

According to an aspect of some embodiments of the present invention there is provided a method of identifying a polypeptide substrate for a ubiquitinating enzyme, the method comprising:
(a) expressing a plurality of candidate polypeptide substrates in a bacterial cell population, wherein each of the candidate polypeptide substrates is attached to an identical first polypeptide fragment;
(b) expressing the ubiquitinating enzyme in the bacterial cell population;
(c) expressing ubiquitin in the bacterial cell population, wherein the ubiquitin is attached to a second polypeptide fragment, wherein the second polypeptide fragment associates with the first polypeptide fragment to generate a reporter polypeptide on ubiquitination of the substrate; and
(d) analyzing in bacterial colonies of the bacterial cell population for a presence or absence of the reporter polypeptide, wherein a presence of the reporter polypeptide is indicative of expression of a substrate for the ubiquitinating enzyme.

According to an aspect of some embodiments of the present invention there is provided a kit comprising:
(i) a first polynucleotide which encodes a first polypeptide fragment which is operably linked to a bacterial regulatory sequence, and a cloning site, wherein a position of the cloning site is selected such that upon insertion of a sequence which encodes a test polypeptide into the cloning site, following expression in a bacterial cell, a fusion protein is generated which comprises the test polypeptide in frame with the first polypeptide fragment; and
(ii) a second polynucleotide comprising a second nucleic acid sequence encoding a second polypeptide fragment which is attached to ubiquitin, the second nucleic acid sequence being operably linked to a bacterial regulatory sequence, wherein the first polypeptide fragment associates with the second polypeptide fragment to generate a reporter polypeptide dependent on ubiquitination of the test polypeptide.

According to one embodiment, the ubiquitinating enzyme is a ubiquitin E3-ligase,

According to another embodiment, the ubiquitin E3-ligase is selected from the group consisting of Siah2, Smurf1, MDM2, BRCA1, PARKIN, UBE3A, TRIM5, NEDD4, UBR5 and Huwe1.

According to one embodiment, the ubiquitin E3-ligase is selected from the group consisting of Siah2, PARKIN, Smurf1, MDM2, BRCA1, MURFI, TRIM32 ITCH, UBE3B and UBE3A.

According to one embodiment, the substrate is selected from the group consisting of PHD3, SPROUTY2, Mitofusin 1, 2, MIRO, NEMO, SMADs, TβR-I, P53, S5A, HHR23, EPHEXIN5, ARC, PPARα, cyclin-β, Cdc25C, Calmodulin.

According to one embodiment, the regulates comprises downregulates.

According to one embodiment, the regulates comprises upregulates.

According to one embodiment, the ubiquitinating enzyme further comprises an E1 ligase and an E2 ligase.

According to one embodiment, the bacterial cell expresses:
(a) the ubiquitinating enzyme or the deubiquitinating enzyme;
(b) ubiquitin attached to a first polypeptide fragment; and
(c) the substrate attached to a second polypeptide fragment, wherein the first polypeptide fragment associates with the second polypeptide fragment to generate a reporter polypeptide on ubiquitination of the substrate.

According to one embodiment, the reporter polypeptide comprises a selectable polypeptide.

According to one embodiment, the selectable polypeptide is a split antibiotic resistance polypeptide.

According to one embodiment, the antibiotic resistance polypeptide is DHFR or B lactamase.

According to one embodiment, the first polypeptide fragment is attached to the ubiquitin via a linker.

According to one embodiment, the second polypeptide fragment is attached to the substrate via a linker.

According to one embodiment, the reporter polypeptide is an optically detectable signal.

According to one embodiment, the detectable polypeptide is selected from the group consisting of a split fluorescent polypeptide, a split luminescent polypeptide and a split phosphorescent polypeptide.

According to one embodiment, the analyzing is effected by bimolecular complementation of an antibiotic resistance protein.

According to one embodiment, the method further comprises expressing all the enzymes of the ubiquitinating enzyme cascade of the enzyme.

According to one embodiment, the reporter polypeptide is a detectable polypeptide or a selectable polypeptide.

According to one embodiment, the enzyme is selected from the group consisting of E3 ligase, ubiquitin E1-activating enzyme and ubiquitin E2 conjugating enzyme.

According to one embodiment, the test substrate comprises an E3 ligase or Rpn10.

According to one embodiment, the enzyme is a ubiquitin E1-activating enzyme, the method further comprises expressing in the bacterial cell a ubiquitin E2 conjugating enzyme and ubiquitin E3-ligase.

According to one embodiment, the selectable polypeptide is a split antibiotic resistance polypeptide.

According to one embodiment, the antibiotic resistance polypeptide is DHFR or B lactamase.

According to one embodiment, the first polypeptide fragment is attached to the ubiquitin via a linker.

According to one embodiment, the second polypeptide fragment is attached to the substrate via a linker.

According to one embodiment, the detectable polypeptide is an optically detectable signal.

According to one embodiment, the detectable polypeptide is selected from the group consisting of a split fluorescent polypeptide, a split luminescent polypeptide and a split phosphorescent polypeptide.

According to one embodiment, the analyzing is effected by bimolecular complementation of an antibiotic resistance protein.

According to one embodiment, the first and the second polynucleotide comprise a bacterial origin of replication.

According to one embodiment, the reporter polypeptide is a selectable polypeptide.

According to one embodiment, the reporter polypeptide comprises a selection or detectable polypeptide.

According to one embodiment, the kit further comprises a third polynucleotide which encodes at least one ubiquitinating enzyme.

According to one embodiment, the first polynucleotide and/or the second polynucleotide comprises a sequence which encodes at least one ubiquitinating enzyme.

According to one embodiment, the at least one ubiquitinating enzyme comprises ubiquitin E1-activating enzyme or ubiquitin E2-conjugating enzyme.

According to one embodiment, the at least one ubiquitinating enzyme comprises ubiquitin E1-activating enzyme and ubiquitin E2-conjugating enzyme.

According to one embodiment, the at least one ubiquitinating enzyme comprises E3 ligase.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1D—Bacterial genetics approach for selection of ubiquitylated proteins.

(A) A scheme demonstrates the constructed system for selection in bacteria. The ubiquitination apparatus is expressed from two compatible plasmids each harbors different antibiotic resistance and origin of replication, facilitating co-transformation and selection of the vectors regardless of ubiquitination. pND-Ub denotes the N-terminal fragment of DHFR fused to Ub. pCD-Sub denotes the C-terminal fragment of DHFR fused to a ubiquitination substrate. A complete system confers antibiotic resistance and bacterial growth in the presence of trimethoprim (TRIM). Red cross represents bacteria that express an incomplete system and therefore are not resistant to the selective media. (B) Demonstrates the system functionality. Antibiotic resistance (10 μgr/ml TRIM) is attained only when a complete ubiquitination cascade of Vps9 including wheat Uba1, yeast Ubc4, and yeast Rsp5 are co-expressed with split DHRF fragments fused to Ub and Vps9. (C) Shows that cognate E3-ligase (Rsp5) is necessary for Vps9 ubiquitination and antibiotic resistance. (D) Non-covalent interaction between Vps9 and Ub does not confer resistance for TRIM concentrations above 0.1 μg/ml. Ubiquitination—refers to complete ubiquitination cascade; binding—refers to incomplete ubiquitination cascade (ΔΕ1, ΔΕ2).

FIGS. 2A-2G—Identification and characterization of NleG6-3 as E3-ligase.

(A) Bacteria that co-express cDHFR fusion to the E3-ligases Rsp5 (representative of the HECT family) with its cognate ubiquitination apparatus including Ubc4 is labeled ‘complete’. Deletions of E1, E2 or Ub are indicated. (B) Bacteria that co-express cDHFR fusion to the E3-ligases Siah2 (representative of the RING family) with its cognate ubiquitination apparatus including UbcH5a is labeled ‘complete’. Deletions of E1, E2, Ub or substitution of the UbcH5a with Cdc34 are indicated. The right panel shows Siah2 inhibition by menadione. (C) Shows sequence alignment derived from PSI-BLAST search using human CHIP as probe against the EHEC proteome. ECs3488 CSLYDKDTLVQLVETGGAHPLSREPITESMIMR—SEQ ID NO: 1; ECs2156CTLFDAAAFSRLVGEGLPHPLTREPITASIIVK—SEQ ID NO: 2; HS_CHIP ITYDRKDIEEHLQRVGHFDPVTRSPLTQEQLIP—SEQ ID NO: 3. The full length sequence of the NleG6-3 ligase is as set forth by SEQ ID NO: 4.

(D) Yeast E2s scan for the E3-ligase ECs3488 (NleG6-3). (E) Self-ubiquitination of NleG6-3 by human UbcH5b/c. (F) Shows homology based model of NleG6-3 (orang) super imposed on the structure of EHEC NleG2-3 (blue) and Human CHIP:Ubch5b complex grey and magenta (respectively). Zooming into the predicted interface with highlighted critical binding residues. (G) Employment of the selection system for mutational analysis of the predicted E2:E3 interface.

FIGS. 3A-3I—The bacterial selection system facilitates the identification and characterization of Ub-receptors. UBDs promote self-ubiquitination and therefore can be detected by the bacterial selection system. The CDHER was fused to various UBDs in the pCD-Sub, and co-expressed in the selection system without E3-ligase. (A) Ubiquitination promoted by Hrs was used to validate the system performance. (B) Structure of the Hrs dsUIM:Ub complex (PDB code 2D3G) shows the interacting residues. (C) Western blot analysis demonstrates the formation of a covalent bond between Ub and GST-dsUIM fusion. His6-Ub was co-expressed with E1 and Ubc4 along with GST-dsUIM. The protein was purified on GSH beads and subjected for western blot with anti GST (red) and anti-Histag (green). The yellow band that migrated at the molecular size of the ubiquity lated protein was detected by both antibodies. (D) The E1 inhibitor PYR41 attenuates bacterial growth only in the non-permissive conditions. Bacteria were grown in 96-well plates supplemented with PYR41 and/or TRIM as indicated. The relative growth rates at the log phase are shown with standard deviation (n=9). (E-H) Show the growth phenotypes of assorted UBDs in the selection system. e. Yeast Rpn10; f. Human STAM1-UIM; g. Human Alix-V domain; h. Yeast Hsel-VHS domain. (I) The binding of Ub to the VHS domains of human STAM1, GGAL and GGA2 were examined in the selection system. Critical STAM1-VHS residues known to bind Ub were mutated and the growth phenotype was tested.

FIGS. 4A-4J—Structural insight into a predicted ENTH:Ub interface.

(A-B) The selection system identified Epsinl-ENTH domains from yeast (Saccharomyces cerevisiae) Entl and from zebrafish (Danio rerio) Epn1 function as UBDs. Co-expression of these ENTH domains with the ubiquitination apparatus in the selection system conferred antibiotic resistance and promoted bacterial growth under non-permissive conditions. Structures of the zebrafish and yeast Epsin1 ENTH domains are presented. (C-D) Show the 2mFo-DFc sigma-A maps of the fish and the yeast proteins to 2.2 and 1.8 σ (respectively) of the final refined models. (E) The superimposition of the fish and the yeast ENTH structures on top of the STAM1-VHS:Ub complex (pdb code 3LDZ). Average Cα RMSD are 1.5 Å and 1.7 Å, respectively. The models were energetically minimized and carefully inspected. (F) Zoom in into crystal structure of the VHS:Ub interface (3LDZ). (G-H) Model of the ENTH:Ub complexes. (I) and (J) show zoom in into the interface of the model complexes.

FIGS. 5A-5I—Characterization of the ENTH:Ub binding interface.

Structure-based mutants at the predicted ENTH:Ub interface were constructed and characterized. (A) Growth phenotypes of the zebrafish ENTH and Ub mutants in the selection system. (B) Growth curves of Ub wild type and mutants derived from the time-lapse scanning of the spots (density was analyzed by Fiji). (C) As shown in b, but for ENTH mutants. (D) As shown in b and c but for reciprocal mutations. (E) Growth phenotypes of the yeast ENTH and Ub mutants in the selection system. (F) As shown in b, but for yeast ENTH and Ub mutants. (G) The ubiquitination yield for wild-type and indicated mutants of the yeast His6-MBP-ENTH were evaluated. The apo and ubiquity lated proteins were purified on amylose beads as described¹⁷, resolved on SDS-PAGE and detected by western blot with anti His-tag antibody. (H) Quantification of the ubiquitination yields shown in (G). The ubiquitylated apo ratio is presented as a percentage of the wild-type ratio. Values were averaged from four independent experiments, and standard deviation error bars are presented. (I) Surface Plasmon Resonance (SPR) analysis of the yeast ENTH:Ub binding affinity of wild-type and mutant proteins. Fitting to binding curves was carried out with a single-site-binding model using the OriginLab software. Standard errors derived from three independent measurements are indicated.

FIGS. 6A-6D—Sem1 is a ubiquitination substrate of Rsp5.

Screening of pCD-Sub yeast fusion library revealed Sem1 as potential ubiquitination substrate of Rsp5 in the bacterial selection system. (A) Spots growth at the indicated hours post seeding shows that Rsp5 significantly promotes Sem1 ubiquitination (compare complete system that contains Rsp5 with a ΔRsp5 cascades). A 30 minutes intervals time-lapse movie of the scans can be found in the supplementary data. (B) Growth curves derived from quantification of the scans using ‘Time Series Analyzer’in Fiji. Values are average of eight spots with standard deviation bars. (C) Detection of Sem1 ubiquitination in E. coli. Purified His₆-MBP-Sem1 from E. coli that co-express ubiquitination apparatus ¹⁷ was resolved on SDS-PAGE and detected by western-bolt with anti-Histag antibody. (D) Sem1 is a ubiquitination substrate of Rsp5 in vivo in yeast. His₆-Sem1 was expressed from Gal inducible promoter in wild-type or rsp5-1 yeast cells. Cultures grew at 25° C. (permissive conditions). Prior induction temperature was shift to 37° C. or remained as indicated. His₆-Sem1 was purified under denatured conditions, resolved and detected as in (C).

FIGS. 7A-7F—Functional analysis of the vWA:Ub binding patch. (A) Scheme showing a bacterial genetic selection system for ubiquitination (B) Ubiquitination addicted bacterial growth on selective (+Trimethoprim) or non-selective plates. A single scan of the plates 98 hours post seeding is shown. (C) Shows quantitation of ubiquitination dependent bacterial growth. Average density of individual spots monitored by scanning the plates in 1 hour intervals was plotted. Efficiency was calculated as the max growth density divided by the time of half max growth. NSG—no significant growth. (D) Shows a representative SPR response curves for the vWA:Ub complex. (E) Single model binding analysis of SPR affinity measurements of Rpn10:Ub wt and the indicated mutants. Kd values are indicated (right; NB—no binding). (F) Comparison between the relative growth of wt or mutant spots and the SPR measured association constants (Kₐ). Pearson product-moment correlation coefficient is r=0.99 (p<0.001).

FIGS. 8A-8B—Schemes show two of the major hurdles that pose challenges in assigning specific associations of components along ubiquitin cascades.

(A) Ubiquitination is rapidly reversed by deubiquitylases. In human there are about hundred deubiquitinating enzymes (DUBs) which efficiently and rapidly remove the ubiquitin moiety from targeted proteins.

(B) Ubiquitination cascades are multiplex. For example eight different substrates were found for the BRCA1 E3-ligase. Similarly, 7 different E3-ligases were demonstrated to ubiquitylate p53.

FIG. 9—Architecture and sequence of the linkers connecting the DHFR fragments to Ub and substrates. Schematic illustration of the constructs used in the selection system. pND-Ub vectors contain a N-Terminal DHFR fragment fused through the flexible linker (linker 1) to Ub. In the pCD-Sub the C-Terminal fragment of the DHFR was fused through the flexible linker (linker 2) to the His₆-MBP-substrate or directly to the substrate.

Linker 1:
SEQ ID NO: 5
LIKAAQRAREAERDLAAAVAQAAAGQAVPRAAPRQ-
Linker 2:
SEQ ID NO: 6
GGSAGSGSGAGSGSGGSAGSSGSSGASSG-.

FIG. 10—Domain architecture of membrane associated Ub-receptors. Ub moieties mark the binding sites (orange circled U's).

FIGS. 11A-11E—Ent1-ENTH domain directly binds ubiquitin. (A) Shows crosslinking assay of Ent1-ENTH domain with increased concentrations of Ub. A mild crosslinker, disuccinimidyl suberate (DSS) was used. Reactions were resolved by SDS-PAGE and Coomassie blue stained. (B) Shows crosslinking assay like in (A) of Ent1-ENTH domain with wild-type and Ub 144E mutant for various incubation times (as indicated). (C) Scheme of yeast and zebrafish Epsin-1 derivative constructs. (D). Ubiquitination of yeast Ent1 derivatives. (E). Ubiquitination of zebrafish Epn1 derivatives. His₆-Ub was co-expressed with E1 and Ubc4 along with GST-Epsin1 derivatives. Proteins were purified on GSH-beads and ubiquitination was detected by western blot using anti-Histag antibody as described in ¹⁷.

FIGS. 12A-12B—Structural divergence within the ENTH domains. The coordinates of several ENTH domains including yeast (magenta), zebrafish (white), and three structures of rat ENTH domains (cyan, red and yellow), were superposed. The axis of helix-8 from each of the structures were calculated. Then the angles among the derived helices were compared (A). Structural differences between the loops tethering helices number 3 and 4 in yeast and zebrafish ENTH domains are presented (B).

FIG. 13—ENTH/VHS domains can simultaneously associate with membranes and ubiquitin. Superimposing the ENTH complex with the lipid phosphatidylinositol-4,5-bisphosphate, GGA3-VHS complex with the Mannose-6P Receptor tail and the STAM1-VHS:Ub shows that membrane and Ub associations occur at opposite sides of the domain and therefore can occur simultaneously.

FIGS. 14A-14C—Surface Plasmon Resonance (raw data). Sonograms of the SPR responses of the WT and the indicated mutants are presented.

FIGS. 15A-15D—a schematic view of the split CAT bacterial selection system for ubiquitination.

A. The reaction executed by CAT (i.e. transfer of acetyl group from acetyl-CoA to chloramphenicol is shown.

B. Structure representation of assembled split CAT with chloramphenicol.

C. Linear representation of the split-CAT system.

D. cartoon view of the split CAT bacterial selection system.

FIGS. 16A-16D—Split-CAT system detects ubiquitination.

A. Shows E3 independent uniquitination of the Ub-binding domain VHS of yeast Hse1.

B. The split CAT system shows significant higher growth efficiency that shortens the experimental time. Shown is UBE3A dependent ubiquitination of Rpn10 in the split CAT and DHFR selection system.

C. Mutation in UBE3A E3 ligase (G738E) that causes Angelman-syndrome phenotype.

D. The split CAT system facilitates the study of E3 ligases that cannot be purified from E. Coli such as UBE3B. Shown is UBE3B dependent ubiquitination of Rpn10 in the split CAT system. Kaufman syndrome mutation (G781R) in UBE3B shows a phenotype.

FIGS. 17A-17B illustrate the DNA sequence and the translation products of CAT_{I}(FIG. 17A—SEQ ID NO: 63 and SEQ ID NO: 64) and the split-CAT_{I} fragments (FIG. 17B—SEQ ID NOs: 65-68). Arrow marks the cleavage site that was chosen for the split protein fragments (top). The stop codon after residue Q30 and the initiation codon prior residue C31 are shown in the split protein fragments (bottom).

### DESCRIPTION OF SPECIFIC EMBODIMENTS OF THE INVENTION

The present invention, in some embodiments thereof, relates to bacterial systems for analyzing the ubiquitination of polypeptides.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

About one-third of the eukaryotic proteome undergoes ubiquitination, but most of the enzymatic cascades leading to substrate modification are still unknown. The present inventors have invented a genetic selection tool that utilizes E. coli, which lack deubiquity lases, to identify interactions along ubiquitination cascades. Co-expression of split antibiotic resistance protein tethered to ubiquitin and ubiquitination target together with a functional ubiquitination apparatus results in a covalent assembly of the resistance protein, giving rise to bacterial growth on selective media. The capability of the screening tool for small molecule modulators, in a high-throughput format is demonstrated herein. In addition, using a complete E2-library in the selection system, the present inventors identified and characterized an E3-ligase from the pathogenic bacteria EHEC. Furthermore, the present inventors identified ENTH as an ultraweak ubiquitin-binding domain, demonstrating the tool's high sensitivity. X-ray crystallography combined with bacterial selection studies facilitates structure-function analysis of the ENTH:ubiquitin interface. Moreover, study of the Rpn10:Ub non-covalent interaction (FIGS. 7A-7F) demonstrated an excellent correlation between the growth efficiency under restrictive conditions of wild-type and mutants in the genetic selection system and the binding affinities measured by Surface Plasmon Resonance (SPR). FIG. 7F shows a comparison between the relative growth efficiency on selective media and the relative SPR association constants (Kₐ). Linear regression provides a Pearson correlation coefficient of r=0.99 (p<0.001).

These finding demonstrate that one can employ the system for drug discovery and improvements (hit-to-lead) as the ubiquitination dependent growth is highly correlated with the affinities along the cascade. Whilst further reducing the present invention to practice, the present inventors constructed and screened a yeast fusion library and discovered a novel physiological ubiquitination. Collectively, the developed system provides a robust high-throughput approach for genetic studies of ubiquitination cascades.

Thus, according to one aspect of the present invention there is provided a method of identifying an agent which regulates the activity or amount of a ubiquitinating enzyme or deubiquitinating enzyme comprising:
(a) contacting a bacterial cell with the agent, wherein the bacterial cell outputs a detectable or selectable signal which correlates with the ubiquitination level of a substrate; and
(b) measuring the level or the rate of accumulation of the detectable or selectable signal, wherein a change in the level as compared to the level in the absence of the agent, is indicative of an agent which regulates the activity or amount of the ubiquitinating or deubiquitinating enzyme.

The method of this aspect of the present invention can screen for agents that upregulate or downregulate the activity and/or amount of the enzyme.

Agents that may be screened include small molecule agents, peptide agents, nucleic acid agents, antibodies, proteins, chemotherapeutic agents etc.

The screening assay of this aspect of the present invention uses bacteria that have been genetically modified to output a detectable or selectable signal which correlates with the ubiquitination level of a substrate.

Ubiquitination takes place by a cascade of enzyme activity (i.e. a plurality of enzymes which work together to bring about the same function—ubiquitination). For example, E1 activates the Ub; then Ub is transferred to E2. E2 together with E3 (or in many cases transfer the Ub to E3) recognize a specific target and ligate the Ub to the target protein.

Below is a list of the components of the assay which are expressed by the genetically modified bacteria of this aspect of the present invention, each of which will be described in detail herein below.
1. Ubiquitin;
2. Detectable signal;
3. At least one ubiquitinating or deubiquitinating enzyme; and
4. Substrate (target for ubiquitylation).

Any bacteria can be used for this assay so long as it lacks endogenous deubiquitinase activity and preferably also endogenous ubitquitinase activity. In one embodiment, the bacteria has at least 10 fold less endogenous deubiquitinase activity and endogenous ubiquitinase activity than a human cell. In another embodiment, the bacteria has at least 20 fold less endogenous deubiquitinase activity and endogenous ubiquitinase activity than a human cell.

Preferably the bacteria lack resistance to the selection markers in the current system. Examples of such bacteria include, but are not limited to E. coli K-12 derivatives including W3110, MG1655, DH5α, JM101, JM19, BL21, B834, XL1-Blue; also other non E. coli bacteria maybe used.

According to a particular embodiment, the bacteria used in the system are of the genus Escherichia, such as for example E. Coli.

In order to express the components of the assay, a polynucleotide sequence encoding the elements described above is preferably ligated into a nucleic acid construct suitable for bacterial cell expression. Such a nucleic acid construct includes a promoter sequence for directing transcription of the polynucleotide sequence in the cell in a constitutive or inducible manner.

The phrase “an isolated polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase; or synthetically synthesized by assembled from short oligonucleotide.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, a typical cloning vector may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

Exemplary promoters contemplated by the present invention include, but are not limited to polyoma, Simian Virus 40 (SV40), adenovirus, retroviruses, hepatitis-B virus and cytomegalovirus promoters. According to a particular embodiment, the promoter is a bacterial promoter.

Constitutive promoters suitable for use with the present invention are promoter sequences which are active under most environmental conditions and most types of bacterial cells such as an unregulated bacteriophage lambda left promoter (pL) or pTac which presents high leakiness.

Enhancer elements can stimulate transcription up to 1,000 fold from linked homologous or heterologous promoters. Enhancers are active when placed downstream or upstream from the transcription initiation site. Many enhancing elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

In a preferred embodiment, the vector comprises a bacterial replication of origin.

The expression vector of some embodiments of the invention can further include additional polynucleotide sequences that allow; for example, the translation of several proteins from a single mRNA such as an internal ribosome entry site (IRES) and sequences for genomic integration of the promoter-chimeric polypeptide.

The use of bacterial operon architecture for multi-gene expression, where a single promoter is followed by several open reading frames (ORFs) each contains a ribosome-binding site (Shine-Dalgarno sequence) facilitates the co-expression of the multi-protein complex of the ubiquitination apparatus.

It will be appreciated that the individual elements comprised in the expression vector can be arranged in a variety of configurations. For example, enhancer elements, promoters and the like, and even the polynucleotide sequence(s) encoding the fusion protein can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Exemplary methods of introducing the polynucleotides of the present invention into prokaryotic cells are well known in the art—these include, but are not limited to, transforming with a recombinant bacteriophage DNA, plasmid DNA or cosmid DNA expression vector containing the relevant gene sequences.

Other than containing the necessary elements for the transcription and translation of the inserted coding sequence, the expression construct of some embodiments of the invention can also include sequences engineered to enhance stability, production or isolation of the expressed peptide fragments.

Examples of bacterial constructs include the pET series of E. Coli expression vectors (see for example Studier et al (1990) Methods in Enzymol 185:60-89) in which their T7 promoter was replaced with the constitutive active bacteriophage pH (left promoter). Other vectors that may be used are those that belong to the pZE vector family (e.g. pZE21), those that belong to the pCloDF (containing a pCloDF13 origin) and pACYC (containing a p15A origin of replication).

### Ubiquitin

The term “ubiquitin” as used herein refers to either mammalian ubiquitin having a sequence as set forth in SEQ ID NO: 7 or yeast ubiquitin having a sequence as set forth in SEQ ID NO: 8.

### Detectable Signal

In one example, the detectable signal is a fluorescent protein or an enzyme producing a colorimetric reaction. Exemplary proteins that generate a detectable signal include, but are not limited to green fluorescent protein (Genbank Accession No. AAL33912), alkaline phosphatase (Genbank Accession No. AAK73766), peroxidase (Genbank Accession No. NP_568674), histidine tag (Genbank Accession No. AAK09208), Myc tag (Genbank Accession No. AF329457), biotin ligase tag (Genbank Accession No. NP_561589), orange fluorescent protein (Genbank Accession No. AAL33917), beta galactosidase (Genbank Accession No. NM_125776), Fluorescein isothiocyanate (Genbank Accession No. AAF22695) and strepavidin (Genbank Accession No. S11540).

In another example, the detectable signal is a luminescent protein such as products of bacterial luciferase genes, e.g., the luciferase genes encoded by Vibrio harveyi, Vibrio fischeri, and Xenorhabdus luminescens, the firefly luciferase gene FFlux, and the like.

In one embodiment, the selection is dominant selection, which typically uses a drug to arrest growth of a host cell. Those cells which would express a protein conveying drug resistance would survive the selection. The use of split marker allows the detection of ubiquitination events as further described below.

In order to output a detectable or selectable signal which correlates with the ubiquitination level of a substrate, the present inventors contemplate using a split polypeptide, wherein one fragment of the polypeptide is linked to ubiquitin and the other fragment of the polypeptide is linked to the substrate. When the polypeptide is expressed as two “split” fragments, there is no detectable or selectable signal. However, when the two fragments are brought close enough together (i.e. on ubiquitination of the substrate) they form a functional protein that emits a detectable or selectable signal—i.e. generate a reporter polypeptide.

According to a particular embodiment, the split polypeptide combines to generate a reporter polypeptide which is fluorescent, luminescent, phosphorescent or one that confers antibiotic resistance.

Examples of split polypeptides contemplated by the present invention include, but are not limited to beta lactamase, dihydrofolate reductase (DHFR), focal adhesion kinase, enhanced GFP, horseradish peroxidase, Infrared fluorescent protein IFP1.4 (an engineered chromophore-binding domain (CBD) of a bacteriophytochrome from Deinococcus radiodurans) LacZ (beta-galactosidase) Luciferase, TEV (Tobacco etch virus protease).

According to a particular embodiment the split polypeptide provides resistance to an antibiotic when combined, but the bacteria is susceptible to the antibiotic when split. Preferably, the split polypeptide provides resistance to a bacteristatic antibiotic when combined. Examples of bacteriostatic antibiotics include but are not limited to trimethoprim and chloramphenicol.

In the case of trimethoprim, a split DHFR protein may be expressed. Specifically the use of selective media which lack thymidine, glycine, serine or methionine and contains the trimethoprim antibiotic allows the selection of genes required for the ubiquitination process.

In the case of chloramphenicol, a split chloramphenicol acetyl transferase (CAT) enzyme can be expressed.

As used herein, the term CAT refers to an enzyme (EC 2.3.1.28) that catalyzes the acetyl-S-CoA-dependent acetylation of chloramphenicol at the 3-hydroxyl group.

The CAT of this embodiment may have an amino acid sequence that is at least 90%, 91%, 92%, 93%, 94%, 95%, 96%, 97%, 98%, 99% or 100% homologous or identical to the sequence as set forth in SEQ ID NO: 64, as determined using the Standard protein-protein BLAST [blastp] software of the NCBI. An exemplary DNA sequence that encodes full length CAT is set forth in SEQ ID NO: 63.

In one embodiment the N-terminal fragment comprises a first portion of the catalytic active site of the CAT—e.g. the N terminal fragment typically contains the first 28 or 30 amino acids of the native CAT. The C-terminal fragment comprises the second portion of the catalytic active site of the CAT—for example, the C terminal fragment typically contains the rest of the sequence of the native CAT. The N-terminal fragment associates with the C-terminal fragment to generate an active CAT that is capable of acetylating chloramphenicol.

In one embodiment, the N terminus of the N-terminal fragment is linked to the C terminus of the substrate or ubiquitin (preferably via a linker).

In another embodiment, the C terminus of the C-terminal fragment is linked to the N terminus of the substrate or ubiquitin (preferably via a linker)—see for example FIG. 15C.

Preferably, the first amino acid of the C-terminal fragment is a small amino acid residue—for example cysteine or alanine. Thus, the C terminal fragment may begin with cysteine 31 (wherein the numbering is according to SEQ ID NO: 64), or alanine 29 (wherein the numbering is according to SEQ ID NO: 64). Other small amino acid residues include glycine, alanine, serine, proline, threonine, aspartate and asparagine.

By being small, the first amino acid of the C-terminal fragment after the formyl-methionine is causes the latter to be posttranslationally removed from the N-terminus (of the C-terminus fragment) hence salvaging the active site arrangement as seen by the activity.

In one embodiment, the N-terminal fragment comprises/consists of the amino acid sequence as set forth in SEQ ID NO: 67.

The C-terminal fragment comprises/consists of the amino acid sequence as set forth in SEQ ID NOs: 68.

The N-terminal fragment may be encoded by the nucleic acid sequence as set forth in SEQ ID NO: 65.

The C-terminal fragment may be encoded by the nucleic acid sequence as set forth in SEQ ID NO: 66.

The DNA and protein sequence of an exemplary split CAT is illustrated in FIG. 17B.

As mentioned, in order to generate the recombinant bacteria of the above described embodiment, the bacteria are genetically modified to express at least two polypeptide fragments—one of the polypeptide fragments being the split polypeptide fragment linked to ubiquitin and the other polypeptide fragment being the conjugate pair of the split polypeptide which is linked to the substrate.

In one embodiment, the split polypeptide fragment is directly linked to ubiquitin or substrate. In another embodiment, the split polypeptide fragment is linked to the ubiquitin or substrate via a peptide linker. The linker should be of a length and flexibility that allows functional stability of the reporter polypeptide. The linker is preferably between 10-500 amino acids, more preferably between 20 and 200 amino acids and more preferably between 20-100 amino acids. Exemplary peptide linkers that can be used are set forth in SEQ ID NOs: 5 or 6.

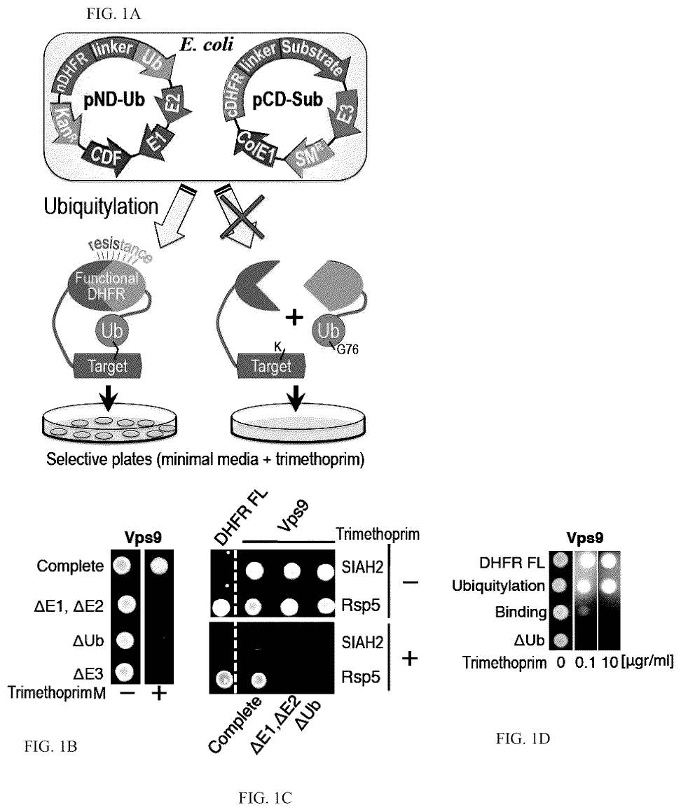

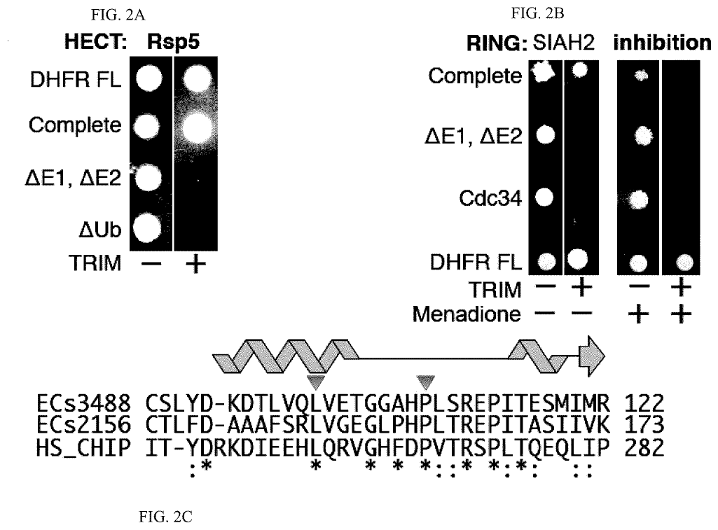

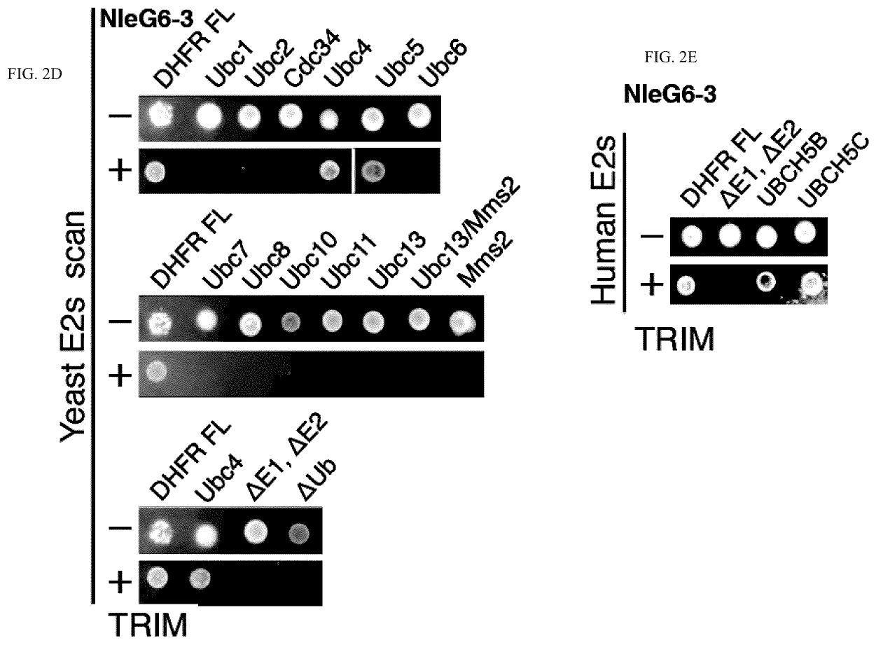

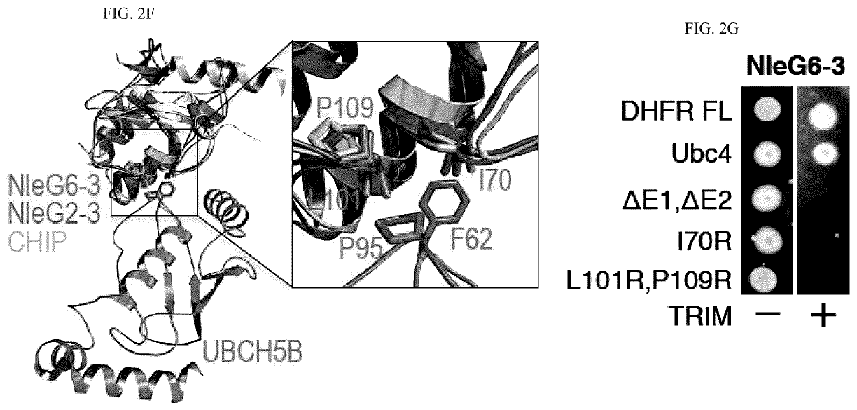

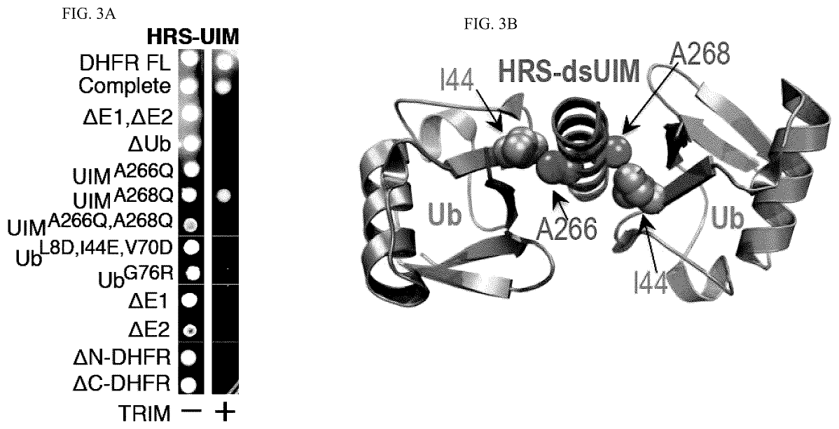

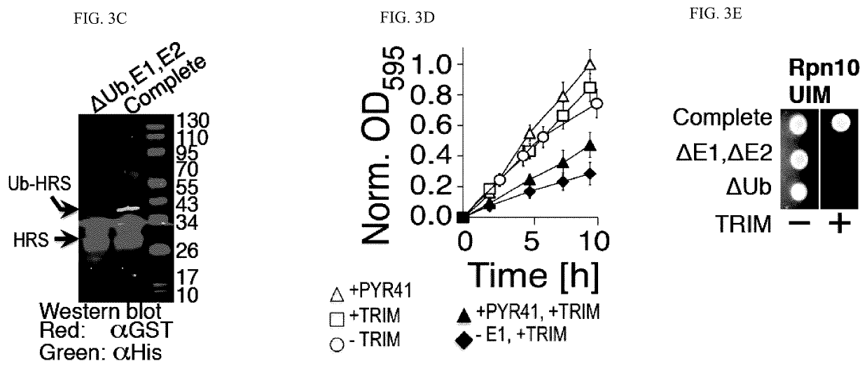

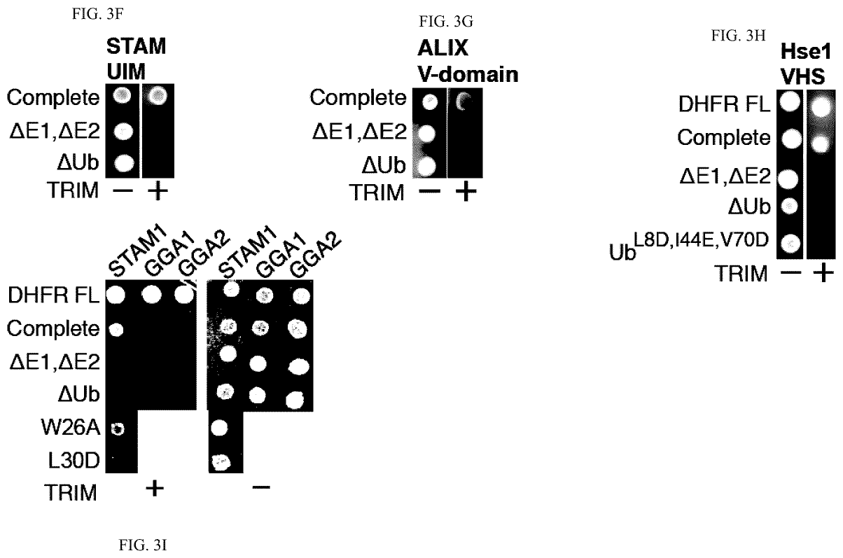

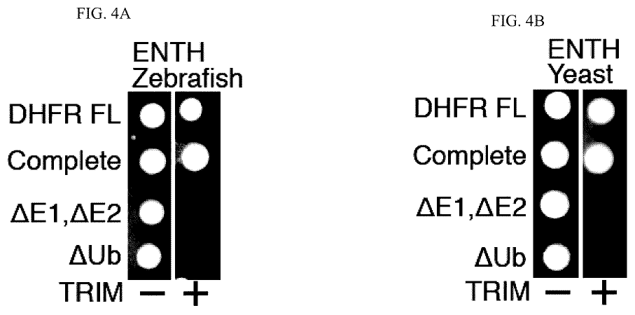

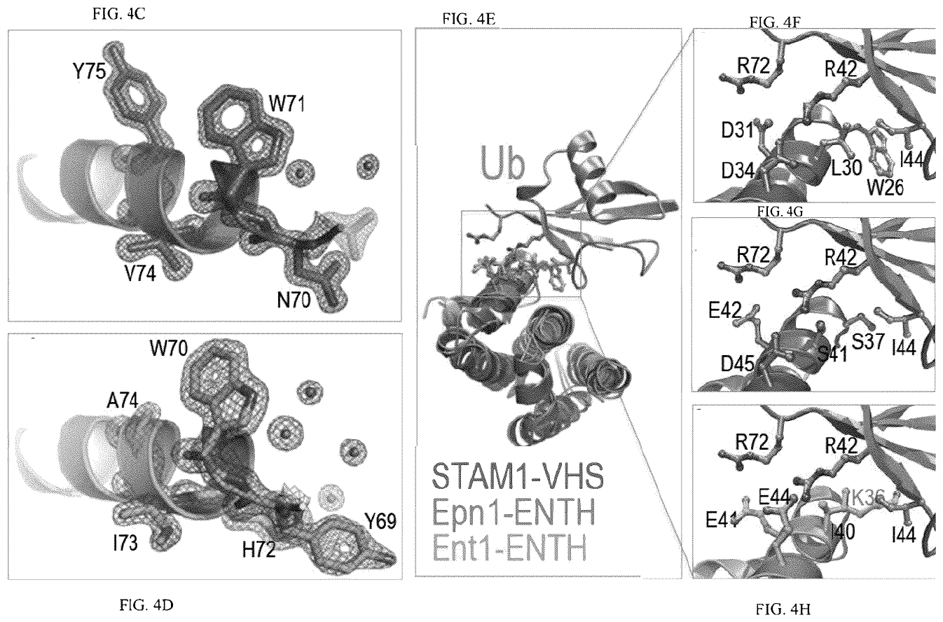

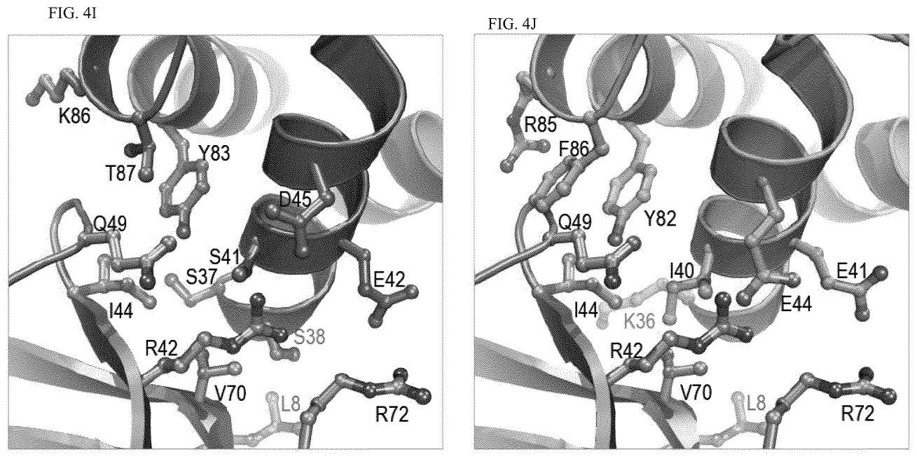

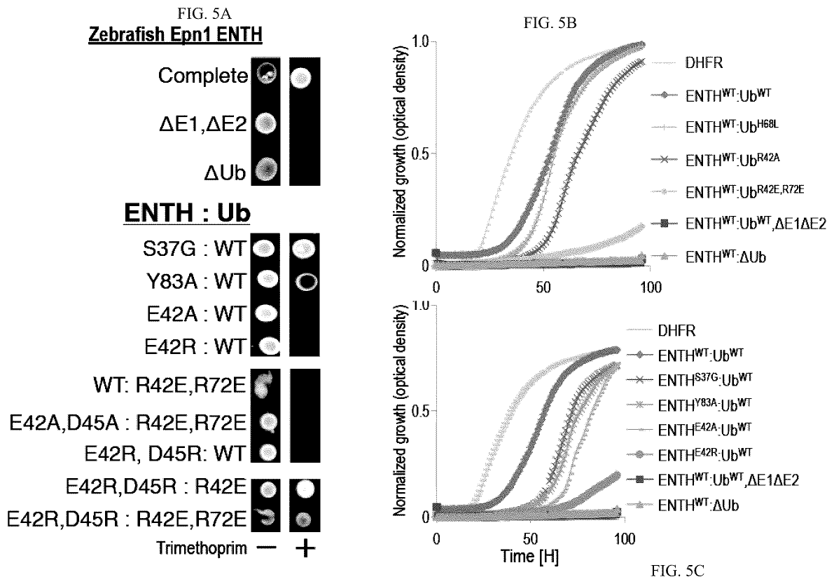

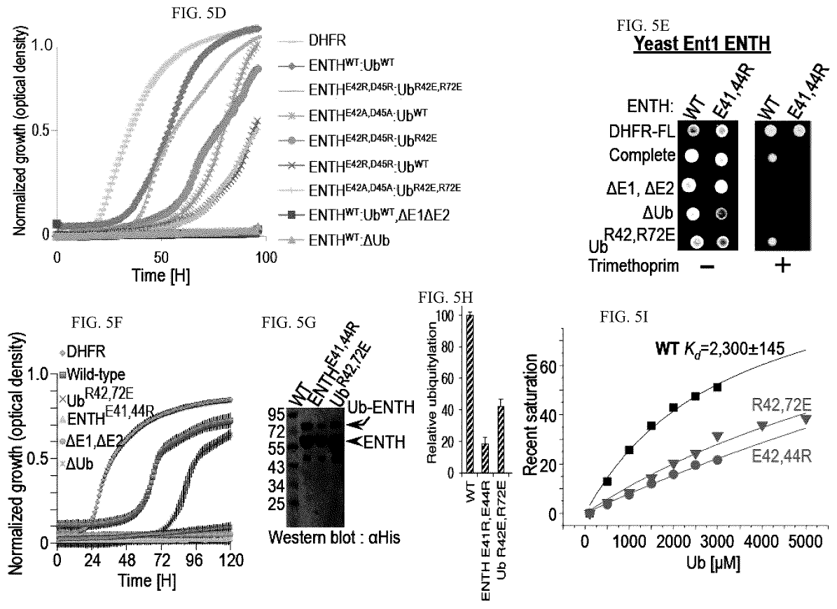

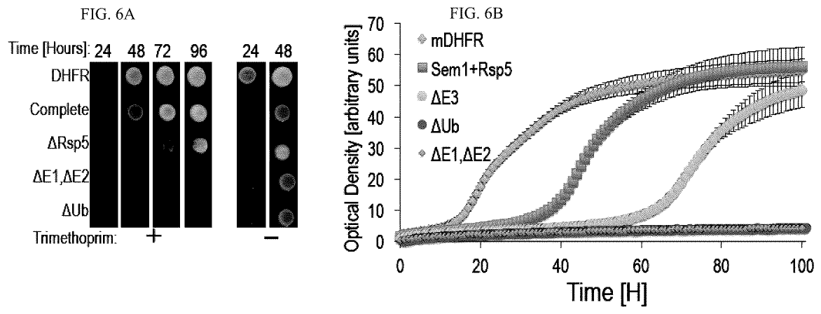

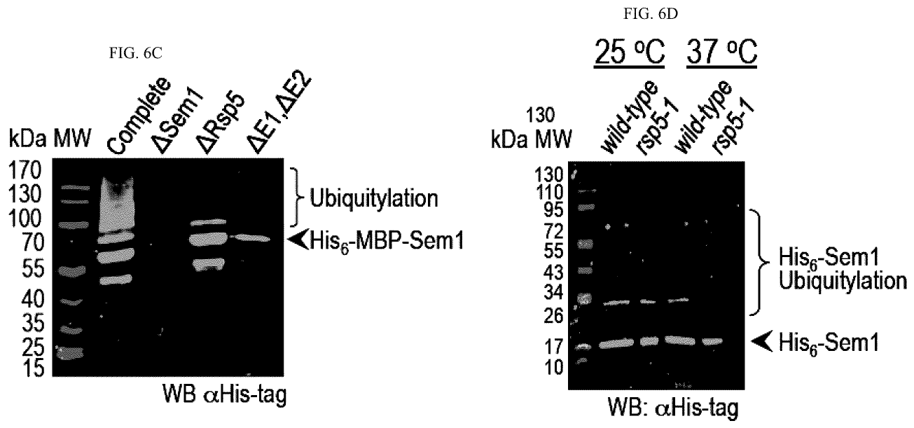

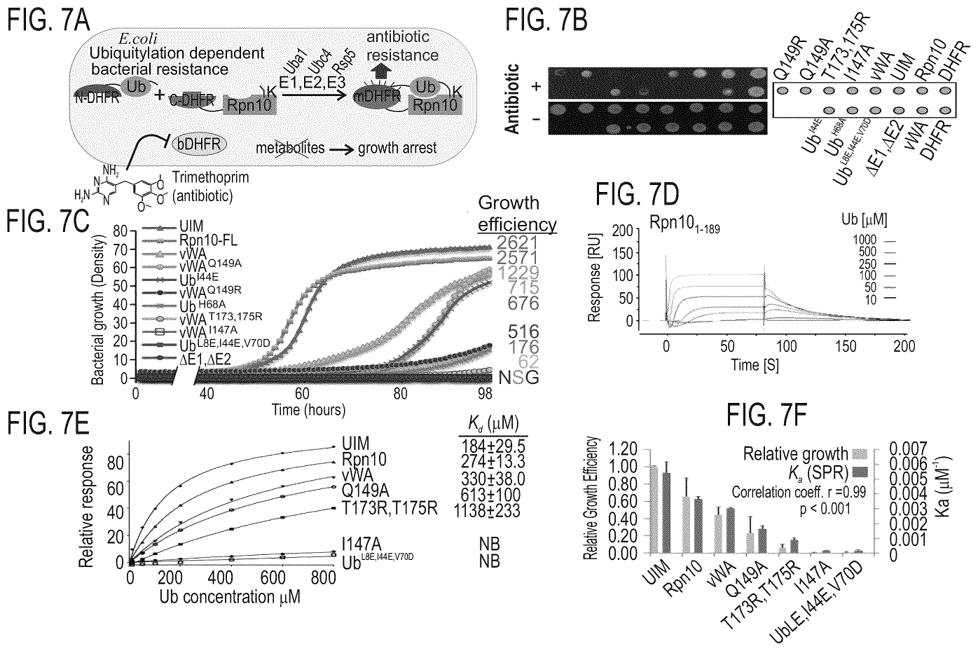

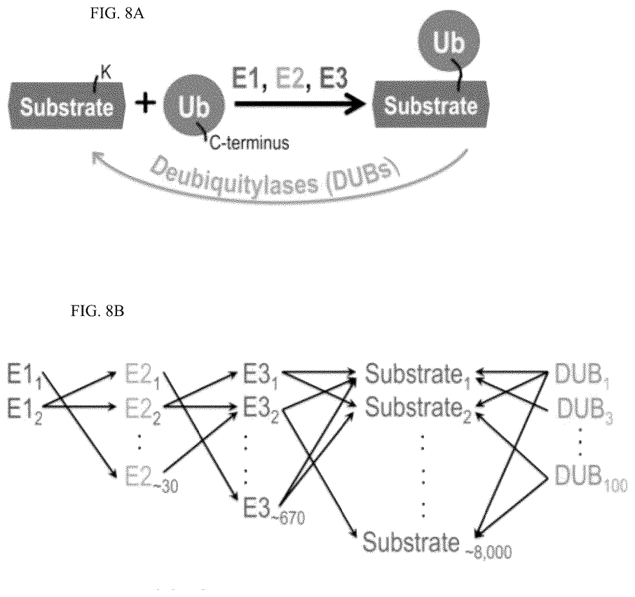

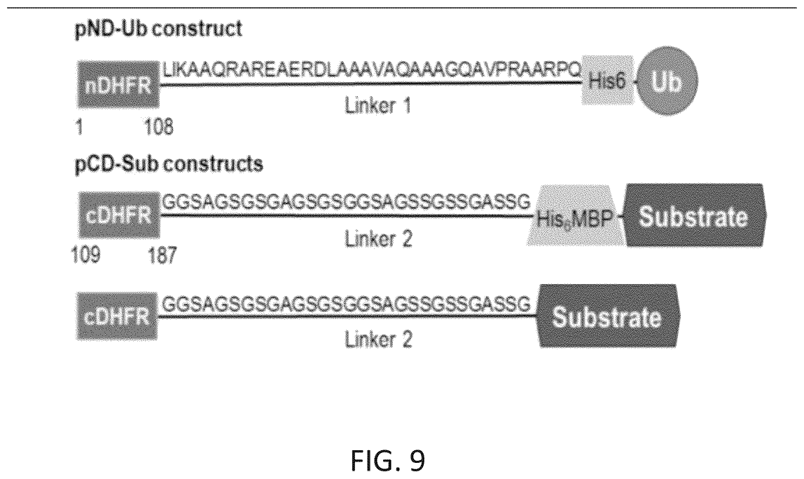

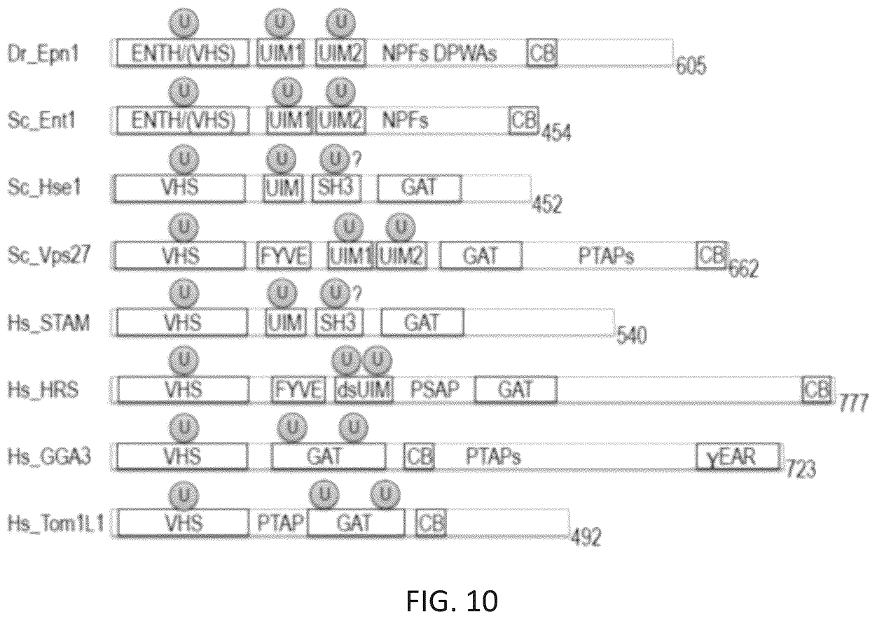

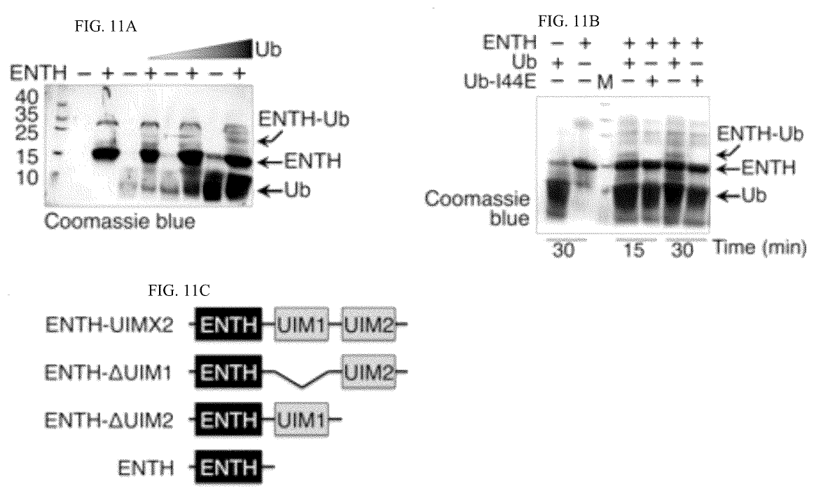

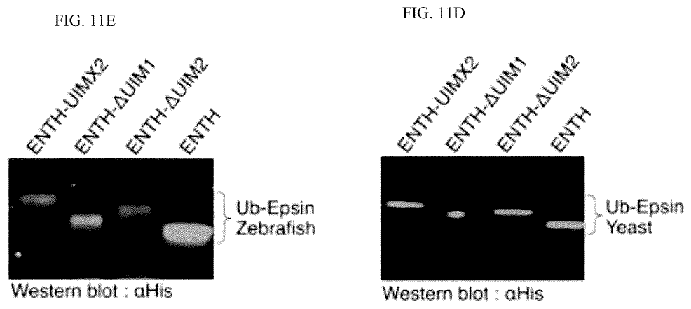

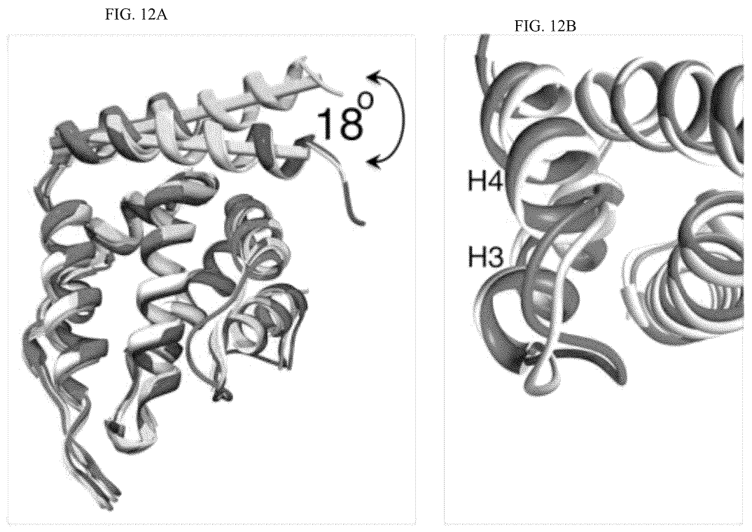

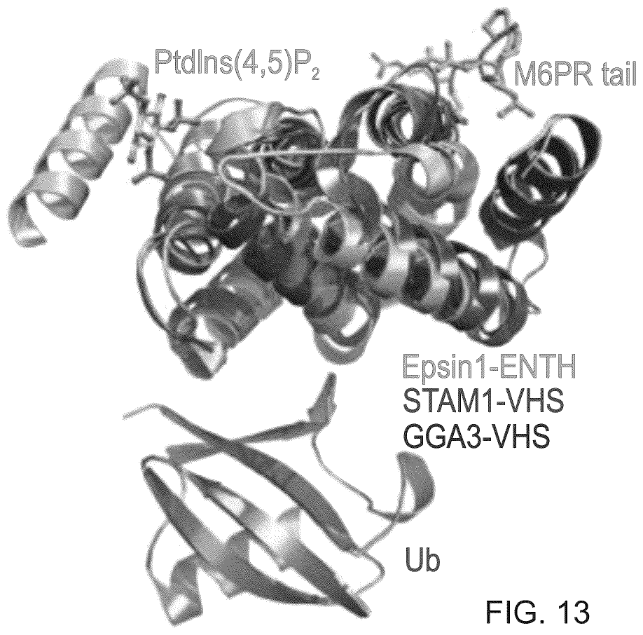

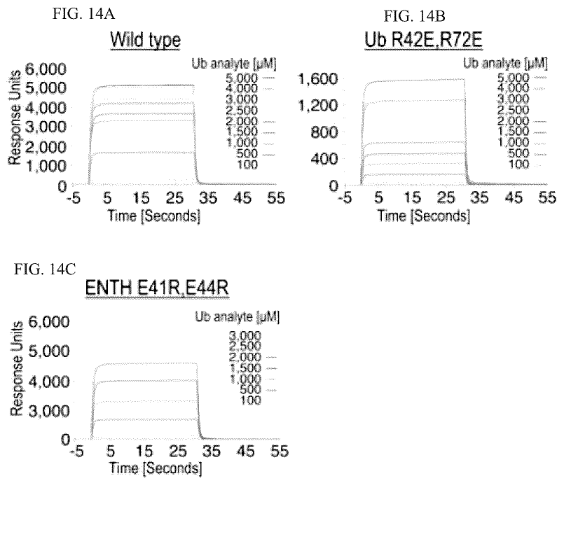

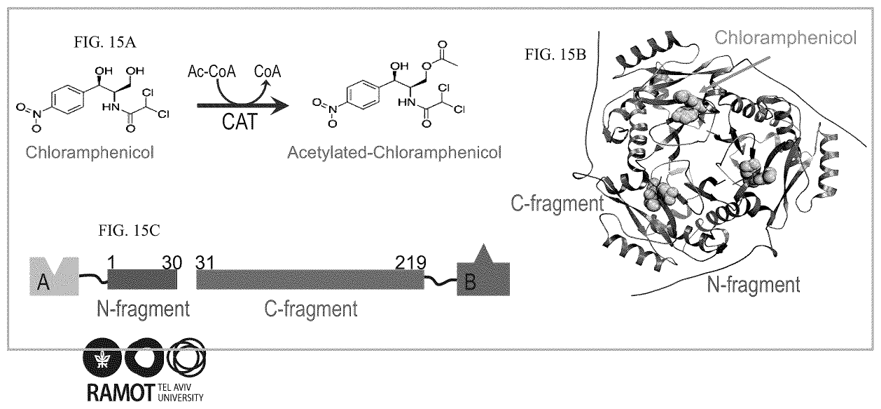

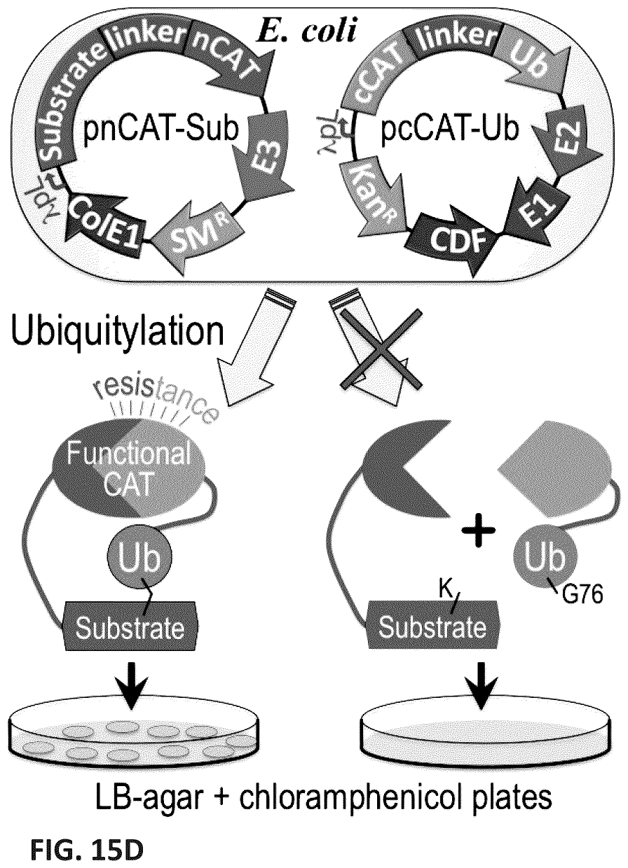

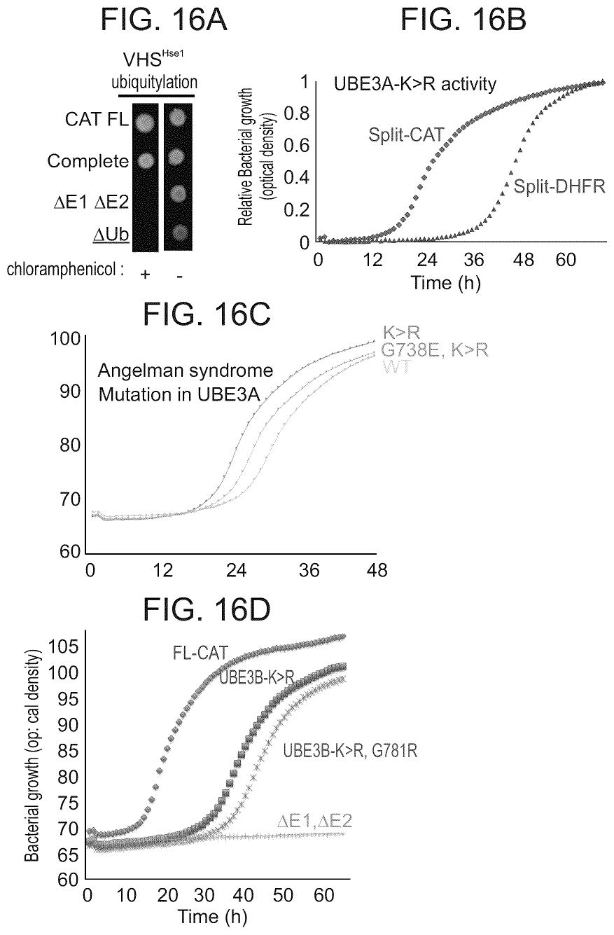

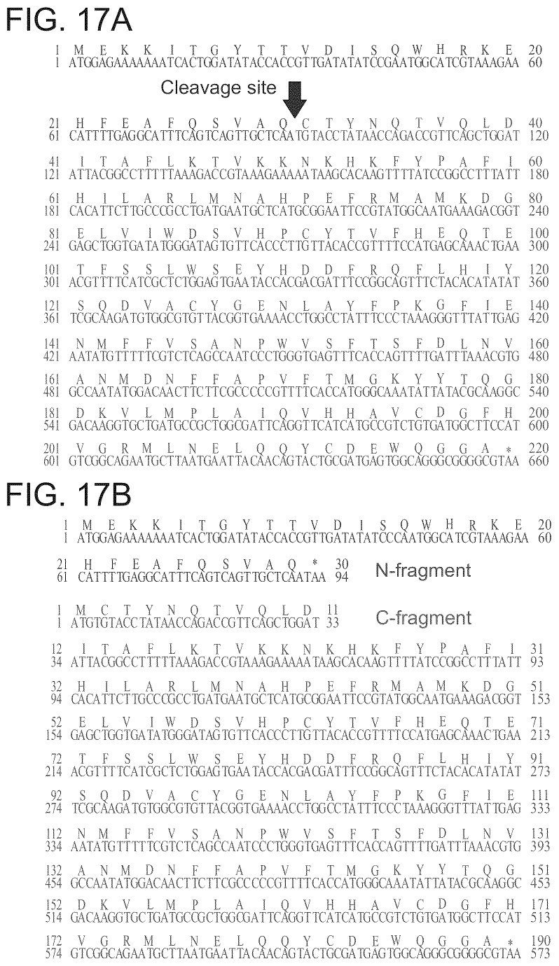

The first fragment of the reporter polypeptide which is linked to ubiquitin may be encoded on the same nucleic acid construct as the second fragment of the reporter polypeptide which is linked to the substrate. Alternatively, the first fragment of the reporter polypeptide which is linked to ubiquitin may be encoded on a different nucleic acid construct as the second fragment of the reporter polypeptide which is linked to the substrate. This embodiment is illustrated in FIG. 1A. Care should be taken when building the constructs such that the expression level of the first fragment is similar to the expression level of the second fragment. Thus for example, the promoter which is used to express the first fragment may be identical to the promoter used to express the second fragment.

In bacterial systems, a number of expression vectors can be advantageously selected depending upon the use intended for the polypeptide expressed. Specifically, the use of different selection markers such as Kan^{R} and Strep^{R}) and different origin of replications (such as ColiE1 and CloDF13) is contemplated.

### Ubiquitinating Enzyme

As used herein, the term “ubiquitinating enzyme” refers to ubiquitin-activating enzymes (E1s), ubiquitin-conjugating enzymes (E2s) or ubiquitin ligases (E3s). Collectively they have the EC number EC 6.3.2.19.

In one embodiment, the ubiquitinating enzyme is a human ubiquitinating enzyme.

Ubiquitin-activating enzymes (E1s) have the EC number EC 6.2.1.45, ubiquitin-conjugating enzymes have the EC number EC 2.3.2.23 and ubiquitin ligases have the EC number 2.3.2.27.

Amino acid sequences of exemplary E1s contemplated by the present invention include, but are not limited to SEQ ID NOs: 9-16.

Amino acid sequences of exemplary E2s contemplated by the present invention are set forth by SEQ ID NOs: 17-36.

Table 1, herein below provides nomenclature and most common synonyms used for E2 ubiquitin conjugating enzymes. The E2 nomenclature is in accordance with that used by the Human Genome Organization.

TABLE 1

| Human Genome | |
|---|---|
| Organization | |
| Nomenclature | Synonym |
| UBE2V2 | UEV2/MMS2 |
| UBE2D1 | UBC4/5/UBCH5A |
| UBE2D2 | UBC4/5/UBCH5B |
| UBE2D4 | HBUCE1 |
| UBE2D3 | UBC4/5 |
| UBE2W | FLJ11011 |
| UBE2B | UBC2/HHR6B/RAD6B/E217K |
| UBE2L6 | RIGB/UBCH8 |
| UBE2N | UBC13 |
| UBE2L3 | UBCH7 |
| UBE2G1 | UBC7/E217K |
| UBE2H | UBC8/E220K |
| UBE2M | UBC12 |
| UBE2F | NCE2 |
| UBE2E2 | UBCH8 |
| UBE2E3 | UBCH9/UBCM2 |
| UBE2S | E224K |
| UBE2U | MGC35130 |
| UBE2R1 | CDC34 |
| UBE2R2 | UBC3B/CDC34B |
| UBE2Z | HOYS7 |
| UBE2J2 | NCUBE2 |
| Probable ubiquitin- | LOC134111/FLJ25076 |
| conjugating enzyme | |
| E2 FLJ25076 | |
| AKTIP | FTS/FT1 |
| UBE2J1 | NCUBE1 |
| UBE2V1 | UEV1/CROC1 |
| UBE2Q2 | DKFZ/UBCI |
| UBE2Q1 | NICE5 |
| | TSG101/VPS23/SG10 |
| UEVLD | UEV3 |

In one embodiment, the ubiquitinating enzyme is an E3 ligase.

Exemplary E3 ligases contemplated by the present invention include, but are not limited to Siah2, Smurf1, MDM2, BRCA1, PARKIN, UBE3A, TRIM5, NEDD4, UBR5,Huwe1, Arkadia, ITCH, MuRF1, TRAF6, Trim32, UBR4, UBE3B and UBE3D.

According to a particular embodiment, the E3 ligase is selected from the group consisting of Siah2, Smurf1, MDM2, BRCA1, PARKIN, UBE3A, UBE3B, MURF1,TRIM32, TRIM5, NEDD4, UBR5 and Huwe1.

In another embodiment the E3 ligase is Siah2, Smurf1, MDM2, BRCA1, PARKIN or UBE3A.

Amino acid sequences of exemplary E3s contemplated by the present invention are set forth in SEQ ID NOs: 37-62.

Below is a brief description of exemplary E3 ligases contemplated by the present invention and some of their exemplary substrates.

### Seven in Absentia Homolog 2 (SIAH2)

SIAH2 is a RING finger type ubiquitin ligases with a catalytic RING domain on its N-terminus, followed by two zinc fingers and a C-terminal substrate binding domain.

Siah2 is an E3 ubiquitin ligase implicated in diverse biological processes including p38/JNK/NF-kB signaling pathways. DNA damage, estrogen signaling, programmed cell death, Ras/Raf pathway, mitosis, and hypoxia.

Siah2 targets numerous substrates for degradation including TRAF2 (ketoglutarate dehydrogenase), Spry2 (Sprouty2), and the prolyl hydroxylase PHD3.

Siah2 also limits its own availability through self-ubiquitination and degradation.

Siah2 play a key role in hypoxia, through the regulation of HIF-1α transcription stability and activity via regulation of PHD3 stability.

### Smad Ubiquitination Regulatory Factor-1 (Smurf1)

Smurf1 is a NEDD4-like Class IV HECT (homologous to E6-AP carboxylterminus) family E3 ligase with catalytic activity.

Smurf1 has been linked to several important biological pathways, including the bone morphogenetic protein pathway, the non-canonical Wnt pathway, and the mitogen-activated protein kinase pathway.

Smurfs possess three functional domains: an N-terminal protein kinase C (PKC)-related C2 domain which binds to phospholipids, targeting Smurfs to intracellular membranes, a central region containing two to four WW (tryptophan residues) protein-interacting domains which mediate ligase-substrate associations through interactions with a variety of proline-rich (PPXY) motifs and proline-containing phosphoserine/phosphothreonine sequences of the protein substrate, and a C-terminal HECT domain, responsible for ubiquitin transfer from a conserved cysteine residue at position 716 to a lysine residue in a substrate protein.

Smurf1 promotes p53 degradation by enhancing the activity of the E3 ligase MDM2. Smurf1 stabilizes MDM2 by enhancing the heterodimerization of MDM2 with MDMX, during which Smurfl interacts with MDM2 and MDMX.

Smurfl is also a key negative regulator of transforming growth factor (TGF)-β/bone morphogenetic protein (BMP) signaling pathway.

### Mouse Double Minute 2 Homolog (MDM2)

MDM2 also known as E3 ubiquitin-protein ligase Mdm2 is an important negative regulator of the p53 tumor suppressor.

Mdm2 protein functions both as an E3 ubiquitin ligase that recognizes the N-terminal trans-activation domain (TAD) of the p53 tumor suppressor and as an inhibitor of p53 transcriptional activation.

Mdm2 contains a C-terminal RING domain (amino acid residues 430-480), which contains a Cis3-His2-Cis3 consensus that coordinates two molecules of zinc. These residues are required for zinc binding, which is essential for proper folding of the RING domain. The RING domain of Mdm2 confers E3 ubiquitin ligase activity and is sufficient for E3 ligase activity in Mdm2 RING autoubiquitination. The RING domain of Mdm2 is unique in that it incorporates a conserved Walker A or P-loop motif characteristic of nucleotide binding proteins, as well as a nucleolar localization sequence.

Mdm2 is capable of auto-polyubiquitination, and in complex with p300, a cooperating E3 ubiquitin ligase, is capable of polyubiquitinating p53. In this manner, Mdm2 and p53 are the members of a negative feedback control loop that keeps the level of p53 low in the absence of p53-stabilizing signals.

### BRCA1

BRCA1-BARD1 constitutes a heterodimeric RING finger complex of the BRCA1/BRCA2-containing complex (BRCC) that contains significant ubiquitin ligase activity.

BRCA1 plays critical roles in the repair of chromosomal damage (error-free repair of DNA double-strand breaks), cell cycle checkpoint control, and genomic stability.

BRCA1 forms several distinct complexes through association with different adaptor proteins, and each complex forms in a mutually exclusive manner.

BRCA1 combines with other tumor suppressors, DNA damage sensors and signal transducers to form a large multi-subunit protein complex known as the BRCA1-associated genome surveillance complex (BASC).

The BRCA1 protein associates with RNA polymerase II, and through the C-terminal domain, also interacts with histone deacetylase complexes. Thus, this protein plays a role in transcription, DNA repair of double-strand breaks ubiquitination, transcriptional regulation as well as other functions

### Parkin

Parkin is a RING-between-RING E3 ligase that functions in the covalent attachment of ubiquitin to specific substrates.

It is best known for regulating the disposal of dysfunctional mitochondria (together with PINK1, a serine threonine kinase) via mitochondrial autophagy (i.e., mitophagy).

Upon loss of mitochondrial membrane potential, PINKI becomes stabilized and activated on the outer mitochondrial membrane (OMM), resulting in recruitment and activation of Parkin.

Parkin facilitates ubiquitination of a broad number of targets expressed on the OMM (e.g., TOM20, Mitofusins, VDAC, Fis1) resulting in recruitment of the autophagy machinery, autophagosome formation and mitochondrial clearance.

In addition to its established role in mitophagy and UPS, Parkin impacts other neuroprotective cellular pathways, including TNFα signaling, and Wnt/β catenin signaling, and is also a putative tumor suppressor.

### UBE3A (Gene Coding for E6-associated protein; E6-AP)

This ligase promotes the ubiquitylation and degradation of p53. E6-AP was subsequently shown to ubiquitylate proteins independent of E6 and to serve an independent secondary function as a transcriptional co-activator of nuclear estrogen receptors. E6-AP has been implicated in a broad range of processes (e.g. cell growth, synaptic formation and function, etc.) and has been shown to have many different target substrates (e.g., HHR23A, CDKN1B, MCM7, etc.).

### Tripartite motif-5 (TRIM5)

This ligase is a RING finger E3 ligase a key anti-viral restriction factor and directly involved in inhibiting HIV-1 replication.

### Neural Precursor Cell Expressed Developmentally Downregulated 4 (NEDD4-1)

Nedd4-1 ubiquitinates a number of substrates, including ENaC, ADRB2, AMPA, Notch, pAKT, VEGFR2, EPS15, LATS1, and MDM2.

The vacuolar protein sorting protein Alix recruits NEDD4 to HIV-1 Gag protein to facilitate HIV-1 release via a mechanism that involves Alix ubiquitination.

NEDD4 also binds and ubiquitinates the latent membrane protein 2A (LMP2A) of the Epstein-Bar virus (EBV) to activate B-cell signal transduction.

### Ubiquitin Protein Ligase E3 Component n-Recognin 5 (UBR5)

This ligase is also known as EDD (E3 identified by Differential Display), EDD1, HHYD, KIAA0896, or DD5.

URB5 acts as a general tumor suppressor by ubiquitinating, which increases p53 levels and induces cell senescence. UBR5 also ubiquitinates TopBP1, a topo-isomerase that intervenes in DNA damage response.

### HUWE1

HUWE1 (also known as ARF-BP1, MULE, LASU1, or HECTH9) is an E3 ligase that regulates the stability of diverse cellular substrates and, in consequence, numerous physiological processes, including DNA replication and damage repair, cell proliferation and differentiation, and apoptosis.

HUWE1 substrates include both tumor promoters (e.g., N-MYC, C-MYC, MCL1) and suppressors (e.g., p53, MYC, MIZ1).

HUWE1 has demonstrated both pro-oncogenic and tumor-suppressor functions in different tumor models.

HUWE1 belongs to the HECT (Homologous to E6AP C-Terminus)-family of ubiquitin E3 ligases.

Other additional E3 ligases and their substrates are provided in Table 2, herein below.

TABLE 2

| Ligase | Substrate | Function |
|---|---|---|
| AMFR | KAI1 | AMFR is also known as gp78. |
| | | AMFR is an integral ER |
| | | membrane protein and |
| | | functions in ER-associated |
| | | degradation (ERAD). AMFR |
| | | has been found to promote |
| | | tumor metastasis through |
| | | ubiquitination of the metastasis |
| | | suppressor, KAI1. |
| APC/Cdc20 | Cyclin B | The anaphase promoting |
| | | complex/cyclosome (APC/C) is |
| | | a multiprotein complex with E3 |
| | | ligase activity that regulates |
| | | cell cycle progression through |
| | | degradation of cyclins and |
| | | other mitotic proteins. APC is |
| | | found in a complex with |
| | | CDC20, CDC27, SPATC1, and |
| | | TUBG1. |
| APC/Cdh1 | Cdc20, | The anaphase promoting |
| | Cyclin B, | complex/cyclosome (APC/C) is |
| | Cyclin A, | a multiprotein complex with E3 |
| | Aurora A, | ligase activity that regulates |
| | Securin, | cell cycle progression through |
| | Skp2, | degradation of cyclins and |
| | Claspin | other mitotic proteins. The |
| | | APC/C-Cdh1 dimeric complex |
| | | is activated during anaphase |
| | | and telophase, and remains |
| | | active until onset of the next S |
| | | phase. |
| C6orf157 | Cyclin B | C6orf157 is also known as |
| | | H10BH. C6orf157 is an E3 |
| | | ubiquitin ligase that has been |
| | | shown to ubiquitinate cyclin B. |
| Cbl | | Cbl-b and c-Cbl are members |
| | | of the Cbl family of adaptor |
| | | proteins that are highly |
| | | expressed in hematopoietic |
| | | cells. Cbl proteins possess E3 |
| | | ubiquitin ligase activity that |
| | | downregulates numerous |
| | | signaling proteins and RTKs in |
| | | several pathways such as |
| | | EGFR, T cell and B cell |
| | | receptors, and integrin |
| | | receptors. Cbl proteins play an |
| | | important role in T cell receptor |
| | | signaling pathways. |
| CBLL1 | CDH1 | CBLL1 is also known as Hakai. |
| | | CBLL1 is an E3 ubiquitin |
| | | ligase that ubiquitinates the |
| | | phosphorylated form of E- |
| | | Cadherin, causing its |
| | | degradation and loss of cell-cell |
| | | adhesions. |
| CHFR | PLK1, | CHFR is an E3 ubiquitin ligase |
| | Aurora A | that functions as a mitotic stress |
| | | checkpoint protein that delays |
| | | entry into mitosis in response to |
| | | stress. CHFR has been shown |
| | | to ubiquitinate and degrade the |
| | | kinases PLK1 and Aurora A. |
| CHIP | HSP70/90, | CHIP is an E3 ubiquitin ligase |
| | iNOS, | that acts as a co-chaperone |
| | Runx1, | protein and interacts with |
| | LRRK2 | several heat shock proteins, |
| | | including HSP70 and HSP90, |
| | | as well as the non-heat shock |
| | | proteins iNOS, Runx1 and |
| | | LRRK2. |
| DTL (Cdt2) | p21 | DTL is an E3 ubiquitin ligase |
| | | that complexes with Cullin4 |
| | | and DDB1, and promotes p21 |
| | | degradation after UV damage. |
| E6-AP | p53, | E6-AP is also known as |
| | Dlg | UBE3A. E6-AP is a HECT |
| | | domain E3 ubiquitin ligase that |
| | | interacts with Hepatitis C virus |
| | | (HCV) core protein and targets |
| | | it for degradation. The HCV |
| | | core protein is central to |
| | | packaging viral DNA and other |
| | | cellular processes. E6-AP also |
| | | interacts with the E6 protein of |
| | | the human papillomavirus types |
| | | 16 and 18, and targets the p53 |
| | | tumor-suppressor protein for |
| | | degradation. |
| HACE1 | | HACE1 is an E3 ubiquitin |
| | | ligase and tumor suppressor. |
| | | Aberrant methylation of |
| | | HACE1 is frequently found in |
| | | Wilms’ tumors and colorectal |
| | | cancer. |
| HECTD1 | | HECTD1 is an ubiquitin E3 |
| | | ligase required for neural tube |
| | | closure and normal |
| | | development of the |
| | | mesenchyme. |
| HECTD2 | | HECTD2 is a probable E3 |
| | | ubiquitin ligase and may act as |
| | | a susceptibility gene for |
| | | neurodegeneration and prion |
| | | disease. |
| HECTD3 | | HECTD3 is a probable E3 |
| | | ubiquitin ligase and may play a |
| | | role in cytoskeletal regulation, |
| | | actin remodeling, and vesicle |
| | | trafficking. |
| HECW1 | DVL1, | HECW1 is also known as |
| | mutant | NEDL1. HECW1 interacts with |
| | SOD1, | p53 and the Wnt signaling |
| | p53 | protein DVL1, and may play a |
| | | role in p53-mediated cell death |
| | | in neurons. |
| HECW2 | p73 | HECW2 is also known as |
| | | NEDL2. HECW2 ubiquitinates |
| | | p73, which is a p53 family |
| | | member. Ubiquitination of p73 |
| | | increases protein stability. |
| HERC2 | RNF8 | HERC2 belongs to a family of |
| | | E3 ubiquitin ligases involved in |
| | | membrane trafficking events. |
| | | HERC2 plays a role in the |
| | | DNA damage response through |
| | | interaction with RNF8. |
| HERC3 | | HERC3 belongs to a family of |
| | | E3 ubiquitin ligases involved in |
| | | membrane trafficking events. |
| | | HERC3 interacts with hPLIC-1 |
| | | and hPLIC-2 and localizes to |
| | | the late endosomes and |
| | | lysosomes. |
| HERC4 | | HERC4 belongs to a family of |
| | | E3 ubiquitin ligases involved in |
| | | membrane trafficking events. |
| | | HERC4 is highly expressed in |
| | | testis and may play a role in |
| | | spermatogenesis. |
| HERC5 | | HERC5 belongs to a family of |
| | | E3 ubiquitin ligases involved in |
| | | membrane trafficking events. |
| | | HERC5 is induced by |
| | | interferon and other pro- |
| | | inflammatory cytokines and |
| | | plays a role in interferon- |
| | | induced ISG15 conjugation |
| | | during the innate immune |
| | | response. |
| HUWE1 | N-Myc, | HUWE1 is also known as |
| | C-Myc, | Mule. HUWE1 is a HECT |
| | p53, | domain E3 ubiquitin ligase that |
| | Mcl-1, | regulates degradation of Mcl-1 |
| | TopBP1 | and therefore regulates DNA |
| | | damage-induced apoptosis. |
| | | HUWE1 also controls neuronal |
| | | differentiation by destabilizing |
| | | N-Myc, and regulates p53- |
| | | dependent and independent |
| | | tumor suppression via ARF. |
| HYD | CHK2 | HYD is also known as EDD or |
| | | UBR5. HYD is a regulator of |
| | | the DNA damage response and |
| | | is overexpressed in many forms |
| | | of cancer. |
| ITCH | MKK4, | ITCH plays a role in T cell |
| | RIP2, | receptor activation and |
| | Foxp3 | signaling through |
| | | ubiquitination of multiple |
| | | proteins including MKK4, |
| | | RIP2 and Foxp3. Loss of ITCH |
| | | function leads to an aberrant |
| | | immune response and T helper |
| | | cell differentiation. |
| LNX1 | NUMB | LNX1 is an E3 ubiquitin ligase |
| | | that plays a role in cell fate |
| | | determination during |
| | | embryogenesis through |
| | | regulation of NUMB, the |
| | | negative regulator of Notch |
| | | signaling. |
| mahogunin | | Mahogunin is an E3 ubiquitin |
| | | ligase involved in melanocortin |
| | | signaling. Loss of mahogunin |
| | | function leads to |
| | | neurodegeneration and loss of |
| | | pigmentation, and may be the |
| | | mechanism of action in prion |
| | | disease. |
| MARCH-I | HLA-DRβ | MARCH1 is an E3 ubiquitin |
| | | ligase found on antigen |
| | | presenting cells (APCs). |
| | | MARCH1 ubiquitinates MHC |
| | | class II proteins and |
| | | downregulates their cell surface |
| | | expression. |
| MARCH-II | | MARCH-II is a member of the |
| | | MARCH family of E3 ubiquitin |
| | | ligases. It associates with |
| | | syntaxin6 in the endosomes and |
| | | helps to regulate vesicle |
| | | trafficking. |
| MARCH-III | | MARCH-III is a member of the |
| | | MARCH family of E3 ubiquitin |
| | | ligases. MARCH-III associates |
| | | with syntaxin6 in the |
| | | endosomes and helps to |
| | | regulate vesicle trafficking. |
| MARCH-IV | MHC class I | MARCH-IV is a member of the |
| | | MARCH family of E3 ubiquitin |
| | | ligases. MARCH-IV |
| | | ubiquitinates MHC class I |
| | | proteins and downregulates |
| | | their cell surface expression. |
| MARCH-VI | | MARCH-VI is also known as |
| | | TEB4 and is a member of the |
| | | MARCH family of E3 ubiquitin |
| | | ligases. It localizes to the |
| | | endoplasmic reticulum and |
| | | participates in ER-associated |
| | | protein degradation. |
| MARCH-VII | gp190 | MARCH-VII is also known as |
| | | axotrophin. MARCH-VII was |
| | | originally identified as a neural |
| | | stem cell gene, but has since |
| | | been shown to play a role in |
| | | LIF signaling in T lymphocytes |
| | | through degradation of the LIF- |
| | | receptor subunit, gp190. |
| MARCH-VIII | B7-2, | MARCH-VIII is also known as |
| | MHC | c-MIR. MARCH-VIII causes |
| | class II | the ubiquitination/degradation |
| | | of B7-2, which is a co- |
| | | stimulatory molecule for |
| | | antigen presentation. MARCH- |
| | | VIII has also been shown to |
| | | ubiquitinate MHC class II |
| | | proteins. |
| MARCH-X | | MARCH-X is also known as |
| | | RNF190. MARCH-X is a |
| | | member of the MARCH family |
| | | of E3 ubiquitin ligases. The |
| | | putative role of MARCH-X is |
| | | not currently known. |
| MDM2 | p53 | MDM2, an E3 ubiquitin ligase |
| | | for p53, plays a central role in |
| | | regulation of the stability of |
| | | p53. Akt-mediated |
| | | phosphorylation of MDM2 at |
| | | Ser166 and Ser186 increases its |
| | | interaction with p300, allowing |
| | | MDM2-mediated ubiquitination |
| | | and degradation of p53. |
| MEKK1 | c-Jun, | MEKK1 is a well known |
| | Erk | protein kinase of the STE11 |
| | | family. MEKK1 phosphorylates |
| | | and activates MKK4/7, which |
| | | in turn activates JNK1/2/3. |
| | | MEKK1 contains a RING |
| | | finger domain and exhibits E3 |
| | | ubiquitin ligase activity toward |
| | | c-Jun and Erk. |
| MIB1 | Delta, | Mindbomb homolog 1 (MIB1) |
| | Jagged | is an E3 ligase that facilitates |
| | | the ubiquitination and |
| | | subsequent endocytosis of the |
| | | Notch ligands, Delta and |
| | | Jagged. |
| MIB2 | Delta, | Mind Bomb 2 (MIB2) is an E3 |
| | Jagged | ligase that positively regulates |
| | | Notch Signaling. MIB2 has |
| | | been shown to play a role in |
| | | myotube differentiation and |
| | | muscle stability. MIB2 |
| | | ubiquitinates NMDAR subunits |
| | | to help regulate synaptic |
| | | plasticity in neurons. |
| MycBP2 | Fbxo45, | MycBP2 is an E3 ubiquitin |
| | TSC2 | ligase also known as PAM. |
| | | MycBP2 associates with |
| | | Fbxo45 to play a role in |
| | | neuronal development. |
| | | MycBP2 also regulates the |
| | | mTOR pathway through |
| | | ubiquitination of TSC2. |
| NEDD4 | | NEDD4 is an E3 ubiquitin |
| | | ligase highly expressed in the |
| | | early mouse embryonic central |
| | | nervous system. NEDD4 |
| | | downregulates both neuronal |
| | | voltage-gated Na+ channels |
| | | (NaVs) and epithelial Na+ |
| | | channels (ENaCs) in response |
| | | to increased intracellular Na+ |
| | | concentrations. |
| NEDD4L | Smad2 | NEDD4L is an E3 ubiquitin |
| | | ligase highly expressed in the |
| | | early mouse embryonic central |
| | | nervous system. NEDD4L has |
| | | been shown to negatively |
| | | regulate TGF-β signaling by |
| | | targeting Smad2 for |
| | | degradation. |
| Parkin | | Parkin is an E3 ubiquitin ligase |
| | | that has been shown to be a key |
| | | regulator of the autophagy |
| | | pathway. Mutations in Parkin |
| | | can lead to Parkinson's |
| | | Disease. |
| PELI1 | TRIP, IRAK | PELI1 is an E3 ubiquitin ligase |
| | | that plays a role in Toll-like |
| | | Receptor (TLR3 and TLR4) |
| | | signaling to NF-κB via the |
| | | TRIP adaptor protein. PELI1 |
| | | has also been shown to |
| | | ubiquitinate IRAK. |
| Pirh2 | TP53 | Pirh2 is also known as RCHY1. |
| | | Pirh2 is a RING domain E3 |
| | | ubiquitin ligase. Pirh2 binds |
| | | p53 and promotes proteosomal |
| | | degradation of p53 independent |
| | | of MDM2. Pirh2 gene |
| | | expression is controlled by p53, |
| | | making this interaction part of |
| | | an autoinhibitory feedback |
| | | loop. |
| PJA1 | ELF | PJA1 is also known as PRAJA. |
| | | PJA1 plays a role in |
| | | downregulating TGF-β |
| | | signaling in gastric cancer via |
| | | ubiquitination of the SMAD4 |
| | | adaptor protein ELF. |
| PJA2 | | PJA2 is an E3 ubiquitin ligase |
| | | found in neuronal synapses. |
| | | The exact role and substrates of |
| | | PJA2 are unclear. |
| RFFL | p53 | RFFL is also known as CARP2 |
| | | and is an E3 ubiquitin ligase |
| | | that inhibits endosome |
| | | recycling. RFFL also degrades |
| | | p53 through stabilization of |
| | | MDM2. |
| RFWD2 | MTA1, | RFWD2 is also known as |
| | p53, | COP1. RFWD2 is an E3 |
| | FoxO1 | ubiquitin ligase that |
| | | ubiquitinates several proteins |
| | | involved in the DNA damage |
| | | response and apoptosis |
| | | including MTA1, p53, and |
| | | FoxO1. |
| Rictor | SGK1 | Rictor interacts with Cullin1- |
| | | Rbx1 to form an E3 ubiquitin |
| | | ligase complex, and promotes |
| | | ubiquitination and degradation |
| | | of SGK1. |
| RNF5 | JAMP, | RNF5 is also known as RMA5. |
| | paxillin | RNF5 plays a role in ER- |
| | | associated degradation of |
| | | misfolded proteins and ER |
| | | stress response through |
| | | ubiquitination of JAMP. RNF5 |
| | | also plays a role in cell motility |
| | | and has been shown to |
| | | ubiquitinate paxillin. |
| RNF8 | H2A, H2AX | RNF8 is a RING domain E3 |
| | | ubiquitin ligase that plays a role |
| | | in the repair of damaged |
| | | chromosomes. RNF8 |
| | | ubiquitinates Histone H2A and |
| | | H2A.X at double-strand breaks |
| | | (DSBs) which recruits 53BP1 |
| | | and BRCA1 repair proteins. |
| RNF19 | SOD1 | RNF19 is also known as |
| | | Dorfin. Accumulation and |
| | | aggregation of mutant SOD1 |
| | | leads to ALS disease. RNF19 |
| | | ubiquitinates mutant SOD1 |
| | | protein, causing a decrease in |
| | | neurotoxicity. |
| RNF190 | | see MARCH-X |
| RNF20 | Histone | RNF20 is also known as BRE1. |
| | H2B | RNF20 is an E3 ubiquitin ligase |
| | | that monoubiquitinates Histone |
| | | H2B. H2B ubiquitination is |
| | | associated with areas of active |
| | | transcription. |
| RNF34 | Caspase-8, | RNF34 is also known as RFI. |
| | -10 | RNF34 inhibits death receptor |
| | | mediated apoptosis through |
| | | ubiquitination/degradation of |
| | | caspase-8 and -10. |
| RNF40 | Histone | RNF40 is also known as BRE1- |
| | H2B | B. RNF40 forms a protein |
| | | complex with RNF20 resulting |
| | | in the ubiquitination of Histone |
| | | H2B. H2B ubiquitination is |
| | | associated with areas of active |
| | | transcription. |
| RNF125 | | RNF125 is also known as |
| | | TRAC-1. RNF125 has been |
| | | shown to positively regulate T |
| | | cell activation. |
| RNF128 | | RNF128 is also known as |
| | | GRAIL. RNF128 promotes T |
| | | cell energy and may play a role |
| | | in actin cytoskeletal |
| | | organization in T cell/APC |
| | | interactions. |
| RNF138 | TCF/LEF | RNF138 is also known as |
| | | NARF. RNF138 is associated |
| | | with Nemo-like Kinase (NLK) |
| | | and suppresses Wnt/β-Catenin |
| | | signaling through |
| | | ubiquitination/degradation of |
| | | TCF/LEF. |
| RNF168 | H2A, | RNF168 is an E3 ubiquitin |
| | H2A.X | ligase that helps protect |
| | | genome integrity by working |
| | | together with RNF8 to |
| | | ubiquitinate Histone H2A and |
| | | H2A.X at DNA double-strand |
| | | breaks (DSB). |
| SCF/β- | IκBα, Wee1, | SCF/β-TrCP is an E3 ubiquitin |
| TrCP | Cdc25A, | ligase complex composed of |
| | β-Catenin | SCF (SKP1-CUL1-F-box |
| | | protein) and the substrate |
| | | recognition component, β-TrCP |
| | | (also known as BTRC). SCF/β- |
| | | TrCP mediates the |
| | | ubiquitination of proteins |
| | | involved in cell cycle |
| | | progression, signal |
| | | transduction, and transcription. |
| | | SCF/β-TrCP also regulates the |
| | | stability of β-catenin and |
| | | participates in Wnt signaling. |
| SCF/FBW7 | Cyclin E, | SCF/FBW7 is an E3 ubiquitin |
| | c-Myc, | ligase complex composed of |
| | c-Jun | SCF (SKP1-CUL1-F-box |
| | | protein) and the substrate |
| | | recognition component, FBW7. |
| | | SCF/FBW7 mediates the |
| | | ubiquitination of proteins |
| | | involved in cell cycle |
| | | progression, signal |
| | | transduction, and transcription. |
| | | Target proteins for SCF/FBW7 |
| | | include the phosphorylated |
| | | forms of c-Myc, Cyclin E, |
| | | Notch intracellular domain |
| | | (NICD), and c-Jun. Defects in |
| | | FBXW7 may be a cause of |
| | | breast cancer. |
| SCF/Skp2 | p27, p21, | SCF/Skp2 is an E3 ubiquitin |
| | Fox01 | ligase complex composed of |
| | | SCF (SKP1-CUL1-F-box |
| | | protein) and the substrate |
| | | recognition component, Skp2. |
| | | SCF/Skp2 mediates the |
| | | ubiquitination of proteins |
| | | involved in cell cycle |
| | | progression (specifically the |
| | | G1/S transition), signal |
| | | transduction and transcription. |
| | | Target proteins for SCF/Skp2 |
| | | include the phosphorylated |
| | | forms of p27Kip1, |
| | | p21Waf1/Cip1, and FoxO1. |
| SHPRH | PCNA | SHPRH is an E3 ubiquitin |
| | | ligase that plays a role in |
| | | DNA replication through |
| | | ubiquitination of PCNA. |
| | | PCNA ubiquitination prevents |
| | | genomic instability from stalled |
| | | replication forks after DNA |
| | | damage. |
| SIAH1 | β-catenin, | SIAH1 is an E3 ubiquitin ligase |
| | Bim, TRB3 | that plays a role in inhibition of |
| | | Wnt signaling through |
| | | ubiquitination of β-catenin. |
| | | SIAH1 has also been shown to |
| | | promote apoptosis through |
| | | upregulation of Bim, and to |
| | | ubiquitinate the signaling |
| | | adaptor protein TRB3. |
| SIAH2 | HIPK2, | SIAH2 is an E3 ubiquitin ligase |
| | PHD1/3 | that plays a role in hypoxia |
| | | through ubiquitination and |
| | | degradation of HIPK2. SIAH2 |
| | | also ubiquitinates PHD1/3, |
| | | which regulates levels of HIF- |
| | | 1α in response to hypoxia. |
| SMURF1 | Smads | SMURF1 is an E3 ubiquitin |
| | | ligase that interacts with BMP |
| | | pathway Smad effectors, |
| | | leading to Smad protein |
| | | ubiquitination and degradation. |
| | | Smurf1 negatively regulates |
| | | osteoblast differentiation and |
| | | bone formation in vivo. |
| SMURF2 | Smads, | SMURF2 is an E3 ubiquitin |
| | Mad2 | ligase that interacts with Smads |
| | | from both the BMP and TGF-β |
| | | pathways. SMURF2 also |
| | | regulates the mitotic spindle |
| | | checkpoint through |
| | | ubiquitination of Mad2. |
| TOPORS | p53, | TOPORS is an E3 ubiquitin |
| | NKX3.1 | ligase and a SUMO ligase. |
| | | TOPORS ubiquitinates and |
| | | sumoylates p53, which |
| | | regulates p53 stability. |
| | | TOPORS has also been shown |
| | | to ubiquitinate the tumor |
| | | suppressor NKX3.1. |
| TRAF6 | NEMO, | TRAF6 is an E3 ubiquitin |
| | Akt1 | ligase that functions as an |
| | | adaptor protein in IL-1R, |
| | | CD40, and TLR signaling. |
| | | TRAF6 promotes NF-κB |
| | | signaling through K63 |
| | | polyubiquitination of IKK, |
| | | resulting in IKK activation. |
| | | TRAF6 has also been shown to |
| | | ubiquitinate Akt1, causing its |
| | | translocation to the cell |
| | | membrane. |
| TRAF7 | | TRAF7 is an E3 ubiquitin |
| | | ligase and SUMO ligase that |
| | | functions as an adaptor protein |
| | | in TNF Receptor and TLR |
| | | signaling. TRAF7 has been |
| | | shown to be capable of self- |
| | | ubiquitination and plays a role |
| | | in apoptosis via MEKK3- |
| | | mediated activation of NF-κB. |
| TRIM63 | Troponin I, | TRIM63 is also known as |
| | MyBP-C, | Murf-1. TRIM63 is a muscle- |
| | MyLC1/2 | specific E3 ubiquitin ligase |
| | | whose expression is |
| | | upregulated during muscle |
| | | atrophy. TRIM63 has been |
| | | shown to ubiquitinate several |
| | | important muscle proteins |
| | | including troponin I, MyBP-C, |
| | | and MyLC1/2. |
| UBE3B | | UBE3B is an E3 ubiquitin |
| | | ligase identified through |
| | | sequence analysis. The specific |
| | | substrates and cellular function |
| | | of UBE3B is currently |
| | | unknown. |
| UBE3C | | UBE3C is an E3 ubiquitin |
| | | ligase also known as KIAA10. |
| | | UBE3C is highly expressed in |
| | | muscle and may interact with |
| | | the transcriptional regulator |
| | | TIP120B. |
| UBR1 | | UBR1 is an E3 ubiquitin ligase |
| | | responsible for proteasomal |
| | | degradation of misfolded |
| | | cytoplasmic proteins. UBR1 |
| | | has also been shown to be a |
| | | ubiquitin ligase of the N-end |
| | | rule proteolytic pathway, which |
| | | regulates degradation of short- |
| | | lived proteins. |
| UBR2 | Histone | UBR2 is an E3 ubiquitin ligase |
| | H2A | that has been shown to |
| | | ubiquitinate histone H2A, |
| | | resulting in transcriptional |
| | | silencing. UBR2 is also part of |
| | | the N-end rule proteolytic |
| | | pathway. |
| UHRF2 | PCNP | UHRF2 is also known as NIRF. |
| | | UHRF2 is a nuclear protein that |
| | | may regulate cell cycle |
| | | progression through association |
| | | with Chk2. UHRF2 also |
| | | ubiquitinates PCNP and has |
| | | been shown to play a role in |
| | | degradation of nuclear |
| | | aggregates containing |
| | | polyglutamine repeats. |
| VHL | HIF-1α | VHL is the substrate |
| | | recognition component of the |
| | | ECV (Elongin B/C, Cullen-2, |
| | | VHL) E3 ubiquitin ligase |
| | | complex responsible for |
| | | degradation of the transcription |
| | | factor HIF-1α. Ubiquitination |
| | | and degradation of HIF-1α |
| | | takes place only during periods |
| | | of normoxia, but not during |
| | | hypoxia, thereby playing a |
| | | central role in the regulation of |
| | | gene expression by oxygen. |
| WWP1 | ErbB4 | WWP1 is an E3 ubiquitin ligase |
| | | commonly found to be |
| | | overexpressed in breast cancer. |
| | | WWP1 has been shown to |
| | | ubiquitinate and degrade |
| | | ErbB4. Interestingly, the |
| | | WWP1 homolog in C. elegans |
| | | was found to increase life |
| | | expectancy in response to |
| | | dietary restriction. |
| WWP2 | Oct-4 | WWP2 is an E3 ubiquitin ligase |
| | | that has been shown to |
| | | ubiquitinate/degrade the stem |
| | | cell pluripotency factor Oct-4. |
| | | WWP2 also ubiquitinates the |
| | | transcription factor EGR2 to |
| | | inhibit activation-induced T cell |
| | | death. |
| ZNRF1 | | ZNRF1 is an E3 ubiquitin |
| | | ligase highly expressed in |
| | | neuronal cells. ZNRF1 is found |
| | | in synaptic vesicle membranes |
| | | and may regulate neuronal |
| | | transmissions and plasticity. |

The term “deubiquitinating” enzyme refers to an enzyme that cleaves ubiquitin from proteins.

According to a specific embodiment, the deubiquitinating enzyme is a cysteine protease or a metalloprotease.

Exemplary deubiquitinating enzymes which may be expressed in the system include USP7 that is known to deubiquitinate MDM2, USP47, USP2, USP7, USP15, USP9X, USP28, USP30.

The ubiquitinating or deubiquitinating enzymes may be expressed from the same expression constructs as the substrate and the ubiquitin or on separate constructs.

### Substrates

Examples of substrates include polypeptides that are known to be ubiquitinated in vivo in humans by E3 ligase or deubiquitinated in vivo by deubiquitinating enzymes.

According to a specific embodiment, the substrate is one that is known to be ubiquitinated differentially in a disease such as cancer.

Exemplary substrates that may be expressed in the bacteria have been described herein above.

According to a particular embodiment, the substrate is selected from the group consisting of PHD3, SPROUTY2, Mitofusin 1, 2, MIRO, NEMO, SMADs, TβR-I, P53, S5A, HHR23, EPHEXIN5, ARC, PPARα, cyclin-B, Cdc25C and Calmodulin.

It will be appreciated that as well as expressing the substrate and the ubiquitin (together with the split reporter polypeptide), the recombinant bacteria should also express the ubiquitinating or deubiquitinating enzyme.

In one embodiment, the bacteria express at least one E1 enzyme, at least one E2 enzyme and at least one E3 enzyme.

Preferably, the bacteria expresses the E2 enzyme that is a cognate pair for the E3enzyme.

In another embodiment, the bacteria expresses at least one deubiquitinating enzyme.

An exemplary system is illustrated in FIG. 1A, whereby the E1 and E2 enzyme are expressed from the same construct as the ubiquitin and the E3 enzyme is expressed from the same construct as the substrate.

In another embodiment, the E1 and E2 enzyme are expressed from the same construct as the substrate and the E3 enzyme is expressed from the same construct as the ubiquitin.

In still a further embodiment, the E1 and E2 enzyme are expressed from the same construct as the substrate and/or the ubiquitin and the E3 enzyme is expressed from an additional expression construct.

The additional expression construct from which the E3-ligase is expressed may use a different selection marker as that used for the other constructs (e.g. Amp^{R} selection marker). It may also use a different origin of replication such as p15A. The promoter for this expression construct may be inducible or constitutive. In one embodiment, the promoter is a weak constitutive promoter such as the pTac promoter which is leaky without the addition of inducer (IPTG).

The present inventors contemplate using a kit for easy preparation of the expression constructs.

Such a kit may comprise:
(i) a first polynucleotide which encodes a first polypeptide fragment which is operably linked to a bacterial regulatory sequence, and a cloning site, wherein a position of the cloning site is selected such that upon insertion of a sequence which encodes a test polypeptide (i.e. the substrate) into the cloning site, following expression in a bacterial cell, a fusion protein is generated which comprises the test polypeptide in frame with the first polypeptide fragment; and
(ii) a second polynucleotide comprising a second nucleic acid sequence encoding a second polypeptide fragment which is attached to ubiquitin, the second nucleic acid sequence being operably linked to a bacterial regulatory sequence, wherein the first polypeptide fragment associates with the second polypeptide fragment to generate a reporter polypeptide (e.g. selectable polypeptide, as further described herein above) dependent on ubiquitination of the test polypeptide.

The first polynucleotide and the second polynucleotide may be on the same expression vector or on a separate expression vector. If present on different expression vectors, then preferably each polynucleotide sequence comprises a bacterial origin of replication.

The kit may comprise a third polynucleotide which encodes at least one ubiquitinating enzyme. Alternatively, the first polynucleotide and/or the second polynucleotide may comprise a sequence which encodes the ubiquitinating enzyme (e.g. E1, E2 and/or E3).

Once an agent has been identified as a regulator of a ubiquitinating or deubiquitinating enzyme, therapeutic potential thereof may be tested using other known in-vitro tests. The candidate agent's therapeutic potential may also be tested in animal models the related disease (e.g. cancer).

Once its therapeutic potential has been corroborated, pharmaceutical compositions comprising same may be synthesized.

It will be appreciated that the system described herein can be manipulated to determine whether an enzyme is capable of ubiquitinating a test substrate.

In this embodiment, the enzyme is expressed in a bacterial cell, as well as the ubiquitin (which is attached to the first polypeptide fragment (as described herein above) and the test substrate (which is attached to the second fragment (as described herein above).

The method may be used to test many different E3 enzymes in various combinations with E2 and E1.

The method proceeds by analyzing for the presence of the reporter polypeptide in the bacterial cell—a presence or amount of the reporter polypeptide is indicative that the enzyme is capable of ubiquitinating the test substrate.

In another aspect, the system described herein can be used to determine whether an enzyme is capable of ubiquitinating a test substrate.

The method of this aspect of the present invention comprises:
(a) expressing a plurality of candidate polypeptide substrates in a bacterial cell population, wherein each of the candidate polypeptide substrates is attached to an identical first polypeptide fragment (as described herein above);
(b) expressing the ubiquitinating enzyme in the bacterial cell population;
(c) expressing ubiquitin in the bacterial cell population, wherein the ubiquitin is attached to a second polypeptide fragment (as described herein above), wherein the second polypeptide fragment associates with the first polypeptide fragment to generate a reporter polypeptide on ubiquitination of the substrate; and
(d) analyzing in bacterial colonies of the bacterial cell population for a presence or absence of the reporter polypeptide, wherein a presence of the reporter polypeptide is indicative of expression of a substrate for the ubiquitinating enzyme.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, biophysical, bacterial genetics and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassay's are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

### Plasmid Construction

Selection system for ubiquitination: Two sets of E. coli-compatible expression vectors were constructed. Ubiquitin and different substrates were fused to two fragments of the murine DHFR, under control of a constitutive lambda phage promoter pLtetO1 (see FIG. 1A).

The pND-Ub vector is based on a modified pZE21 vector, contacting the pLtetO1.The mouse cDNA of the nDHFR fragment (residues 1-108) was PCR-amplified and sub-cloned between the KpnI and PacI sites (the PacI site was inserted into the vector by PCR) into the pZE21 vector. Then a flexible linker was constructed using a two-step PCR method and digested with PacI and NotI (the NotI site was inserted into the vector by PCR) and cloned into the vector downstream to the pLtetO1-nDHFR. Ubiquitination apparatus cassettes containing a His₆-Ub-E2-E1 were PCR-amplified from pGEN plasmids expressing different E2s or Ub mutants and sub-cloned as fusion to the C-terminus of the n/) HFR-linkerl into the NotI and AvrII endonuclease recognition sites.

The pCD-Sub vector was constructed based on a pCDF-duet vector. The T7 promoter was substituted with pLtetO1. The cDHER fragment (residues 109-187) was PCR-amplified and subcloned under the pLtetOl promoter at the MluI and AscI endonuclease recognition sites (the AscI site was inserted by PCR). A second linker was fused to the N-terminus of MBP and cloned into the Ascl site. Substrates were PCR-amplified and sub-cloned into SacII and SpeI sites downstream and in-frame with the c/HFR-linker2-MBP. In some vectors the MBP was removed. Some vectors were prepared by complete chemical synthesis assembly ⁴⁸.

Cloning into expression, purification and detection vectors: The ENTH domain of zebrafish Epn1₍₁₈₋₁₅₇₎ and the dsUIM of human Hrs₍₂₅₇₋₂₇₆₎ were subcloned into the pGST-parallel2 vector between BamHI and EcoRI endonuclease recognition sites. The ENTH domain of yeast Ent1₍₁₋₁₅₂) was subcloned into the pCDF-duet vector fused to His₆-MBP as previously described (pCOG21¹⁷). For detection of ubiquitination in bacteria. yeast Sem1 was subcloned into pCDF-duet vector fused to His₆-MBP. For yeast expression and purification His₆-Sem1 was subcloned into pGREG600 by recombineering that also removed the GFP from the vector.

The structure and sequences of all vectors were confirmed by restriction endonuclease and DNA-sequencing analyses.

Site-directed mutagenesis: Point mutations were introduced using the ExSite approach (Stratagene). The entire vector was amplified using Phusion DNA polymerase. Parental DNA was digested by DpnI and the DNA was blunt ligated. All mutants were sequenced to ensure that the desired mutations were introduced and that no other mutations occurred.

Ubiquitination-dependent E. coli growth assay: E. coli W3110 (from Ezra Yagil laboratory at TAU) were co-transformed with the pND-Ub and the pCD-Sub plasmids and plated on LB agar supplemented with 34 μg/ml kanamycin and 25 μg/ml streptomycin. 5 ml of liquid LB medium supplemented with the same antibiotic concentrations were inoculated with a single colony and left to grow overnight at 37° C. The culture was harvested and washed twice with 5 ml of minimal Davis medium. The optical density (OD₆₀₀ₙₘ) was measured and adjusted to 0.2. Two and a half microliters of the diluted cultures from each sample were spotted on agar Davis plates containing 0, 0.5, 5, 10, 20 or 50 μg/ml of TRIM. The plates were incubated for 2-3 days at 30° C. and photographed with a UV camera in identical conditions. Each spotting assay was repeated at least 6 times.

Selection experiments in solution growth media: Overnight cultures in LB medium (supplemented with 30 μg/ml kanamycin and 25 μg/ml streptomycin) were harvested and re-suspended in Davis minimal medium. Diluted cells (OD₆₀₀, 0.2) were grown at 30° C. in 96-well plates containing 0.2 ml Davis medium supplemented with 0, 1, 5, 7, 10 and 12 μg/ml TRIM and with or without 100 μM Pyr-41. Growth rates were monitored by measuring the optical density (OD₅₉₅) using a microplate spectrophotometer. Doubling time was calculated for early logarithmic growth (OD₅₉₅ between 0.02 and 0.2). All experiments were performed at least 9 times (n=9).

### Genetic Selection Assays for Characterization of Structural Based Mutants

Data collection: E. coli W3110 expressing the pND-Ubs, pCD-Subs (and sometimes also an Ampicillin resistance plasmid that constitutively expresses E3-ligase) grew to logarithmic phase at 37° C. in 5 ml of LB medium supplemented with 23 μg/ml Kanamycin, 16 μg/ml Streptomycin and 33 μg/ml Ampicillin. The culture was harvested and washed once with 5 ml of minimal Davis medium. The bacterial density was adjusted to OD600 nm value of 0.3. Culture samples (2.5 μl each) were spotted on Davis agar Petri dishes containing 10 μg/ml trimethoprim. Culture in each experiment was spotted usually three to four times. Most experiments were repeated at least three to four times (therefore 9<n>16). Time-lapse (30 or 60 minutes) scanning took place in 26° C. incubator using a regular A4/US-letter office scanner (Epson Perfection V37)⁴⁰.

Image analysis: Images were read into Fiji⁴¹ as a stack using ‘import−>image sequence’. The spots density were measured using the Time Series Analyzer V3 (Balaji J 2007; a Java ARchive ImageJ/Fiji plugin that can be downloaded and installed from www(dot)rsb(dot)info(dot)nih(dot)gov/ij/plugins/time-series(dot)html). Regions Of Interest (ROIs) were specified (typically as 20×20 ovals) of and their total intensities (bacterial densities) were integrated and plotted where Z-axis is the image time index. Similarly, the background was measured and calculated and subtracted from the collected data. Logistic regressions of growth curves were calculated using Origin. A single parameter that describes growth efficiency was calculated as follows: the growth curve slope at the ‘half max density’ was extracted and divided by its time index.

Protein purification: Proteins were purified from E. coli using affinity tags as previously described ¹⁷. For crystallization purposes, proteins were concentrated to 5-20 mg/ml using centricon (Amicon Ultra), in a final solution of 50 mM Tris-HCl (pH 8.0), 150 mM NaCl, and 5 mM dithiothreitol (DTT).

Western-blot analysis: Following separation on SDS-PAGE, samples were transferred onto a nitrocellulose membrane and incubated with rabbit anti-His epitope tag antibody (1:20 000 dilution, Rockland) or mouse anti-GST antibody (1:200, Santa Cruz), and infrared dye coupled goat anti-mouse secondary antibody (1:12 000, LI-COR). Blots were scanned using an Odyssey system (LI-COR Biosciences) at 700 and 800 nm.

Crystallization and data collection: Crystallization and data collection and processing of yeast Ent1-ENTH have been reported⁶⁰. Purified Zebrafish Epn1-ENTH was concentrated to 10 mg/ml and was crystallized in 0.1M KPO₄ pH 7.0 and 19% PEG 3,350 at 20° C. Crystals were cryo-protected with 25% ethylene glycol and were frozen in liquid nitrogen. Data were collected at the ID29 beamline (ESRF, Grenoble) and processed with HKL2000 software⁶¹

Structure determination and refinement: Structure of ENTH domains were solved by molecular replacement (MR); 1HOA³⁷ was used for searching model with PHASER ⁶². To facilitate the MR search, the first 17 residues were removed and alanine reduction was exerted to the non-conserved residues. Model building and refinement were carried out with PHENIX ⁶³, Refmac5 ⁶⁴ and COOT⁶⁵.

Ubiquitylated-Rpn10 was purified from E.coli and crystallized as previously described 5,35. It was found that the extremely thin crystals (1-3 μm) were highly sensitive to radiation damage. BEST software was used, which precisely predicted an efficient data-collection strategy to achieve a full completeness dataset for these C2 space-group crystals, with only 95 images at oscillation steps of 1.75°. Data were collected at the ID14-4 beamline (ESRF) at wavelength of λ=0.93930 Å under cryo conditions of 100° K. The structure was determined by molecular replacement using PHASER 42, where the Rpn10-vWA domain from S. pombe (PDB 2X5N) and Ub (PDB IUBQ) 41 were used as initial search models. PHENIX auto-build was used to provide an initial model. Model-building and refinement were carried out with PHENIX 43, Refmac5 44 and COOT 45. The geometry parameters of the isopetide bond were restrained within the refinement process in PHENIX. The structure was validated with PROCHECK 46 and the PDB validation tool. Statistics of Ramachandran analysis yielded 98.7% of the residues were found in the most favored or additional allowed regions and 1.3% were found in the generously allowed regions. None of the residues were found in disallowed regions. It was found that the linker tethering the vWA domain to the UIM has an intrinsic propensity for cleavage at the beginning of the hinge. Indeed, the linker spontaneously clipped-off during crystallization. Consequently, the crystallized protein contained the vWA domain (residues 1-191) and its conjugated Ub at K84.Interestingly, the structures of apo Rpn10 from S. cerevisiae and from S. pombe^{18.24} also present truncated forms of the protein at the same location.

Crosslinking assays: Crosslinking was carried out using 0.5 mM disuccinimidyl substrate (DSS) as described by⁶⁶.

Surface plasmon resonance: Purified His₆-MBP-ENTH or mutants were immobilized on an Ni⁺² chip at a density of ˜800 U. Untagged Ub (analyte) was injected at a flow rate of 20 ml per min in 150 mM NaCl, 10 mM HEPES pH 7.0 and 0.010% poly-sorbate 20 at 24° C.; 500 mM imidazole was used for surface regeneration. To avoid immobilization of aggregated analyte the purified Ub was chromatographed by gel filtration and briefly sonicated immediately before SPR experiments. Data were processed with BioEvaluation and fitting carried out as a single-site-binding model with OriginLab. Standard errors derived from at least three samples.

SPR analysis of Rpn10-vWA:Ub and mutants non-covalent interactions. The experimental set-up comprised α-GST antibody immobilized on a CM5 chip according to the manufacturer's protocol (GE Healthcare). The ligands (Rpn10 derivatives) were expressed and purified as GST-fusion-proteins and captured on the chip. Free mono-Ub was the analyte. Prior to each experiment the ligand and the analyte proteins were subjected to size exclusion chromatography. Each measurement was taken in triplicate. The experiment comprised 90-100 seconds for binding, 300-350 s for dissociation. Wild-type and mutant Ub analytes were injected at a flow rate of 10 ml/min in 10 mM HEPES pH 7.0. 150 mM NaCl, and 0.005% polysorbate-20 at 25 C. At the end of each experiment, the ligands were removed and surface regeneration was achieved by flowing 10 mM glycine-HCl pH 2.6, followed by sequential washing steps of 1-2 min with 0.1% SDS, 10 mM NaOH. Data were processed using Biacore BIAevaluation software. A single-site-binding model was used for curve fitting of the binding data (Sigma Plot). For plotting. data were scaled such that Rmax=100.

Detection of ubiquitination in vivo: pScHis₆-Sem1 plasmid was transformed into the SEY6210, rsp5::HIS3, pDsRed415-rsp5^{WT} and rsp5-1 (rsp5^{L7338}) MATa ura3-52, his3-200, trp1-901, lys2-801, suc2 9, leu2-3 strains and grew at 26 in 50 ml of YPD medium (2% glucose) supplemented with 200 μg/ml G418. At log phase the cultures were harvested and the pastes were washed with DDW and transferred to 50 ml YPD medium (2% galactose) supplemented with 200 μg/ml G418. Each culture was divided into two flasks and grew at permissive (26° C.) and restrictive (37° C.) temperatures for additional 4 hours. The cultures were harvested and lysed in 2 ml cold 1.85 N NaOH, 7.5% β-mercaptoethano for 10 min on ice. Proteins were precipitated with the addition of half volume of 50% TCA (final concentration of 25% TCA) and collected by centrifugation (18,000 rpm 20 min). The pellet was resuspended and washed with 3 ml ice-cold 80% acetone, and collected by centrifugation (18,000 rpm 5 min). Then the pellet was re-suspended in 1.5 ml resuspension buffer (6 M guanidine HCl, 100 mM Tris-HCl, 100 mM NaCl, 0.1% triton X-100, pH 8.8) and incubated at 25° C. for 1 hour while rotating. The protein fraction incubated 20 min at 4° C. with 70 μl Ni+2 resin and washed twice with resuspension buffer followed by two washes with buffer 2 (8M urea, 100 mM Tris-HCl pH 8.8, 100 mM NaCl, 0.1% triton X-100) and two washes with buffer 3 (50 mM Tris-HCl pH 7.5, 150 mM NaCl). The Ni+2 beads were boiled in Laemmli buffer and separated by SDS-PAGE followed by western blot analysis with anti-His6 antibody.

Library construction: Yeast GST-tagged ORFs (GE collection) were pooled from 384 pins plated cultures and plasmids were isolated as a pool. The GST fusion genes were PCR amplified. The PCR products were size fractionated (350 to 3000 bp) by gel electrophoresis followed by PCR purification using PureLink, kit (Invitrogen) and sub-cloned in-frame with cDHFR to the pCD-Sub vector by recombineering. The resulted library was transformed to DH5α cells and kept in −80° C. Isolated plasmids of the library were transformed to W3110 competent cells that contained the pND-Ub and constitutively expressed E3-ligase plasmids. Followed transformation the bacteria were plated on selective media.

Accession codes: Atomic coordinates and structure factors for the crystal structures of Saccharomyces cerevisiae Ent1-ENTH and Danio rerio Epn1-ENTH domains have been deposited in the Protein Data Bank under ID codes rcsb079376 and rcsb074441 respectively.

### Results

### Construction of a Selection System for Ubiquitination Events in Bacteria

A system was generated for genetic screening of ubiquitination events in E. coli. In this system, two fragments of a split reporter gene are tethered to Ub and a ubiquitination substrate and are co-expressed along with ubiquitination apparatus in E. coli (FIG. 1A). Specifically, the N-terminal fragment of murine dihydrofolate reductase (nDHFR) was fused to the N-terminus of Ub (in a plasmid denoted pND-Ub) and the C-terminal fragment of DHFR (cDHFR) to the N-terminus of the substrate (in a plasmid denoted pCD-Sub). Pending on substrate ubiquitination, the two DHFR fragments are assembled into a functional enzyme conferring antibiotic [trimethoprim (TRIM)] resistance and growth on selective media¹⁹. The DHFR fragments were tethered to Ub and to the substrate with long linkers designed to confer flexible but stable characteristics facilitating the functional assembly of the reporter (FIG. 9). The system has a polycistronic architecture in which ubiquitination apparatus and the substrate are co-expressed from two or three compatible vectors (FIG. 1A). Synthetic operons are expressed from a constitutive promoter [an unregulated λ-phage left promoter (pL)], suitable for bacterial genetic studies.

To assess the functionality of the system, the well-characterized Ub-receptor Vps9¹⁵⁻¹⁷ was fused to cDHFR (pCD-Sub) and co-expressed together with Rsp5 (E3) and the pND-Ub vector that expresses E1 and yeast Ubc4 (E2). It was found that the W3110strain provided the best genetic background under the described experimental conditions. Bacteria expressing a complete ubiquitination system for Vps9 grew under both permissive and non-permissive conditions (FIG. 1B). However, strains lacking Ub or E1/E2 did not grow on selective media. To demonstrate that the developed system maintains the known E3:substrate specificity. Rsp5 was replaced with Siah2, a non-cognate E3-ligase. Bacteria grew only when the cognate E3 was expressed (FIG. 1C), even though Siah2 was functional in the selection system, as it promoted self-ubiquitination (FIG. 2B).

The split DHER system was originally developed for identification of non-covalent protein-protein interactions¹⁹, suggesting that the selection system may detect non-covalent UBDs:Ub interactions. Since UBDs present a fairly low affinity to Ub¹⁴, different antibiotic concentrations were tested to identify suitable growth conditions for Vps9:Ub non-covalent interactions. Although Vps9 presents one of the highest UBD:Ub measured affinities^{15, 16}. growth was found only at antibiotic concentration of 0.1 μg/ml (FIG. 1D). Since such antibiotic concentration is limited for selection, it seems that the developed system is selective only for ubiquitination events.

### The Ubiquitination Selection System Facilitates Identification and Characterization of E3s

Most E3-ligases undergo self-ubiquitination²⁰. The developed selection system thus provides a straightforward tool for their identification and characterization as self-ubiquitination is predicted to confer antibiotic resistance. Yeast Rsp5 and human Siah2, representatives of the HECT and the RING ligases, respectively, were fused to cDHER. Growth phenotypes were observed under non-permissive conditions, only when all the required ubiquitination components were co-expressed (FIGS. 2A-2B).

Siah2 inhibition by menadione may play an important role in cancer therapy²¹. As the engineered bacteria became addicted to self-ubiquitination of Siah2. its inhibition was predicted to present growth arrest phenotype. FIG. 2B (right) shows a growth arrest phenotype in the presence of menadione only under the addictive conditions. Similarly. Cdc34, a non-cognate E2, did not support growth. The developed system could thus be employed for screening of potential drugs. It was suggested that some E3s use one E2 to attach the first Ub and a different E2 for building the Ub-chain²². The present system may facilitate the identification of such E2s.

### Identification of a Novel E3-Ligase and its Cognate E2s

To demonstrate the system's ability to identify a novel E3-ligase, the present inventors focused on a U-box family from the pathogenic enterohaemorrhagic E. coli (EHEC). They used the well-characterized human ligase CHIP as a probe in a PSI-BLAST search against the EHEC proteome. The search retrieved an uncharacterized sequence, ECs3488, as a potential ligase containing a conserved 35-amino-acid sequence with 22% identity to a helix-loop-helix-beta region of the CHIP U-box domain²³ (FIG. 2C). ECs3488, also named NleG6-3. has been postulated as an E3-ligase, but its expression and function have never been demonstrated^{24, 25}. ECs3488 was cloned as a fusion with cDHER and screened against a full yeast library of E2s, resulting in the identification of Ubc4/5 as cognate E2s for the putative E3-ligase (FIG. 2D). Then, the ligase functionality was examined with the human E2 orthologs UbcH5B/C (FIG. 2E).

Based on the structures of NleG2-3 and of the CHIP:UbcH5a complex (PDB 2KKX and 20XQ)²⁴ structural model for the ECs3488:E2 interface was built (FIG. 2F) and the selection system was used to assess the model. The results corroborated the structural model, as the ECs3488:E2-binding mutants presented significant growth arrest phenotypes (FIG. 2G).

### Selection Approach for Identification and Characterization of UBDs

Ubiquitin binding domains (UBDs) usually bind mono-Ub with low affinity, ranging from 2 μM to 2100 μM^{15, 26}, posing a challenge to biochemical and biophysical studies. The selection system stabilizes the dynamic and weak UBD:Ub non-covalent interactions by forming a covalent bond between Ub and Ub-receptor (i.e. ubiquitination) in the bacteria. The present inventors assessed the ability of the system to sense low affinity UBD by tethering the double-sided UIM (dsUIM) of Hrs as substrate^{27, 28} (FIG. 3A). The structures of Hrs:STAM (also known as ESCRT-0 complex) and particularly its dsUIM:Ub complex, were determined and facilitated detailed molecular assessment^{29, 30}. The E3-independent self-ubiquitination of most Ub-receptors and the promiscuous function of Ubc4/5 subfamily members aided in the analysis. Available lysine residues for ubiquitination do not necessarily need to be part of the UBD as was demonstrated for several UIM and UBA proteins³¹. Alanine residues at each face of the dsUIM were demonstrated to interact with the Ub I44 hydrophobic patch (FIG. 3B). Indeed, it was found that the A266Q, A268Q double mutant presented a growth arrest phenotype. Similarly. 144-patch mutant abolished bacterial growth. The phenotype of Ub-G76R demonstrated that growth is also dependent on ubiquitination. Furthermore. Western-blot analysis showed that a covalent bond between Ub and Hrs is generated (FIG. 3C). Finally, it was demonstrated that the growth is dependent on a functional ubiquitination apparatus by omitting the E1/E2 enzymes (FIG. 3A) or by administration of the E1 inhibitor, PYR-41³² (FIG. 3D).

The system's functionality with other structurally different UBDs, including the proteasomal receptor Rpn10, human STAM1-UIM and ALIX-V domains³³ and the yeast Hse1-VHS domain²⁶, that together are involved in multivesicular and retroviruses budding, was furthered verified/validated (FIGS. 3E-3H).

Similar to ESCRT-0 (STAM, HRS and Hse1) components, GGAs proteins also utilize UBDs to transport ubiquitylated-cargo from Golgi to the multivesicular body. An affinity of 2100 μM was demonstrated to the GGA3-VHS:Ub complex²⁶. Critical tryptophan and leucine residues were identified in STAM1 and other VHS domains that bind Ub at the 144 patch. Moreover, the VHS domains of GGA1 and 2. which naturally lack the critical leucine, do not bind Ub. It was demonstrated that despite the weak affinity, the selection system distinguished between these phenotypes (FIG. 3I).

One benefit of a bacteriostatic antibiotic like trimethoprim is that it enables the accumulation of functional DHER assemblies, due to the ubiquitination, up-to a threshold level that is sufficient to confer resistance while not harming the bacteria. Thus, the developed system may provide a super-sensitive readout for genetic identification and characterization of potential UBDs, without the need to purify them.

### ENTH is a UBD

The present inventors next sought to challenge the system to detect a novel ultraweak-affinity UBD. ENTH domains assume a similar fold to that of VHS³⁴. Moreover, Epsin proteins possess a similar architecture to the Ub-receptors Hrs, STAM and GGAs by harboring VHS/ENTH, two Ub binding patches (dsUIM or 2xUIM or GAT domains) followed by a long flexible linker containing endocytic machinery binding elements including a clathrin-binding box (FIG. 10). Therefore, although ENTH probably lacks the critical tryptophan or leucine residues²⁶, it was speculated that it too binds Ub. The selection system was employed and it was demonstrated that cDHFR-ENTH domains of yeast and zebrafish promote growth on selective media in an E3-independent manner (FIGS. 4A-4B). This suggests that these ENTH domains directly bind Ub˜E2. Indeed, biochemical crosslinking assays and purification/detection of ubiquitylated yeast Entl derivatives from E. coli¹⁷ strongly support the genetic data suggesting that ENTH directly binds Ub (FIGS. 11A-11E).

### Structure of ENTH Domain Provides Insight Into Mechanism of Ub Recognition

To obtain high resolution information on the ENTH:Ub interaction, the present inventors tried to crystalize the complex. Probably due to the ultraweak affinity (see quantification below), only crystals of apo ENTH or Ub were obtained. The the structures of yeast (Sc_ENTH) and zebrafish (Zf_ENTH) domains were determined by molecular replacement to 1.95 Å and 1.41 Å resolutions respectively (Table 3 and FIGS. 4C-4J).

TABLE 3
Crystallographic Data and Refinement Statistics

| Construct | Z.ƒ ENTH | | S.c ENTH | |
|---|---|---|---|---|
| Data Collection | | | | |
| X-ray source | ESRF ID29 | | ESRF ID29 | |
| Wavelength (Å) | 0.978 | | 0.978 | |
| Space group | P1 | | P 21 21 21 | |
| Cell dimensions | | | | |
| a, b, c, (Å) | a = 42.364, | | a = 32.69, | |
| | b = 51.724, | | b = 35.46, | |
| | c = 64.639 | | c = 110.62 | |
| α, β, γ (°) | α = 89.333, | | α = β = γ = 90 | |
| | β = 76.373, | | | |
| | γ = 79.737 | | | |
| Resolution (Å) | 1.4-62.79 | (1.409-1.425) ^{a} | | |
| R-merge^{¶} (%) | 7.5 | (3.2) | 0.166 | |
| I/σI | 5.3 | (4.4) | 6.79 | (1.50) |
| Completeness (%) | 94.0 | (88.97) | 99.18 | (99.58) |
| Redundancy | 3.4 | (3.0) | | |
| Observed | 325,868 | | 37572 | |
| reflections | | | | |
| Unique | 95136 | (9256) | 9806 | (1429) |
| reflections | | | | |
| Refinement | | | | |
| Resolution | 1.41-62.79 ^{a} | (1.41-1.425) | 1.95-55.42 ^{a} | (1.95-2.0) |

| | | |
|---|---|---|
| R_{work}/R_{free} | 0.1545/0.2015 | 0.1862/0.2364 |
| Number of atoms | 6022 | 1254 |
| Protein | 5189 | 137 |
| Ligand/ion | 10 | |
| Water | 823 | 97 |
| average B factor | 17.5 | 19.00 |
| (A²) | | |
| Solvent (%) | 28.83 | 29.40 |
| r.m.s. deviations | | |
| Bond lengths (Å) | 0.009 | 0.018 |
| Bond angles (°) | 1.134 | 1.30 |
| Ramachandran | | |
| (%) | | |
| Favored | 94.1 | 96 |
| Additional | 5.7 | 4.0 |
| allowed | | |
| Outliers | 0.2 | 0.0 |
| Disallowed | 0.0 | 0.0 |
| PDB code | 5LP0 | 5LOZ |

^{¶}R-merge = Σhkl Σi | Ii(hkl) − (I(hkl)) | /Σhkl Σi Ii(hkl), where Ii(hkl) is the intensity of the ith observation of reflection hkl and <I(hkl)> is the average intensity of reflection hkl.
^{a} Outer resolution shell

High-quality electron density maps (FIGS. 4C-4D) showed that although these structures are highly similar, apparent differences can be seen in the loop tethering helices-3 and 4 and the angle between the superhelix structure and helix-8 (FIGS. 12A-12B).

To generate a structural model of the ENTH:Ub complex, the ENTH domains were superimposed onto the STAM1-VHS:Ub complex^{26, 35, 36}. The models showed that the STAM1 W26 and L30 Ub-binding residues were naturally substituted with S37 and S41 in Zf_ENTH and K36 and 140 in Sc_ENTH. (FIGS. 4C-4J). Intriguingly, these models suggest that Ub R42 and R72 form electrostatic interactions with E42 and D45 or E41 and E44 of Zf_ENTH and Sc_ENTH, respectively. Both models predicted additional interactions that seemed to contribute little to the binding.

Intriguingly, superimposing the ENTH:Ub model onto the structure of ENTH complex with the membrane lipid phosphatidylinositol-4,5-bisphosphate³⁷ shows that ENTH binds Ub and the membrane lipid at opposite sites (FIG. 13), suggesting that ENTH can bind Ub while associated with the membrane. Similarly, superimposing the VHS:Ub complex onto the VHS:M6PR-tail (the acidic-cluster-dileucine sorting signal of the mannose-6-phosphate receptor) complex^{38, 39}, shows the same phenomenon, suggesting that both VHS and ENTH domains can recognize ubiquitylated-ubiquitylated-transmembrane-cargo while associated with the membrane.

### Genetic Approach for Structural Validation of the ENTH:Ub Interface

ENTH domains lack the critical Trp or Ile, suggesting that cumulative interactions of peripheral residues compensate for its role in Ub binding. To evaluate the contributions of specific residues to the ENTH:Ub interaction, point mutations were introduced at the predicted interface. The growth rates were monitored by time-lapse scanning the bacteria using a simple A4/US-letter scanner with a modified control software⁴⁰. To quantify the growth rates, a simple Fiji⁴¹ based time series analyzer procedure was applied to measure a typical stack of 100-200 scans (see details in the Materials and Methods section) by means of optical density (FIGS. 5B-5F).

Ala mutagenesis or exchanging the charge of the acidic residues of Zf_ENTH demonstrated growth arrest phenotypes (FIGS. 5A,5C,5D). A similar phenotype, though less severe for the Ub R42E, R72E mutant, was found when the acidic residues of the yeast protein were mutated, suggesting slight structural differences between these complexes. Remarkably, a permutation cycle in which the Zf_ENTH E42 and D45 residues were replaced with arginine residues, and Ub R42 and R72 were replaced with glutamic residues, restored growth (FIG. 5D). This result reflects the accuracy level of the structural model and the high sensitivity of the selection system. Moreover, the Ub R42E, R72E mutant could not suppress the Zf_ENTH E42A, D45A mutant, signifying the importance of these electrostatic interactions. The Zf ENTH S37G and Y83A mutants, predicted to provide lesser contributions to the binding, indeed yielded minor but significant growth phenotypes (FIG. 5A). Taken together, the results of this mutational analysis strongly corroborate the structural model and demonstrate the power of the developed system to detect and quantify relatively minor differences in protein-protein interactions along Ub pathways.

The yields of ubiquitination of the Sc_ENTH wild-type and mutant proteins were biochemically quantified (FIGS. 5G-5H). The ENTH E41R, E44R and the Ub R42E. R72E double mutants significantly reduced the ubiquitination yield by about 60-80%.

Finally, to biophysically corroborate the data and to quantify the affinity of the ENTH:Ub complex, Surface Plasmon Resonance (SPR) measurements were performed with immobilized Sc_ENTH and free mono-Ub (FIG. 5I). Ultraweak binding with K_{d} of 2,300 μM was found for the wild-type complex. This result is compatible with our model and previous measurements of homologous VHS:Ub complexes in Hrs. Vps27 and GGA3, which present affinities of 1,400, 1,500 and 2,100 μM, respectively²⁶. Interestingly, the Ub R42E, R72E or Sc_ENTH E41R. E44R mutants showed saturation binding curves that could be fit to a single binding model (FIG. 5I and FIGS. 14A-14C) with respective estimated affinities 2.6 fold and 3.5 fold lower than that of the wild-type. Notably, for these mutants, the Ub (analyte) concentrations were too low to obtain accurate K_{d} values, as reflected in the high standard errors. Together, the correlation between the SPR measurements, the genetic and biochemical data provides a rough estimation for the sensitivity of the selection system in monitoring Ub binding.

Ultraweak (˜3,000 μM) protein-protein interactions are significant as they regulate various biological functions^{42, 43}. Typically, K63-tri-Ub chains constitute the main signal for clathrin-dependent membrane protein trafficking⁴⁴⁻⁴⁶. Therefore, Ub-receptors decoding this signal usually possess three Ub-binding patches⁴⁷ (FIGS. 11A-11E). Avidity and/or cooperative of tandem UBDs render selectivity of these Ub-receptors. The contribution of the VHS domain to the total affinity and selectivity of VHS-UIM proteins has been thoroughly studied^{26, 35}. Therefore, the ultraweak affinity described herein should be considered in the context of full-length Epsin, which contains two additional UIMs.

### Identification of Sem1 as Ubiquitination Target of Rsp5

One of the greatest challenges in the Ub field is to identify association between E3-ligases and their cognate ubiquitination targets (FIGS. 6A-6D). To demonstrate the potential of the developed system to address this challenge, the present inventors constructed a whole genome yeast fusion library in-frame with cDHFR and screened the library against Rsp5. The entire array of yeast GST-tagged ORFs (GE collection) were amalgamated and plasmids were isolated as a pool. The plasmid library was PCR-amplified and the products were subcloned into the developed selection system by Gibson assembly48. As many Ub-receptors may undergo E3-independent ubiquitination, the present inventors expected to obtain false positive growth of these ORFs, and therefore they employed an assay for E3-independent ubiquitination. They compared the growth rates of the positive colonies with and without Rsp5 using the scanner as described above. Less than 100 positive colonies were identified (from 10 Petri dishes). Most of them showed very similar growth rates in the presence or absence of Rsp5. However, the colony of Sem1 demonstrated significantly higher growth rates when Rsp5 was co-expressed. Sem1 and its human orthologue DSS1 (Deleted Split-hand/Split-foot) are involved in critical processes including development, proteasome assembly. DNA repair and cancer⁴⁹⁻⁵³. As DSS1 possesses two conserved UBDs⁵⁴, Sem1 was tested to see if it underwent E3-independet ubiquitination. It was found that Rsp5 significantly promoted Sem1 ubiquitination (FIGS. 6A-6B). Moreover, detection of ubiquitylated His6-MBP-Sem1 from E. coli showed highly similar results (FIG. 6C). Since this is the first report for Sem1 ubiquitination, the present inventors tested if Sem1 undergoes Rsp5 dependent ubiquitination in vivo in yeast. Ubiquitination was detected in yeast extracts of wild-type and a temperature sensitive rsp5 allele, rsp5-1. Expression of galactose dependent His₆-Sem1 at permissive and restrictive temperatures showed that Rsp5 is a bona-fide ligase of Sem1, as unlike the wt, the ubiquitination in rsp5-1 was detected at 26° C. but not at 37° C. (FIG. 6D).

### Use of Chloramphenicol Acetyl Transferase (CAT) as a Selection Marker

Based on the crystal structure of CAT_{I} in its apo and complex with chloramphenicol (PDB accessions 3U9B and 3U9F) the present inventors designed a split-CAT system. To test if the newly designed split-CAT system can identify ubiquitination events, they tethered a ubiquitylation target to the nCAT fragment and Ub to the cCAT fragment (FIGS. 15A-15D). The fused proteins were co-expressed with their cognate ubiquitylation apparatus and bacteria were spotted on selective media (rich agar supplemented with 7-10 mg of chloramphenicol per ml). At first, a simple ubiquitylation cascade was tested consisting of Ub-receptor (a Ub-Binding Domain UBD containing protein) as the ubiquitylation target. Specifically. the Ub-receptor of Hse1-VHS domain tethered to the nCAT was co-expressed with nCAT tethered to Ub. Wheat E1 (Uba1) and the yeast E2 (Ubc4) were also expressed. As shown in FIG. 16A expression of the complete ubiquitylation cascade of VHS domain tethered to the split-CAT system presented growth phenotype. However, when the ubiquity lation enzymes E1and E2 or Ub were removed growth arrest phenotypes were found.

To compare the growth efficiency between the split-DHFR vs. the split-CAT systems, a UBE3A (also known as E6AP) Rpn10 ubiquitylation dependent cascade was constructed in both systems. A K>R mutation within UBE3A was constructed which results in a constitutive hyperactive E3-ligase. This ligase was expressed from a 3^{rd} vector under the regulation of the leaky promoter pTac (i.e. without the addition of IPTG) in bacteria that express Ub, Rpn10, Uba1 (E1) and UBCH7 (E2). FIG. 16B shows a significant difference in growth efficiency between the two systems.

To demonstrate the system application to study the effect of point mutations an Angelman syndrome (AS) mutation was introduced into UBE3A and bacterial growth was analyzed in wild-type (self-arrested), K>R mutant (hyperactive) and AS mutation on the background of the K>R mutation (FIG. 16C). It was found that the K>R mutation resulted in a higher efficient growth phenotype while the AS mutation resulted in a decreased efficient growth phenotype compared with the WT enzyme. Similarly, the function of UBE3B a HECT E3 Ub-ligase that presents a difficulty in purification in its active from and which is involved in Kaufman Syndrome (KS; Flex et al. 2013) was also assessed in the system. Using the selection system, the critical lysine residue that undergoes self-ubiquitylation that lead to allosteric restrain of the enzyme was identified and a K>R unrestrained mutant was constructed. KS mutation on the background of the UBE3B K>R demonstrated a growth phenotype (FIG. 16D).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

1. Weissman. A. M., Shabek, N. & Ciechanover. A. The predator becomes the prey: regulating the ubiquitin system by ubiquity lation and degradation. Nature reviews. Molecular cell biology 12, 605-620 (2011).
2. Wild. P. et al. Phosphory lation of the autophagy receptor optineurin restricts Salmonella growth. Science 333, 228-233 (2011).
3. Nalepa, G., Rolfe. M. & Harper. J. W. Drug discovery in the ubiquitin-proteasome system. Nat Rev Drug Discov 5, 596-613 (2006).
4. Mizushima, N., Levine, B., Cuervo, A. M. & Klionsky, D. J. Autophagy fights disease through cellular self-digestion. Nature 451, 1069-1075 (2008).
5. Ciechanover, A. & Brundin, P. The ubiquitin proteasome system in neurodegenerative diseases: sometimes the chicken, sometimes the egg. Neuron 40, 427-446 (2003).
6. Bachmair, A., Finley, D. & Varshavsky. A. In vivo half-life of a protein is a function of its amino-terminal residue. Science 234, 179-186 (1986).
7. Deshaies, R. J. & Joazeiro, C. A. RING domain E3 ubiquitin ligases. Annu Rev Biochem 78, 399-434 (2009).
8. Hershko, A., Ciechanover, A., Heller, H., Haas, L. A. & Rose, A. I. Proposed role of ATP in protein breakdown: Conjugation of proteins with multiple chains of the polypeptide of ATP-dependent proteolysis. Proc Natl Acad Sci U S A 77, 1783-1786 (1980).
9. Li, W. et al. Genome-wide and functional annotation of human E3 ubiquitin ligases identifies MULAN, a mitochondrial E3 that regulates the organelle's dynamics and signaling. PLoS One 3, e1487 (2008).
10. Wilkinson, K. D., Urban, M. K. & Haas, A. L. Ubiquitin is the ATP-dependent proteolysis factor I of rabbit reticulocytes. J Biol Chem 255, 7529-7532 (1980).
11. Hicke, L., Schubert, H. L. & Hill, C. P. Ubiquitin-binding domains. Nat Rev Mol Cell Biol 6, 610-621 (2005).
12. Di Fiore, P. P., Polo, S. & Hofmann, K. When ubiquitin meets ubiquitin receptors: a signalling connection. Nat Rev Mol Cell Biol 4, 491-497 (2003).
13. Hoeller, D. et al. Regulation of ubiquitin-binding proteins by monoubiquitination. Nat Cell Biol 8, 163-169 (2006).
14. Hurley, J. H., Lee, S. & Prag, G. Ubiquitin-binding domains. Biochem J 399, 361-372 (2006).
15. Prag, G. et al. Mechanism of ubiquitin recognition by the CUE domain of Vps9p. Cell 113, 609-620 (2003).
16. Shih, S. C. et al. A ubiquitin-binding motif required for intramolecular monoubiquity lation, the CUE domain. EMBO J 22, 1273-1281 (2003).
17. Keren-Kaplan, T. et al. Synthetic biology approach to reconstituting the ubiquity lation cascade in bacteria. EMBO J 31, 378-390 (2012).
18. Keren-Kaplan, T. & Prag, G. Purification and crystallization of mono-ubiquitylated ubiquitin receptor Rpn10. Acta crystallographica. Section F, Structural biology and crystallization communications 68, 1120-1123 (2012).
19. van den Bogaart, G. et al. Synaptotagmin-1 may be a distance regulator acting upstream of SNARE nucleation. Nat Struct Mol Biol 18, 805-812 (2011).
20. Lorick, K. L. et al. RING fingers mediate ubiquitin-conjugating enzyme (E2)-dependent ubiquitination. Proc Natl Acad Sci U S A 96, 11364-11369 (1999).
21. Shah, M. et al. Inhibition of Siah2 ubiquitin ligase by vitamin K3 (menadione) attenuates hypoxia and MAPK signaling and blocks melanoma tumorigenesis. Pigment cell & melanoma research 22, 799-808 (2009).
22. Windheim, M., Peggie, M. & Cohen, P. Two different classes of E2 ubiquitin-conjugating enzymes are required for the mono-ubiquitination of proteins and elongation by polyubiquitin chains with a specific topology. Biochem J 409, 723-729 (2008).
23. Bhavsar, A. P., Guttman, J. A. & Finlay, B. B. Manipulation of host-cell pathways by bacterial pathogens. Nature 449, 827-834 (2007).
24. Wu, B. et al. N1eG Type 3 effectors from enterohaemorrhagic Escherichia coli are U-Box E3 ubiquitin ligases. PLoS pathogens 6, e1000960 (2010).
25. Tobe, T. et al. An extensive repertoire of type III secretion effectors in Escherichia coli O157 and the role of lambdoid phages in their dissemination. Proc Natl Acad Sci U S A 103, 14941-14946 (2006).
26. Ren, X. & Hurley, J. H. VHS domains of ESCRT-0 cooperate in high-avidity binding to polyubiquitinated cargo. EMBO J 29, 1045-1054 (2010).
27. Oldham, C. E., Mohney, R. P., Miller, S. L., Hanes, R. N. & O'Bryan, J. P. The ubiquitin-interacting motifs target the endocytic adaptor protein epsin for ubiquitination. Curr Biol 12, 1112-1116 (2002).
28. Polo, S. et al. A single motif responsible for ubiquitin recognition and monoubiquitination in endocytic proteins. Nature 416, 451-455 (2002).
29. Hirano, S. et al. Double-sided ubiquitin binding of Hrs-UIM in endosomal protein sorting. Nat Struct Mol Biol 13, 272-277 (2006).
30. Prag, G. et al. The Vps27/Hse1 complex is a GAT domain-based scaffold for ubiquitin-dependent sorting. Dev Cell 12, 973-986 (2007).
31. Hoeller, D. et al. E3-independent monoubiquitination of ubiquitin-binding proteins. Mol Cell 26, 891-898 (2007).
32. Yang, Y. et al. Inhibitors of ubiquitin-activating enzyme (E1), a new class of potential cancer therapeutics. Cancer research 67, 9472-9481 (2007).
33. Keren-Kaplan, T. et al. Structure-based in silico identification of ubiquitin-binding domains provides insights into the ALIX-V:ubiquitin complex and retrovirus budding. EMBO J 32, 538-551 (2013).
34. Misra, S., Beach, B. M. & Hurley. J. H. Structure of the VHS domain of human Tom1 (target of myb 1): insights into interactions with proteins and membranes. Biochemistry 39, 11282-11290 (2000).
35. Lange, A. et al. Evidence for cooperative and domain-specific binding of the signal transducing adaptor molecule 2 (STAM2) to Lys63-linked diubiquitin. J Biol Chem 287, 18687-18699 (2012).
36. Hong, Y. H. et al. Identification of a novel ubiquitin binding site of STAM1 VHS domain by NMR spectroscopy. FEBS letters 583, 287-292 (2009).
37. Ford, M. G. et al. Curvature of clathrin-coated pits driven by epsin. Nature 419, 361-366 (2002).
38. Misra, S., Puertollano, R., Kato, Y., Bonifacino, J. S. & Hurley, J. H. Structural basis for acidic-cluster-dileucine sorting-signal recognition by VHS domains. Nature 415, 933-937 (2002).
39. Shiba, T. et al. Structural basis for recognition of acidic-cluster dileucine sequence by GGA1. Nature 415, 937-941 (2002).
40. Levin-Reisman, I. et al. Automated imaging with ScanLag reveals previously undetectable bacterial growth phenotypes. Nat Methods 7, 737-739 (2010).
41. Schindelin, J. et al. Fiji: an open-source platform for biological-image analysis. Nat Methods 9, 676-682 (2012).
42. Vaynberg, J. et al. Structure of an ultraweak protein-protein complex and its crucial role in regulation of cell morphology and motility. Mol Cell 17, 513-523 (2005).
43. Malovannaya. A. et al. Analysis of the human endogenous coregulator complexome. Cell 145, 787-799 (2011).
44. Huang, F. et al. Lysine 63-linked polyubiquitination is required for EGF receptor degradation. Proc Natl Acad Sci U S A 110, 15722-15727 (2013).
45. Vina-Vilaseca, A. & Sorkin, A. Lysine 63-linked Polyubiquitination of the Dopamine Transporter Requires WW3 and WW4 Domains of Nedd4-2 and UBE2D Ubiquitin-conjugating Enzymes. Journal of Biological Chemistry 285, 7645-7656 (2010).
46. MacGurn, J. A., Hsu, P. C. & Emr, S. D. Ubiquitin and membrane protein turnover: from cradle to grave. Annu Rev Biochem 81, 231-259 (2012).
47. Rahighi, S. & Dikic, I. Selectivity of the ubiquitin-binding modules. FEBS letters 586, 2705-2710 (2012).
48. Gibson, D. G. et al. Enzymatic assembly of DNA molecules up to several hundred kilobases. Nat Methods 6, 343-345 (2009).
49. Crackower, M. A. et al. Characterization of the split hand/split foot malformation locus SHFM1 at 7q21.3-q22.1 and analysis of a candidate gene for its expression during limb development. Human molecular genetics 5, 571-579 (1996).
50. Funakoshi, M., Li, X., Velichutina, I., Hochstrasser, M. & Kobayashi, H. Sem1, the yeast ortholog of a human BRCA2-binding protein. is a component of the proteasome regulatory particle that enhances proteasome stability. J Cell Sci 117, 6447-6454 (2004).
51. Jantti, J., Lahdenranta, J., Olkkonen, V. M., Soderlund, H. & Keranen, S. SEM1, a homologue of the split hand/split foot malformation candidate gene Dss1. regulates exocytosis and pseudohyphal differentiation in yeast. Proc Natl Acad Sci U S A 96. 909-914 (1999).
52. Marston, N. J. et al. Interaction between the product of the breast cancer susceptibility gene BRCA2 and DSS1, a protein functionally conserved from yeast to mammals. Mol Cell Biol 19, 4633-4642 (1999).
53. Ma, Y. Y. et al. Identification of the deleted in split hand/split foot 1 protein as a novel biomarker for human cervical cancer.
54. Honda, R., Tanaka, H. & Yasuda, H. Oncoprotein MDM2 is a ubiquitin ligase E3 for tumor suppressor p53. FEBS letters 420, 25-27 (1997).
55. Chatterjee, C., McGinty, R. K., Fierz, B. & Muir, T. W. Disulfide-directed histone ubiquity lation reveals plasticity in hDot1L activation. Nat Chem Biol 6, 267-269 (2010).
56. Chen, J., Ai, Y., Wang, J., Haracska, L. & Zhuang, Z. Chemically ubiquitylated PCNA as a probe for eukaryotic translesion DNA synthesis. Nat Chem Biol 6, 270-272 (2010).
57. Kumar, K. S., Spasser, L., Erlich, L. A., Bavikar, S. N. & Brik, A. Total Chemical Synthesis of Di-ubiquitin Chains. Angew Chem Int Ed Engl 49, 9126-9131 (2010).
58. Kamadurai, H. B. et al. Mechanism of ubiquitin ligation and lysine prioritization by a HECT E3. eLife 2. e00828 (2013).
59. Du, Y., Xu, N., Lu, M. & Li, T. hUbiquitome: a database of experimentally verified ubiquitination cascades in humans. Database: the journal of biological databases and curation 2011, bar055 (2011).
60. Scott, D. C., Monda, J. K., Bennett, E. J., Harper, J. W. & Schulman, B. A. N-terminal acetylation acts as an avidity enhancer within an interconnected multiprotein complex. Science 334, 674-678 (2011).
61. Otwinowski, Z. & Minor, W. Processing of X-ray diffraction data collected in oscillation mode. Methods Enzymology 176, 307-326 (1997).
62. McCoy, A. J. Solving structures of protein complexes by molecular replacement with Phaser. Acta crystallographica. Section D, Biological crystallography 63, 32-41 (2007).
63. Adams, P. D. et al. PHENIX: building new software for automated crystallographic structure determination. Acta crystallographica. Section D, Biological crystallography 58, 1948-1954 (2002).
64. Murshudov, G. N., Vagin, A. A. & Dodson, E. J. Refinement of macromolecular structures by the maximum-likelihood method. Acta crystallographica. Section D, Biological crystallography 53, 240-255 (1997).
65. Emsley, P. & Cowtan, K. Coot: model-building tools for molecular graphics. Acta crystallographica. Section D, Biological crystallography 60, 2126-2132 (2004).
66. Mizuno, E., Kawahata, K., Kato, M., Kitamura, N. & Komada, M. STAM proteins bind ubiquitinated proteins on the early endosome via the VHS domain and ubiquitin-interacting motif. Mol Biol Cell 14. 3675-3689 (2003).
67. Adhya, S. L., Court, D. L., Friedman, D. I. & Gottesman, M. E. Obituary-Amos Oppenheim (31 Oct. 1934 to 24 Sep. 2006). Molecular Microbiology 67, 685-686 (2008).

What is claimed is:

1. A composition comprising a first and a second expression vector:
wherein the first expression vector comprises a first origin of replication, a first promoter, a polynucleotide capable of expressing at least one enzyme that is part of a ubiquitin pathway, a polynucleotide encoding a first antibiotic resistance protein, and a polynucleotide encoding a first fragment of a second antibiotic resistance protein;
wherein the second expression vector comprises, a second origin of replication, a second promoter, a polynucleotide capable of expressing a ubiquitinating enzyme target substrate, a polynucleotide encoding a third antibiotic resistance gene, and a polynucleotide encoding a second fragment of the second antibiotic resistance protein;
wherein the first, second, and third antibiotic resistance proteins are orthogonal to each other; and
wherein the first and the second origin of replications are not the same.

2. The composition of claim 1, wherein the first and second expression vectors are expressed in a bacterium.

3. The composition of claim 1, further comprising at least a third expression vector.

4. The composition of claim 1, wherein:
the first antibiotic resistance protein confers resistance to kanamycin; and
the third antibiotic resistance protein confers resistance to streptomycin.

5. The composition of claim 1, wherein:
the second antibiotic resistance protein confers resistance to trimethoprim.

6. The composition of claim 5, wherein the second antibiotic resistance protein is a dihydrofolate reductase (DHFR) protein.

7. The composition of claim 6,
wherein the 3′ end of the polynucleotide that encodes the first fragment of the second antibiotic resistance protein is linked to the 5′ end of the nucleotide that encodes the enzyme that is part of the ubiquitin pathway; and
wherein the 3′ end of the polynucleotide that encodes the second fragment of the second antibiotic resistance protein is linked to the 5′ end of the nucleotide that encodes the ubiquitinating enzyme target substrate.

8. The composition of claim 7, wherein the polynucleotide that encodes the first fragment of the second antibiotic resistance protein encodes the N-terminal fragment of the DHFR protein and the polynucleotide that encodes the second fragment of the second antibiotic resistance protein encodes the C-terminal fragment of the DHFR protein.

9. The composition of claim 8, wherein the 3′ end of the polynucleotide encoding the N-terminal fragment of the DHFR protein is linked to the 5′ end of the polynucleotide capable of expressing the at least one enzyme that is part of the ubiquitin pathway via a linker.

10. The composition of claim 9, wherein the 3′ end of the polynucleotide encoding the C-terminal fragment of the DHFR protein is linked to the 5′ end of the ubiquitinating enzyme target substrate via a linker.

11. The composition of claim 10, wherein the N-terminal fragment of the DHFR and the C-terminal fragment of the DHFR protein together comprise the entire DHFR protein.

12. The composition of claim 11, wherein the active site of the DHFR protein is either completely part of the N-terminal fragment or part of the C-terminal fragment.

13. The composition of claim 12, wherein the at least one enzyme that is part of the ubiquitin pathway includes an E1 protein, an E2 protein, an E3 protein, and a ubiquitin protein.

14. The composition of claim 13, wherein at least one of the two expression vectors comprises a polynucleotide that encodes the E1 protein, a polynucleotide that encodes the E2 protein, a polynucleotide that encodes the E3 protein and/or a ubiquitin.

15. The composition of claim 14, wherein at least one of the E1 protein, the E2 protein, the E3 protein, and ubiquitin protein is a eukaryotic enzyme.

16. The composition of claim 14, wherein at least one of the E1 protein, the E2 protein, the E3 protein, and ubiquitin protein is a human enzyme.

17. The composition of claim 16, wherein:
a) the first origin of replication is CDF; and
b) the second origin of replication is Col1.

18. The composition of claim 17, wherein:
a) the first vector comprises a CDF origin of replication, the polynucleotide that encodes a E1 protein, the polynucleotide that encodes a E2 protein, the polynucleotide that encodes the N-terminal fragment of DHFR protein connected via a linker to the 5′ end of the polynucleotide that encodes the human ubiquitin protein, and a kanamycin resistance gene; and
b) the second vector comprises a ColE1 origin of replication, a polynucleotide that encodes a E3 protein, the polynucleotide that encodes the C-terminal fragment of the DHFR protein connected via a linker to the ubiquitinating enzyme target substrate, and a streptomycin resistant gene.

19. The composition of claim 18, wherein the ubiquitinating enzyme target substrate is a protein that is either ubiquitinated differentially or not at all in a human disease or disorder.

20. A kit comprising the composition of claim 1.


SEQUENCE LISTING (68 sequences)

SEQ ID NO: 1 - protein, 33 aa, synthetic construct
  REGION 1..33: note = synthetic peptide
CSLYDKDTLVQLVETGGAHPLSREPITESMIMR

SEQ ID NO: 2 - protein, 33 aa, synthetic construct
  REGION 1..33: note = synthetic peptide
CTLFDAAAFSRLVGEGLPHPLTREPITASIIVK

SEQ ID NO: 3 - protein, 33 aa, synthetic construct; identical to SEQ ID NO: 2
  REGION 1..33: note = synthetic peptide

SEQ ID NO: 4 - protein, 140 aa, synthetic construct
  REGION 1..140: note = full length sequence of the NleG6-3 ligase
MERRAVALERQLNGGVDFLRSVNNYFQSVMAEHRENKTSNKILMEKINSCVFGTDSNHFSCPESFLTCPITLDTPANGVFMRNSQGAEICSLYDKDTLVQLVETGGAHPLSREPITESMIMRKDECHFDSKKESFVASDA

SEQ ID NO: 5 - protein, 35 aa, synthetic construct
  REGION 1..35: note = Flexible linker amino acid sequence
LIKAAQRAREAERDLAAAVAQAAAGQAVPRAAPRQ

SEQ ID NO: 6 - protein, 29 aa, synthetic construct
  REGION 1..29: note = Flexible linker amino acid sequence
GGSAGSGSGAGSGSGGSAGSSGSSGASSG

SEQ ID NO: 7 - protein, 76 aa, synthetic construct [sequence omitted]

SEQ ID NO: 8 - protein, 75 aa, synthetic construct
  REGION 1..75: note = yeast ubiquitin amino acid sequence
MQIFVKTLTGKTITLEVESSDTIDNVKSKIQDKEGIPPDQQRLIFAGKQLEDGRLSDYNIQKESTLHLVLRLRGG

SEQ ID NO: 9 - protein, 1051 aa, Triticum aestivum
MLPRKREIVAGEVEDLQKKTRAGEGEVTREEGDAAMAGRGNEIDEDLHSRQLAVYGRETMKRLFGSNVLVSGLQGLGAEIAKNLVLAGVKSVTLHDDGNVELWDLSSNFFLSENDVGQNRAQACVQKLQELNNAVLVSALTGDLTKEHLSKFQAVVFTDISLDKAIEFDDYCHSQQPPIAFIKSEVRGLFGSVFCDFGPEFTVLDVDGEEPHTGIVASISNDNPALVSCVDDERLEFQDGDLVVFSEVHGMTELNDGKPRKVKNARPYSFFLEEDTSSFGAYVRGGIVTQVKPPKVIKFKPLKEAMSEPGEFLMSDFSKFERPPLLHLAFQALDKFRTELSRFPVAGSTDDVQRVIEYAISINDTLGDRKLEEIDKKLLHHFASGSRAVLNPMAAMFGGIVGQEVVKACSGKFHPLYQFFYFDSVESLPVDPLEPGDLKPKNSRYDAQISVFGSKLQNKLEEAKIFMVGSGALGCEFLKNLALMGISCSQNGNLTLTDDDVIEKSNLSRQFLFRDWNIGQPKSTVAATAAMVINPKLHVEALQNRASPETENVFNDAFWENLDAVVNALDNVTARMYIDSRCVYFQKPLLESGTLGAKCNTQMVIPHLTENYGASRDPPEKQAPMCTVHSFPHNIDHCLTWARSEFEGLLEKTPTEVNAFLSNPTTYISAARTAGDAQARDQLERVIECLDRDKCETFQDSITWARLKFEDYFSNRVKQLTFTFPEDSMTSSGAPFWSAPKRFPRPVEFSSSDQSQLSFILAAAILRAETFGIPIPEWAKTPNKLAAEAVDKVIVPDFQPKQGVKIVTHEKATSLSSASVDDAAVIEELIAKLEEVSKTLPSGFHMNPIQFEKDDDTNFHMDVIAGFANMRARNYSIPEVDKLKAKFIAGRIIPAIATSTAMATGLVCLELYKALAGGHKVEDYRNTFANLAIPLFSIAEPVPPKTIKHQELSWTVWDRWTVTGNITLRELLEWLKEKGLNAYSISCGTSLLYNSMFPRHKERLDRKVVDVAREVAKMEVPSYRRHLDVVVACEDDDDNDVDIPLVSVYFR

SEQ ID NO: 10 - protein, 1053 aa, Triticum aestivum
MLPSKRPSDAAAGDENGRGGDARGPGSGRRRARAAAGAVTAAPQEIDEDLHSRQLAVYGRETMRRLFASDVLVSGLNGLGAEIAKNLALAGVKSVTIHDVKTVKMWDLSGNFFLSEDDIGKNRAAACVAKLQELNNAVLISALTEELTTEHLSKFQAVVFTDIDLDKAYEFDDYCHNHQPPISFIKSEVCGLFGSVFCDFGPKFTVLDVDGEDPHTGIIASISNDNPALISCVDDERLEFQDGDLVVFSEVHGMTELNDGKPRKVKNARPFSFSIEEDTSNFGIYVKGGIVTQVKEPKVLCFKALRDAMTDPGEVLLSDFSKFERPPVLHLAFQALDKFKKDHGRCPAAGCEEDAHSFLKIAAAINEASADRKLDTIDEKLFRQFASGSRAVLNPMAAMFGGIVGQEVVKACSGKFHPLNQFFYFDSVESLPTYPLEPQDLKPSNNRYDAQVSVFGSKLQKKMEEANTFVVGSGALGCEFLKNLALMGVSCSSKGKLTITDDDIIEKSNLSRQFLFRDWNIGQAKSTVAATAASAINPSLHIDALQNRACPDTENVFHDTFWEGLDVVINALDNVNARMYMDMRCLYFQKPLLESGTLGAKCNIQMVIPHLTENYGASRDPPEKQAPMCTVHSFPHNIDHCLTWARSEFEGLLEKTPNEVNSFLSNPAQYAAAMRKAGDAQARELLERVSECLNKDRCSTFDDCISWARLKFEDYFSNRVKQLTFTFPEDAATSMGAPFWSAPKRFPRALQFSAADQSHLNFIMSASILRAESFGVAIPEWAKDTSKLADVVNKIAVPTFEPKQGVNIVTDEKASNLSSTSVDDVAVIEDLLAKLQEYAKMLLPGFQMKPIQFEKDDDTNFHMDLISGLANMRARNYSIPEVDKLKAKFIAGRIIPAIATSTAMATGLVCLELYKVIAGEHPVEDYRNTFANLALPLFSMAEPVPPKVMKHKETSWTVWDRWSVQGNLTLAELLQWFADKGLTAYSISCGTSLLYNNMFARHKDRLTKKVVDIAREVAKVDVPEYRRHLDIGVACEDEDENDVDIPLVSVYFR

SEQ ID NO: 11 - protein, 1058 aa, Homo sapiens
MSSSPLSKKRRVSGPDPKPGSNCSPAQSVLSEVPSVPTNGMAKNGSEADIDEGLYSRQLYVLGHEAMKRLQTSSVLVSGLRGLGVEIAKNIILGGVKAVTLHDQGTAQWADLSSQFYLREEDIGKNRAEVSQPRLAELNSYVPVTAYTGPLVEDFLSGFQVVVLTNTPLEDQLRVGEFCHNRGIKLVVADTRGLFGQLFCDFGEEMILTDSNGEQPLSAMVSMVTKDNPGVVTCLDEARHGFESGDFVSFSEVQGMVELNGNQPMEIKVLGPYTFSICDTSNFSDYIRGGIVSQVKVPKKISFKSLVASLAEPDFVVTDFAKFSRPAQLHIGFQALHQFCAQHGRPPRPRNEEDAAELVALAQAVNARALPAVQQNNLDEDLIRKLAYVAAGDLAPINAFIGGLAAQEVMKACSGKFMPIMQWLYFDALECLPEDKEVLTEDKCLQRQNRYDGQVAVFGSDLQEKLGKQKYFLVGAGAIGCELLKNFAMIGLGCGEGGEIIVTDMDTIEKSNLNRQFLFRPWDVTKLKSDTAAAAVRQMNPHIRVTSHQNRVGPDTERIYDDDFFQNLDGVANALDNVDARMYMDRRCVYYRKPLLESGTLGTKGNVQVVIPFLTESYSSSQDPPEKSIPICTLKNFPNAIEHTLQWARDEFEGLFKQPAENVNQYLTDPKFVERTLRLAGTQPLEVLEAVQRSLVLQRPQTWADCVTWACHHWHTQYSNNIRQLLHNFPPDQLTSSGAPFWSGPKRCPHPLTFDVNNPLHLDYVMAAANLFAQTYGLTGSQDRAAVATFLQSVQVPEFTPKSGVKIHVSDQELQSANASVDDSRLEELKATLPSPDKLPGFKMYPIDFEKDDDSNFHMDFIVAASNLRAENYDIPSADRHKSKLIAGKIIPAIATTTAAVVGLVCLELYKVVQGHRQLDSYKNGFLNLALPFFGFSEPLAAPRHQYYNQEWTLWDRFEVQGLQPNGEEMTLKQFLDYFKTEHKLEITMLSQGVSMLYSFFMPAAKLKERLDQPMTEIVSRVSKRKLGRHVRALVLELCCNDESGEDVEVPYVRYTIR

SEQ ID NO: 12 - protein, 1058 aa, Oryctolagus cuniculus
MSSSPLSKKRRVSGPDPKPGSNCSPAQSVLPQVPSAPTNGMAKNGSEADIDEGLYSRQLYVLGHEAMKRLQTSSVLVSGLRGLGVEIAKNIILGGVKAVTLHDQGTAQWADLSSQFYLREEDIGKNRAEVSQPRLAELNSYVPVTAYTGPLVEDFLSGFQVVVLTNSPLEDQLRVGEFCHSRGIKLVVADTRGLFGQLFCDFGEEMILTDSNGEQPLSTMVSMVTKDNPGVVTCLDEARHGFESGDFVSFSEVQGMTELNGNQPIEIKVLGPYTFSICDTSNFSDYIRGGIVSQVKVPKKISFKSLSASLAEPDFVMTDFAKFSRPAQLHIGFQALHKFCAQHSRPPRPRNEEDAAELVTLARAVNSKASSAVQQDSLDEDLIRNLAFVAAGDLAPINAFIGGLAAQEVMKACSGKFMPIMQWLYFDALECLPEDKESLTEDKCLPRQNRYDGQVAVFGSDLQEKLGRQKYFLVGAGAIGCELLKNFAMIGLGCGENGEIIVTDMDTIEKSNLNRQFLFRPWDVTKLKSDTAAAAVHQMNPHIRVTSHQNRVGPDTERIYDDDFFQTLDGVANALDNVDARMYMDRRCVYYRKPLLESGTLGTKGNVQVVIPFLTESYSSSQDPPEKSIPICTLKNFPNAIEHTLQWARDEFEGLFKQPAENVNQYLTDPKFVERTLRLAGTQPLEVLEAVQRSLVLQLPQSWADCVTWACHHWHTQYSNNIRQLLHNFPPDQLTSSGAPFWSGPKRCPHPLTFDVSNPLHLDYVMAAANLFAQTYGLAGSQDRAAVATLLQSVQVPEFTPKSGVKIHVSDQELQSANASVDDSRLEELKATLPSPDKLPGFKMYPIDFEKDDDSNFHMDFIVAASNLRAENYDIPPADRHKSKLIAGKIIPAIATTTAAVVGLVCLELYKVVQGHRHLDSYKNGFLNLALPFFGFSEPLAAPRHQYYNQEWTLWDRFEVQGLQPNGEEMTLKQFLDYFKTEHKLEITMLSQGVSMLYSFFMPAAKLKERLDQPMTEIVSRVSKRKLGRHVRALVLELCCNDESGEDVEVPYVRYTIR

SEQ ID NO: 13 - protein, 1058 aa, Bos taurus
MSSSPLSKKRRVSGPDPKPGSNCSPAHSVLSEVPSVPANGMAKNVSDADIDEGLYSRQLYVLGHEAMKRLQTSSVLVSGLRGLGVEIAKNIILGGVKAVTLHDQGTAQWADLSSQFYLREEDIGKNRAEVSQPRLAELNSYVPVSAYTGPLVEDFLSDFQVVVLTNSPLEDQLRVGEFCHSHGIKLVVADTRGLFGQLFCDFGEEMILTDSNGEQPLSAMVSMVTKDNPGVVTCLDEARHGFESGDFVSFSEVQGMIELNGSQPMEIKVLGPYTFSICDTSNFSDYIRGGIVSQVKVPKKISFKSLPASLAEPDFVMTDFAKYSRPAQLHIGFQALHHFCAQHGRSPRPHNEEDAAELVTIAQAVNARSLPAVQQGSLDEDLIRKLAYVAAGDLAPINAFIGGLAAQEVMKACSGKFMPIMQWLYFDALECLPEDKEALTEDKCLPRQNRYDGQVAVFGSDLQERLGKQKYFLVGAGAIGCELLKNFAMIGLGCAEDGEIVVTDMDTIEKSNLNRQFLFRPWDVTKLKSDTAAAAVRQMNPHIRVTSHQNRVGPDTERIYDDDFFQNLDGVTNALDNVDARMYMDRRCVYYRKPLLESGTLGTKGNVQVVIPFLTESYSSSQDPPEKSIPICTLKNFPNAIEHTLQWARDEFEGLFKQPAENVNQYLTDPKFVERTLRLAGTQPLEVLEAVQRSLVLQRPQTWADCVTWACHHWHTQYSNNIRQLLHNFPPDQLTSSGAPFWSGPKRCPHPLTFDVSNPLHLDYVIAAANLFAQTYGLTGSQDRAAVATLLQSVQVPEFTPKSGVKIHVSDQELQSANASVDDSRLEELKATLPSPEKLPGFKMYPIDFEKDDDTNFHMDFIVAASNLRAENYDIPPADRHKSKLIAGKIIPAIATTTAAVVGLVCLELYKVVQGHRQLNSYKNGFLNLALPFFGFSEPLAAPRHQYYNQEWTLWDRFEVQGLQPNGEEMTLKQFLDYFKTEHKLEITMLSQGVSMLYSFFMPAAKLKERLDQPMTEIVSRVSKRKLGRHVRALVLELCCNDESGEDVEVPYVRYTIR

SEQ ID NO: 14 - protein, 1024 aa, Saccharomyces cerevisiae
MSSNNSGLSAAGEIDESLYSRQLYVLGKEAMLKMQTSNVLILGLKGLGVEIAKNVVLAGVKSMTVFDPEPVQLADLSTQFFLTEKDIGQKRGDVTRAKLAELNAYVPVNVLDSLDDVTQLSQFQVVVATDTVSLEDKVKINEFCHSSGIRFISSETRGLFGNTFVDLGDEFTVLDPTGEEPRTGMVSDIEPDGTVTMLDDNRHGLEDGNFVRFSEVEGLDKLNDGTLFKVEVLGPFAFRIGSVKEYGEYKKGGIFTEVKVPRKISFKSLKQQLSNPEFVFSDFAKFDRAAQLHLGFQALHQFAVRHNGELPRTMNDEDANELIKLVTDLSVQQPEVLGEGVDVNEDLIKELSYQARGDIPGVVAFFGGLVAQEVLKACSGKFTPLKQFMYFDSLESLPDPKNFPRNEKTTQPVNSRYDNQIAVFGLDFQKKIANSKVFLVGSGAIGCEMLKNWALLGLGSGSDGYIVVTDNDSIEKSNLNRQFLFRPKDVGKNKSEVAAEAVCAMNPDLKGKINAKIDKVGPETEEIFNDSFWESLDFVTNALDNVDARTYVDRRCVFYRKPLLESGTLGTKGNTQVIIPRLTESYSSSRDPPEKSIPLCTLRSFPNKIDHTIAWAKSLFQGYFTDSAENVNMYLTQPNFVEQTLKQSGDVKGVLESISDSLSSKPHNFEDCIKWARLEFEKKFNHDIKQLLFNFPKDAKTSNGEPFWSGAKRAPTPLEFDIYNNDHFHFVVAGASLRAYNYGIKSDDSNSKPNVDEYKSVIDHMIIPEFTPNANLKIQVNDDDPDPNANAANGSDEIDQLVSSLPDPSTLAGFKLEPVDFEKDDDTNHHIEFITACSNCRAQNYFIETADRQKTKFIAGRIIPAIATTTSLVTGLVNLELYKLIDNKTDIEQYKNGFVNLALPFFGFSEPIASPKGEYNNKKYDKIWDRFDIKGDIKLSDLIEHFEKDEGLEITMLSYGVSLLYASFFPPKKLKERLNLPITQLVKLVTKKDIPAHVSTMILEICADDKEGEDVEVPFITIHL

SEQ ID NO: 15 - protein, 1080 aa, Arabidopsis thaliana
MLHKRASEANDKNDNTIIGSDLASSKKRRIDFTESSSDKSSSILASGSSRGFHGDSVVQQIDMAFGNSNRQEIDEDLHSRQLAVYGRETMRRLFASNVLISGMHGLGAEIAKNLILAGVKSVTLHDERVVELWDLSSNFVFSEDDVGKNRADASVQKLQDLNNAVVVSSLTKSLNKEDLSGFQVVVFSDISMERAIEFDDYCHSHQPPIAFVKADVRGLFGSVFCDFGPEFAVLDVDGEEPHTGIIASISNENQAFISCVDDERLEFEDGDLVVFSEVEGMTELNDGKPRKIKSTRPYSFTLDEDTTNYGTYVKGGIVTQVKQPKLLNFKPLREALKDPGDFLFSDFSKFDRPPLLHLAFQALDHFKAEAGRFPVAGSEEDAQKLISIATAINTGQGDLKVENVDQKLLRHFSFGAKAVLNPMAAMFGGIVGQEVVKACSGKFHPLFQFFYFDSVESLPSEPVDSSDFAPRNSRYDAQISVFGAKFQKKLEDAKVFTVGSGALGCEFLKNLALMGVSCGSQGKLTVTDDDIIEKSNLSRQFLFRDWNIGQAKSTVAASAAAVINPRFNIEALQNRVGAETENVFDDAFWENLTVVVNALDNVNARLYVDSRCLYFQKPLLESGTLGTKCNTQSVIPHLTENYGASRDPPEKQAPMCTVHSFPHNIDHCLTWARSEFEGLLEKTPAEVNAYLSSPVEYTNSMMSAGDAQARDTLERIVECLEKEKCETFQDCLTWARLRFEDYFVNRVKQLIYTFPEDAATSTGAPFWSAPKRFPRPLQYSSSDPSLLNFITATAILRAETFGIPIPEWTKNPKEAAEAVDRVIVPDFEPRQDAKIVTDEKATTLTTASVDDAAVIDDLIAKIDQCRHNLSPDFRMKPIQFEKDDDTNYHMDVIAGLANMRARNYSIPEVDKLKAKFIAGRIIPAIATSTAMATGLVCLELYKVLDGGHKVEAYRNTFANLALPLFSMAEPLPPKVVKHRDMAWTVWDRWVLKGNPTLREVLQWLEDKGLSAYSISCGSCLLFNSMFTRHKERMDKKVVDLARDVAKVELPPYRNHLDVVVACEDEDDNDVDIPLVSIYFR

SEQ ID NO: 16 - protein, 1058 aa, Rattus norvegicus
MSSSPLSKKRRVSGPDPKPGSNCSSAQSVLSEVSSVPTNGMAKNGSEADIDESLYSRQLYVLGHEAMKMLQTSSVLVSGLRGLGVEIAKNIILGGVKAVTLHDQGTTQWADLSSQFYLREEDIGKNRAEVSQPRLAELNSYVPVTAYTGPLVEDFLSGFQVVVLTNSPLEEQLRVGEFCHSRGIKLVVADTRGLFGQLFCDFGEEMVLTDSNGEQPLSAMVSMVTKDNPGVVTCLDEARHGFETGDFVSFSEVQGMVQLNGCQPIEIKVLGPYTFSICDTSNFSDYIRGGIVSQVKVPKKISFKSLPASLAEPDFVMTDFAKYSRPAQLHIGFQALHQFCAQHNRPPRPRNEEDATELVTLAQAVNARSPPAVQQDNVDEDLIRKLAYVAAGDLAPINAFIGGLAAQEVMKACSGKFMPIMQWLYFDALECLPEDKEALTEDKCLPRQNRYDGQVAVFGSDLQEKLGKQKYFLVGAGAIGCELLKNFAMIGLGCGEGGEVVVTDMDTIEKSNLNRQFLFRPWDVTKLKSDTAAAAVRQMNPYIQVTSHQNRVGPDTERIYDDDFFQNLDGVANALDNVDARMYMDRRCVYYRKPLLESGTLGTKGNVQVVIPFLTESYSSSQDPPEKSIPICTLKNFPNAIEHTLQWARDEFEGLFKQPAENVNQYLTDSKFVERTLRLAGTQPLEVLEAVQRSLVLQRPQTWGDCVTWACHHWHTQYCNNIRQLLHNFPPDQLTSSGAPFWSGPKRCPHPLTFDVNNTLHLDYVMAAANLFAQTYGLTGSQDRAAVASLLQSVQVPEFTPKSGVKIHVSDQELQSANASVDDSRLEELKATLPSPDKLPGFKMYPIDFEKDDDSNFHMDFIVAASNLRAENYDISPADRHKSKLIAGKIIPAIATTTAAVVGLVCLELYKVVQGHQQLDSYKNGFLNLALPFFGFSEPLAAPRHQYYNQEWTLWDRFEVQGLQPNGEEMTLKQFLDYFKTEHKLEITMLSQGVSMLYSFFMPAAKLKERLDQPMTEIVSRVSKRKLGRHVRALVLELCCNDESGEDVEVPYVRYTIR

SEQ ID NO: 17 - protein, 148 aa, Saccharomyces cerevisiae
MSSSKRIAKELSDLGRDPPASCSAGPVGDDLYHWQASIMGPSDSPYAGGVFFLSIHFPTDYPFKPPKVNFTTKIYHPNINSSGNICLDILKDQWSPALTLSKVLLSICSLLTDANPDDPLVPEIAQIYKTDKAKYEATAKEWTKKYAV

SEQ ID NO: 18 - protein, 185 aa, Arabidopsis lyrata
MSSPSKRREMDLMKLMMSDYKVEMINDGMQEFYVEFNGPKDSIYEGGVWKIRVELPDAYPYKSPSVGFITKIYHPNVDEMSGSVCLDVINQTWSPMFDLVNVFETFLPQLLLYPNPSDPLNGEAAALMMRDRPTYEQRVKEYCEKYAKPRADTEEMSSDDEMSEDEYASDCDDEDDVAIAGKLDP

SEQ ID NO: 19 - protein, 148 aa, Saccharomyces cerevisiae
MSSSKRIAKELSDLERDPPTSCSAGPVGDDLYHWQASIMGPADSPYAGGVFFLSIHFPTDYPFKPPKISFTTKIYHPNINANGNICLDILKDQWSPALTLSKVLLSICSLLTDANPDDPLVPEIAHIYKTDRPKYEATAREWTKKYAV

SEQ ID NO: 20 - protein, 170 aa, Arabidopsis thaliana
MMSDYKVEMINDGMQEFFVEFSGPKDSIYEGGVWKIRVELPDAYPYKSPSVGFITKIYHPNVDEMSGSVCLDVINQTWSPMFDLVNVFETFLPQLLLYPNPSDPLNGEAAALMMRDRPTYEQRVKEYCEKYAKPRADTEEMSSDDEMSEDEYASDGDDEDDVAIAGKLDP

SEQ ID NO: 21 - protein, 236 aa, Homo sapiens
MARPLVPSSQKALLLELKGLQEEPVEGFRVTLVDEGDLYNWEVAIFGPPNTYYEGGYFKARLKFPIDYPYSPPAFRFLTKMWHPNIYETGDVCISILHPPVDDPQSGELPSERWNPTQNVRTILLSVISLLNEPNTFSPANVDASVMYRKWKESKGKDREYTDIIRKQVLGTKVDAERDGVKVPTTLAEYCVKTKAPAPDEGSDLFYDDYYEDGEVEEEADSCFGDDEDDSGTEES

SEQ ID NO: 22 - protein, 147 aa, Homo sapiens
MALKRIHKELNDLARDPPAQCSAGPVGDDMFHWQATIMGPNDSPYQGGVFFLTIHFPTDYPFKPPKVAFTTRIYHPNINSNGSICLDILRSQWSPALTISKVLLSICSLLCDPNPDDPLVPEIARIYKTDREKYNRIAREWTQKYAM

SEQ ID NO: 23 - protein, 147 aa, Homo sapiens
MALKRINKELSDLARDPPAQCSAGPVGDDMFHWQATIMGPNDSPYQGGVFFLTIHFPTDYPFKPPKVAFTTRIYHPNINSNGSICLDILRSQWSPALTISKVLLSICSLLCDPNPDDPLVPEIARIYKTDRDKYNRISREWTQKYAM

SEQ ID NO: 24 - protein, 200 aa, Homo sapiens
MANIAVQRIKREFKEVLKSEETSKNQIKVDLVDENFTELRGEIAGPPDTPYEGGRYQLEIKIPETYPFNPPKVRFITKIWHPNISSVTGAICLDILKDQWAAAMTLRTVLLSLQALLAAAEPDDPQDAVVANQYKQNPEMFKQTARLWAHVYAGAPVSSPEYTKKIENLCAMGFDRNAVIVALSSKSWDVETATELLLSN

SEQ ID NO: 25 - protein, 428 aa, Arabidopsis thaliana
MATAFAPTKLTATVPLHGSHENRLLLPIRLAPPSSFLGSTRSLSLRRLNHSNATRRSPVVSVQEVVKEKQSTNNTSLLITKEEGLELYEDMILGRSFEDMCAQMYYRGKMFGFVHLYNGQEAVSTGFIKLLTKSDSVVSTYRDHVHALSKGVSARAVMSELFGKVTGCCRGQGGSMHMFSKEHNMLGGFAFIGEGIPVATGAAFSSKYRREVLKQDCDDVTVAFFGDGTCNNGQFFECLNMAALYKLPIIFVVENNLWAIGMSHLRATSDPEIWKKGPAFGMPGVHVDGMDVLKVREVAKEAVTRARRGEGPTLVECETYRFRGHSLADPDELRDAAEKAKYAARDPIAALKKYLIENKLAKEAELKSIEKKIDELVEEAVEFADASPQPGRSQLLENVFADPKGFGIGPDGRYRCEDPKFTEGTAQV

SEQ ID NO: 26 - protein, 235 aa, Mus musculus
MARPLVPSSQKALLLELKGLQEEPVEGFRVTLVDEGDLYNWEVAIFGPPNTYYEGGYFKARLKFPIDYPYSPPAFRFLTKMWHPNIYETGDVCISILHPPVDDPQSGELPSERWNPTQNVRTILLSVISLLNEPNTFSPANVDASVMYRKWKESKGKDREYTDIIRKQVLGTKVDAERDGVKVPTTLAEYCVKTKAPAPDEGSDLFYDDYYEDGEVEEADSCFGDEEDDSGTEES

SEQ ID NO: 27 - protein, 172 aa, Saccharomyces cerevisiae
MSTPARRRLMRDFKRMKEDAPPGVSASPLPDNVMVWNAMIIGPADTPYEDGTFRLLLEFDEEYPNKPPHVKFLSEMFHPNVYANGEICLDILQNRWTPTYDVASILTSIQSLFNDPNPASPANVEAATLFKDHKSQYVKRVKETVEKSWEDDMDDMDDDDDDDDDDDDDEAD

SEQ ID NO: 28 - protein, 200 aa, Mus musculus; identical to SEQ ID NO: 24

SEQ ID NO: 29 - protein, 153 aa, Arabidopsis thaliana
MANSNLPRRIIKETQRLLSEPAPGISASPSEDNMRYFNVMILGPTQSPYEGGVFKLELFLPEEYPMAAPKVRFLTKIYHPNIDKLGRICLDILKDKWSPALQIRTVLLSIQALLSAPNPDDPLSENIAKHWKSNEAEAVDTAKEWTRLYASGA

SEQ ID NO: 30 - protein, 295 aa, Saccharomyces cerevisiae
MSSRKSTASSLLLRQYRELTDPKKAIPSFHIELEDDSNIFTWNIGVMVLNEDSIYHGGFFKAQMRFPEDFPFSPPQFRFTPAIYHPNVYRDGRLCISILHQSGDPMTDEPDAETWSPVQTVESVLISIVSLLEDPNINSPANVDAAVDYRKNPEQYKQRVKMEVERSKQDIPKGFIMPTSESAYISQSKLDEPESNKDMADNFWYDSDLDDDENGSVILQDDDYDDGNNHIPFEDDDVYNYNDNDDDDERIEFEDDDDDDDDSIDNDSVMDRKQPHKAEDESEDVEDVERVSKKI

SEQ ID NO: 31 - protein, 145 aa, Homo sapiens
MAVSTGVKVPRNFRLLEELEEGQKGVGDGTVSWGLEDDEDMTLTRWTGMIIGPPRTNYENRIYSLKVECGPKYPEAPPSVRFVTKINMNGINNSSGMVDARSIPVLAKWQNSYSIKVVLQELRRLMMSKENMKLPQPPEGQTYNN

SEQ ID NO: 32 - protein, 215 aa, Saccharomyces cerevisiae
MSRAKRIMKEIQAVKDDPAAHITLEFVSESDIHHLKGTFLGPPGTPYEGGKFVVDIEVPMEYPFKPPKMQFDTKVYHPNISSVTGAICLDILKNAWSPVITLKSALISLQALLQSPEPNDPQDAEVAQHYLRDRESFNKTAALWTRLYASETSNGQKGNVEESDLYGIDHDLIDEFESQGFEKDKIVEVLRRLGVKSLDPNDNNTANRIIEELLK

SEQ ID NO: 33 - protein, 250 aa, Saccharomyces cerevisiae
MATKQAHKRLTKEYKLMVENPPPYILARPNEDNILEWHYIITGPADTPYKGGQYHGTLTFPSDYPYKPPAIRMITPNGRFKPNTRLCLSMSDYHPDTWNPGWSVSTILNGLLSFMTSDEATTGSITTSDHQKKTLARNSISYNTFQNVRFKLIFPEVVQENVETLEKRKLDEGDAANTGDETEDPFTKAAKEKVISLEEILDPEDRIRAEQALRQSENNSKKDGKEPNDSSSMVYIGIAIFLFLVGLFMK

SEQ ID NO: 34 - protein, 148 aa, Arabidopsis thaliana
MASKRILKELKDLQKDPPTSCSAGPVAEDMFHWQATIMGPSESPYAGGVFLVTIHFPPDYPFKPPKVAFRTKVFHPNINSNGSICLDILKEQWSPALTISKVLLSICSLLTDPNPDDPLVPEIAHMYKTDKNKYESTARSWTQKYAMG

SEQ ID NO: 35 - protein, 318 aa, Mus musculus
METRYNLKSPAVKRLMKEAAELKDPTDHYHAQPLEDNLFEWHFTVRGPPDSDFDGGVYHGRIVLPPEYPMKPPSIILLTANGRFEVGKKICLSISGHHPETWQPSWSIRTALLAIIGFMPTKGEGAIGSLDYTPEERRALAKKSQDFCCEGCGSAMKDVLLPLKSGSGSSQADQEAKELARQISFKAEVNSSGKTIAESDLNQCFSLNDSQDDLPTTFQGATASTSYGAQNPSGAPLPQPTQPAPKNTSMSPRQRRAQQQSQRRPSTSPDVLQGQPPRAHHTEHGGSAMLIIILTLALAALIFRRIYLANEYIFDFEL

SEQ ID NO: 36 - protein, 148 aa, Arabidopsis thaliana
MASKRILKELKDLQKDPPSNCSAGPVAEDMFHWQATIMGPPESPYAGGVFLVSIHFPPDYPFKPPKVSFKTKVYHPNINSNGSICLDILKEQWSPALTISKVLLSICSLLTDPNPDDPLVPEIAHMYKTDRSKYESTARSWTQKYAMG

SEQ ID NO: 37 - protein, 491 aa, Homo sapiens
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 38 - protein, 465 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 39 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 40 - protein, 809 aa, Saccharomyces cerevisiae
MPSSISVKLVAAESLYKRDVFRSPDPFAVLTIDGYQTKSTSAAKKTLNPYWNETFKFDDINENSILTIQVFDQKKFKKKDQGFLGVVNVRVGDVLGHLDEDTATSSGRPREETITRDLKKSNDGMAVSGRLIVVLSKLPSSSPHSQAPSGHTASSSTNTSSTTRTNGHSTSSTRNHSTSHPSRGTAQAVESTLQSGTTAATNTATTSHRSTNSTSSATRQYSSFEDQYGRLPPGWERRTDNFGRTYYVDHNTRTTTWKRPTLDQTEAERGNQLNANTELERRQHRGRTLPGGSSDNSSVTVQVGGGSNIPPVNGAAAAAFAATGGTTSGLGELPSGWEQRFTPEGRAYFVDHNTRTTTWVDPRRQQYIRTYGPTNTTIQQQPVSQLGPLPSGWEMRLTNTARVYFVDHNTKTTTWDDPRLPSSLDQNVPQYKRDFRRKVIYFRSQPALRILPGQCHIKVRRKNIFEDAYQEIMRQTPEDLKKRLMIKFDGEEGLDYGGVSREFFFLLSHEMFNPFYCLFEYSAYDNYTIQINPNSGINPEHLNYFKFIGRVVGLGVFHRRFLDAFFVGALYKMMLRKKVVLQDMEGVDAEVYNSLNWMLENSIDGVLDLTFSADDERFGEVVTVDLKPDGRNIEVTDGNKKEYVELYTQWRIVDRVQEQFKAFMDGFNELIPEDLVTVFDERELELLIGGIAEIDIEDWKKHTDYRGYQESDEVIQWFWKCVSEWDNEQRARLLQFTTGTSRIPVNGFKDLQGSDGPRRFTIEKAGEVQQLPKSHTCFNRVDLPQYVDYDSMKQKLTLAVEETIGFGQE

SEQ ID NO: 41 - protein, 501 aa, Homo sapiens
MAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEACSRQHRLDQDKIEALSSKVQQLERSIGLKDLAMADLEQKVLEMEASTYDGVFIWKISDFARKRQEAVAGRIPAIFSPAFYTSRYGYKMCLRIYLNGDGTGRGTHLSLFFVVMKGPNDALLRWPFNQKVTLMLLDQNNREHVIDAFRPDVTSSSFQRPVNDMNIASGCPLFCPVSKMEAKNSYVRDDAIFIKAIVDLTGL

SEQ ID NO: 42 - protein, 282 aa, Homo sapiens
MSRQTATALPTGTSKCPPSQRVPALTGTTASNNDLASLFECPVCFDYVLPPILQCQSGHLVCSNCRPKLTCCPTCRGPLGSIRNLAMEKVANSVLFPCKYASSGCEITLPHTEKADHEELCEFRPYSCPCPGASCKWQGSLDAVMPHLMHQHKSITTLQGEDIVFLATDINLPGAVDWVMMQSCFGFHFMLVLEKQEKYDGHQQFFAIVQLIGTRKQAENFAYRLELNGHRRRLTWEATPRSIHEGIATAIMNSDCLVFDTSIAQLFAENGNLGINVTISMC

SEQ ID NO: 43 - protein, 464 aa, Mus musculus
MIVFVRFNSSYGFPVEVDSDTSILQLKEVVAKRQGVPADQLRVIFAGKELPNHLTVQNCDLEQQSIVHIVQRPRRRSHETNASGGDEPQSTSEGSIWESRSLTRVDLSSHTLPVDSVGLAVILDTDSKRDSEAARGPVKPTYNSFFIYCKGPCHKVQPGKLRVQCGTCKQATLTLAQGPSCWDDVLIPNRMSGECQSPDCPGTRAEFFFKCGAHPTSDKDTSVALNLITSNRRSIPCIACTDVRSPVLVFQCNHRHVICLDCFHLYCVTRLNDRQFVHDAQLGYSLPCVAGCPNSLIKELHHFRILGEEQYTRYQQYGAEECVLQMGGVLCPRPGCGAGLLPEQGQRKVTCEGGNGLGCGFVFCRDCKEAYHEGDCDSLLEPSGATSQAYRVDKRAAEQARWEEASKETIKKTTKPCPRCNVPIEKNGGCMHMKCPQPQCKLEWCWNCGCEWNRACMGDHWFDV

SEQ ID NO: 44 - protein, 303 aa, Homo sapiens
MKGKEEKEGGARLGAGGGSPEKSPSAQELKEQGNRLFVGRKYPEAAACYGRAITRNPLVAVYYTNRALCYLKMQQHEQALADCRRALELDGQSVKAHFFLGQCQLEMESYDEAIANLQRAYSLAKEQRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 45 - protein, 1030 aa, Danio rerio
MTTGRNNRVMMEGVGARVIRGPDWKWGKQDGGEGHVGTVRSFESPEEVVVVWDNGTAANYRCSGAYDVRILDSAPTGIKHDGTMCDTCRQQPIIGIRWKCAECTNYDLCTTCYHGDKHHLRHRFYRITTPGSERVLLESRRKSKKITARGIFAGGRVVRGVDWQWEDQDGGNGRRGKVTEIQDWSAASPHSAAYVLWDNGAKNLYRVGFEGMSDLKCVQDAKGGTFYRDHCPVLGEQNGNRNPGGLQIGDLVNIDLDLEIVQSLQHGHGGWTDGMFETLTTTGTVCGIDEDHDIVVQYPSGNRWTFNPAVLTKANVVRSGEVAAGAEGGSSQFMVGDLVQICYDIDRIKLLQRGHGEWAEAMLPTLGKVGRVQQIYSDSDLKVEVCGTSWTYNPAAVTKVAPAGSAVTNASGERLSQLLKKLFETQESGDINEELVKAAANGDLAKVEDILKRPDVDVNGQCAGHTAMQAASQNGHVDVLKLLLKHSVDLEAEDKDGDRAVHHASFGDEGSVIEVLHRGGADLNARNKRRQTPLHIAVNKGHLQVVKTLLDFGCHPSLQDSEGDTPLHDAISKKRDDMLSVLLEAGADVTITNNNGFNALHHAALRGNPSAMRVLLSKLPRPWIVDEKKDDGYTALHLAALNNHVEVAELLVHQGNANLDVQNVNQQTALHLAVERQHTQIVRLLVRAEAKLDVQDKDGDTPLHEALRHHTLSQLRQLQDMQDVSKVEPWEPSKNTLIMGLGTQGAEKKSAASIACFLAANGADLTIRNKKGQSPLDLCPDPSLCKALAKCHKEKTSGQVGSRSPSLNSNNETLEECMVCSDMKRDTLFGPCGHIATCSLCSPRVKKCLICKEQVQSRTKIEECVVCSDKKAAVLFQPCGHMCACENCASLMKKCVQCRAVVERRTPFVLCCGGKGMEDATDDEDLTGGSNSMAGGSQDLLQPNNLALSWSSGNIPALQRDKDNTNVNADVQKLQQQLQDIKEQTMCPVCLDRLKNMIFMCGHGTCQLCGDRMSECPICRKAIERRILLY

SEQ ID NO: 46 - protein, 757 aa, Homo sapiens
MSNPGTRRNGSSIKIRLTVLCAKNLAKKDFFRLPDPFAKIVVDGSGQCHSTDTVKNTLDPKWNQHYDLYVGKTDSITISVWNHKKIHKKQGAGFLGCVRLLSNAISRLKDTGYQRLDLCKLNPSDTDAVRGQIVVSLQTRDRIGTGGSVVDCRGLLENEGTVYEDSGPGRPLSCFMEEPAPYTDSTGAAAGGGNCRFVESPSQDQRLQAQRLRNPDVRGSLQTPQNRPHGHQSPELPEGYEQRTTVQGQVYFLHTQTGVSTWHDPRIPSPSGTIPGGDAAFLYEFLLQGHTSEPRDLNSVNCDELGPLPPGWEVRSTVSGRIYFVDHNNRTTQFTDPRLHHIMNHQCQLKEPSQPLPLPSEGSLEDEELPAQRYERDLVQKLKVLRHELSLQQPQAGHCRIEVSREEIFEESYRQIMKMRPKDLKKRLMVKFRGEEGLDYGGVAREWLYLLCHEMLNPYYGLFQYSTDNIYMLQINPDSSINPDHLSYFHFVGRIMGLAVFHGHYINGGFTVPFYKQLLGKPIQLSDLESVDPELHKSLVWILENDITPVLDHTFCVEHNAFGRILQHELKPNGRNVPVTEENKKEYVRLYVNWRFMRGIEAQFLALQKGFNELIPQHLLKPFDQKELELIIGGLDKIDLNDWKSNTRLKHCVADSNIVRWFWQAVETFDEERRARLLQFVTGSTRVPLQGFKALQGSTGAAGPRLFTIHLIDANTDNLPKAHTCFNRIDIPPYESYEKLYEKLLTAVEETCGFAVE

SEQ ID NO: 47 - protein, 1319 aa, Homo sapiens
MAQSLRLHFAARRSNTYPLSETSGDDLDSHVHMCFKRPTRISTSNVVQMKLTPRQTALAPLIKENVQSQERSSVPSSENVNKKSSCLQISLQPTRYSGYLQSSNVLADSDDASFTCILKDGIYSSAVVDNELNAVNDGHLVSSPAICSGSLSNFSTSDNGSYSSNGSDFGSCASITSGGSYTNSVISDSSSYTFPPSDDTFLGGNLPSDSTSNRSVPNRNTTPCEIFSRSTSTDPFVQDDLEHGLEIMKLPVSRNTKIPLKRYSSLVIFPRSPSTTRPTSPTSLCTLLSKGSYQTSHQFIISPSEIAHNEDGTSAKGFLSTAVNGLRLSKTICTPGEVRDIRPLHRKGSLQKKIVLSNNTPRQTVCEKSSEGYSCVSVHFTQRKAATLDCETTNGDCKPEMSEIKLNSDSEYIKLMHRTSACLPSSQNVDCQININGELERPHSQMNKNHGILRRSISLGGAYPNISCLSSLKHNCSKGGPSQLLIKFASGNEGKVDNLSRDSNRDCTNELSNSCKTRDDFLGQVDVPLYPLPTENPRLERPYTFKDFVLHPRSHKSRVKGYLRLKMTYLPKTSGSEDDNAEQAEELEPGWVVLDQPDAACHLQQQQEPSPLPPGWEERQDILGRTYYVNHESRRTQWKRPTPQDNLTDAENGNIQLQAQRAFTTRRQISEETESVDNRESSENWEIIREDEATMYSNQAFPSPPPSSNLDVPTHLAEELNARLTIFGNSAVSQPASSSNHSSRRGSLQAYTFEEQPTLPVLLPTSSGLPPGWEEKQDERGRSYYVDHNSRTTTWTKPTVQATVETSQLTSSQSSAGPQSQASTSDSGQQVTQPSEIEQGFLPKGWEVRHAPNGRPFFIDHNTKTTTWEDPRLKIPAHLRGKTSLDTSNDLGPLPPGWEERTHTDGRIFYINHNIKRTQWEDPRLENVAITGPAVPYSRDYKRKYEFFRRKLKKQNDIPNKFEMKLRRATVLEDSYRRIMGVKRADFLKARLWIEFDGEKGLDYGGVAREWFFLISKEMFNPYYGLFEYSATDNYTLQINPNSGLCNEDHLSYFKFIGRVAGMAVYHGKLLDGFFIRPFYKMMLHKPITLHDMESVDSEYYNSLRWILENDPTELDLRFIIDEELFGQTHQHELKNGGSEIVVTNKNKKEYIYLVIQWRFVNRIQKQMAAFKEGFFELIPQDLIKIFDENELELLMCGLGDVDVNDWREHTKYKNGYSANHQVIQWFWKAVLMMDSEKRIRLLQFVTGTSRVPMNGFAELYGSNGPQSFTVEQWGTPEKLPRAHTCFNRLDLPPYESFEELWDKLQMAIENTQGFDGVD

SEQ ID NO: 48 - protein, 887 aa, Mus musculus
MSSDMAADESEAPVLSEDEVWEFCLDKTEDGGGSPGSDVTDTCEPPCGCWELNPNSLEEEHVLFTADPYLELHNDDTRVVRVKVIAGIGLAKKDILGASDPYVRVTLYDPMSGILTSVQTKTIKKSLNPKWNEEILFRVLPQRHRILFEVFDENRLTRDDFLGQVDVPLYPLPTENPRMERPYTFKDFVLHPRSHKSRVKGYLRLKMTYLPKNGSEDENADQAEELEPGWVVLDQPDAATHLPHPPEPSPLPPGWEERQDVLGRTYYVNHESRRTQWKRPSPDDDLTDEDNDDMQLQAQRAFTTRRQISEDVDGPDNRESPENWEIVREDENTEYSGQAVQSPPSGHIDVQTHLAEEFNTRLAVCGNPATSQPVTSSNHSSRGGSLQTCIFEEQPTLPVLLPTSSGLPPGWEEKQDDRGRSYYVDHNSKTTTWSKPTMQDDPRSKIPAHLRGKTDSNDLGPLPPGWEERTHTDGRVFFINHNIKKTQWEDPRLQNVAITGPAVPYSRDYKRKYEFFRRKLKKQTDIPNKFEMKLRRANILEDSYRRIMGVKRADLLKARLWIEFDGEKGLDYGGVAREWFFLISKEMFNPYYGLFEYSATDNYTLQINPNSGLCNEDHLSYFKFIGRVAGMAVYHGKLLDGFFIRPFYKMMLQKLITLHDMESVDSEYYSSLRWILENDPTELDLRFIIDEELFGQTHQHELKTGGSEIVVTNKNKKEYIYLVIQWRFVNRIQKQMAAFKEGFFELIPQDLIKIFDENELELLMCGLGDVDVNDWREHTKYKNGYSMNHQVIHWFWKAVWMMDSEKRIRLLQFVTGTSRVPMNGFAELYGSNGPQSFTVEQWGTPDKLPRAHTCFNRLDLPPYESFDELWDKLQMAIENTQGFDGVD

SEQ ID NO: 49 - protein, 324 aa, Homo sapiens
MSRPSSTGPSANKPCSKQPPPQPQHTPSPAAPPAAATISAAGPGSSAVPAAAAVISGPGGGGGAGPVSPQHHELTSLFECPVCFDYVLPPILQCQAGHLVCNQCRQKLSCCPTCRGALTPSIRNLAMEKVASAVLFPCKYATTGCSLTLHHTEKPEHEDICEYRPYSCPCPGASCKWQGSLEAVMSHLMHAHKSITTLQGEDIVFLATDINLPGAVDWVMMQSCFGHHFMLVLEKQEKYEGHQQFFAIVLLIGTRKQAENFAYRLELNGNRRRLTWEATPRSIHDGVAAAIMNSDCLVFDTAIAHLFADNGNLGINVTISTCCP

SEQ ID NO: 50 - protein, 301 aa, Arabidopsis thaliana
MAFNHRKMLLSCLQFKDLRFCFRQYPPPPPPPPPPRELSLLLPTSICVVGSIILFLFLVFFLYLHITQQRRISAASVTPGDTNQQEDEDETEERDFSDFHHVWQIPTVGLHRSAINSITVVGFKKGEGIIDGTECSVCLNEFEEDESLRLLPKCSHAFHLNCIDTWLLSHKNCPLCRAPVLLITEPPHQETETNHQPDSESSNDLRGRQDSSRSRRNHNIFLPRAQSDLANYCGSGRVENVRRSFSIGGSLSLCDGINNATRSGRQFYTSFSANLFSSSRRVRNEQPIPQNQMPSVTGNTS

SEQ ID NO: 51 - protein, 1030 aa, Danio rerio; identical to SEQ ID NO: 45

SEQ ID NO: 52 - protein, 522 aa, Arabidopsis thaliana
MSVKNNSFSSAEIPDVADQPRDRFNPEATQDLQEKDETKEEKEGDEEVKHDEAEEDQEVVKPNDAEEDDDGDDAEEDEEEEVEAEEDEEAEEEEEEEEEEEEEEEDSKERSPSSISGDQSEFMEIDLGEIRKDVQCPICLGIIKKTRTVMECLHRFCRECIDKSMRLGNNECPACRKHCASRRSLRDDPKFDALIAALFTNIDSYEEEELAFHEDEMARNKQIQASIAQISQRQSEALVKRRSLGKEAAVLMRSPRIASGSRRRRNSRNMEQQNASEAHEDDDNDDNNNRGRDKDSSSDERGTEVRQKKRRKRSTSRSTQHPSSSGANKNNGNCADNDTEVYRDTKGISPGLVWNPEILAWGRGGTRSNTRHGNNTSGGSSKSVRNARVNKLVEYLRSSVDGSSVELDIHVKLVSLDTKCIPDLPQPYLCCRPTLLVKQLREFVALQIHLKTEEVELLVTRRRVGEDAAIENLPAVTPASAAASKDEMLSLEDNETLSRLKIDFISSHEQHLIIAYRKKQTE

SEQ ID NO: 53 - protein, 574 aa, Homo sapiens
MATLVVNKLGAGVDSGRQGSRGTAVVKVLECGVCEDVFSLQGDKVPRLLLCGHTVCHDCLTRLPLHGRAIRCPFDRQVTDLGDSGVWGLKKNFALLELLERLQNGPIGQYGAAEESIGISGESIIRCDEDEAHLASVYCTVCATHLCSECSQVTHSTKTLAKHRRVPLADKPHEKTMCSQHQVHAIEFVCLEEGCQTSPLMCCVCKEYGKHQGHKHSVLEPEANQIRASILDMAHCIRTFTEEISDYSRKLVGIVQHIEGGEQIVEDGIGMAHTEHVPGTAENARSCIRAYFYDLHETLCRQEEMALSVVDAHVREKLIWLRQQQEDMTILLSEVSAACLHCEKTLQQDDCRVVLAKQEITRLLETLQKQQQQFTEVADHIQLDASIPVTFTKDNRVHIGPKMEIRVVTLGLDGAGKTTILFKLKQDEFMQPIPTIGFNVETVEYKNLKFTIWDVGGKHKLRPLWKHYYLNTQAVVFVVDSSHRDRISEAHSELAKLLTEKELRDALLLIFANKQDVAGALSVEEITELLSLHKLCCGRSWYIQGCDARSGMGLYEGLDWLSRQLVAAGVLDVA

SEQ ID NO: 54 - protein, 864 aa, Mus musculus
MSDSGPQLDSMGSLTMKSQLQITVISAKLKENKKNWFGPSPYVEVTVDGQSKKTEKCNNTNSPKWKQPLTVIVTPTSKLCFRVWSHQTLKSDVLLGTAGLDIYETLKSNNMKLEEVVMTLQLVGDKEPTETMGDLSVCLDGLQVEAEVVTNGETSCSESTTQNDDGCRTRDDTRVSTNGSEDPEVAASGENKRANGNNSPSLSNGGFKPSRPPRPSRPPPPTPRRPASVNGSPSTNSDSDGSSTGSLPPTNTNVNTSTSEGATSGLIIPLTISGGSGPRPLNTVSQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQNKEFDPLGPLPPGWEKRTDSNGRVYFVNHNTRITQWEDPRSQGQLNEKPLPEGWEMRFTVDGIPYFVDHNRRATTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECGLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYTRTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQE

SEQ ID NO: 55 - protein, 353 aa, Homo sapiens
MDYKSSLIQDGNPMENLEKQLICPICLEMFTKPVVILPCQHNLCRKCANDIFQAANPYWTSRGSSVSMSGGRFRCPTCRHEVIMDRHGVYGLQRNLLVENIIDIYKQECSSRPLQKGSHPMCKEHEDEKINIYCLTCEVPTCSMCKVFGIHKACEVAPLQSVFQGQKTELNNCISMLVAGNDRVQTIITQLEDSRRVTKENSHQVKEELSQKFDTLYAILDEKKSELLQRITQEQEKKLSFIEALIQQYQEQLDKSTKLVETAIQSLDEPGGATFLLTAKQLIKSIVEASKGCQLGKTEQGFENMDFFTLDLEHIADALRAIDFGTDEEEEEFIEEEDQEEEESTEGKEEGHQ

SEQ ID NO: 56 - protein, 870 aa, Homo sapiens
MASASSSRAGVALPFEKSQLTLKVVSAKPKVHNRQPRINSYVEVAVDGLPSETKKTGKRIGSSELLWNEIIILNVTAQSHLDLKVWSCHTLRNELLGTASVNLSNVLKNNGGKMENMQLTLNLQTENKGSVVSGGELTIFLDGPTVDLGNVPNGSALTDGSQLPSRDSSGTAVAPENRHQPPSTNCFGGRSRTHRHSGASARTTPATGEQSPGARSRHRQPVKNSGHSGLANGTVNDEPTTATDPEEPSVVGVTSPPAAPLSVTPNPNTTSLPAPATPAEGEEPSTSGTQQLPAAAQAPDALPAGWEQRELPNGRVYYVDHNTKTTTWERPLPPGWEKRTDPRGRFYYVDHNTRTTTWQRPTAEYVRNYEQWQSQRNQLQGAMQHFSQRFLYQSSSASTDHDPLGPLPPGWEKRQDNGRVYYVNHNTRTTQWEDPRTQGMIQEPALPPGWEMKYTSEGVRYFVDHNTRTTTFKDPRPGFESGTKQGSPGAYDRSFRWKYHQFRFLCHSNALPSHVKISVSRQTLFEDSFQQIMNMKPYDLRRRLYIIMRGEEGLDYGGIAREWFFLLSHEVLNPMYCLFEYAGKNNYCLQINPASSINPDHLTYFRFIGRFIAMALYHGKFIDTGFTLPFYKRMLNKRPTLKDLESIDPEFYNSIVWIKENNLEECGLELYFIQDMEILGKVTTHELKEGGESIRVTEENKEEYIMLLTDWRFTRGVEEQTKAFLDGFNEVAPLEWLRYFDEKELELMLCGMQEIDMSDWQKSTIYRHYTKNSKQIQWFWQVVKEMDNEKRIRLLQFVTGTCRLPVGGFAELIGSNGPQKFCIDKVGKETWLPRSHTCFNRLDLPPYKSYEQLREKLLYAIEETEGFGQE

SEQ ID NO: 57 - protein, 180 aa, Homo sapiens
MAAAEEEDGGPEGPNRERGGAGATFECNICLETAREAVVSVCGHLYCWPCLHQWLETRPERQECPVCKAGISREKVVPLYGRGSQKPQDPRLKTPPRPQGQRPAPESRGGFQPFGDTGGFHFSFGVGAFPFGFFTTVFNAHEPFRRGTGVDLGQGHPASSWQDSLFLFLAIFFFFWLLSI

SEQ ID NO: 58 - protein, 875 aa, Homo sapiens
MEKLHQCYWKSGEPQSDDIEASRMKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 59 - protein, 1068 aa, Homo sapiens
MFTLSQTSRAWFIDRARQAREERLVQKERERAAVVIQAHVRSFLCRSRLQRDIRREIDDFFKADDPESTKRSALCIFKIARKLLFLFRIKEDNERFEKLCRSILSSMDAENEPKVWYVSLACSKDLTLLWIQQIKNILWYCCDFLKQLKPEILQDSRLITLYLTMLVTFTDTSTWKILRGKGESLRPAMNHICANIMGHLNQHGFYSVLQILLTRGLARPRPCLSKGTLTAAFSLALRPVIAAQFSDNLIRPFLIHIMSVPALVTHLSTVTPERLTVLESHDMLRKFIIFLRDQDRCRDVCESLEGCHTLCLMGNLLHLGSLSPRVLEEETDGFVSLLTQTLCYCRKYVSQKKSNLTHWHPVLGWFSQSVDYGLNESMHLITKQLQFLWGVPLIRIFFCDILSKKLLESQEPAHAQPASPQNVLPVKSLLKRAFQKSASVRNILRPVGGKRVDSAEVQKVCNICVLYQTSLTTLTQIRLQILTGLTYLDDLLPKLWAFICELGPHGGLKLFLECLNNDTEESKQLLAMLMLFCDCSRHLITILDDIEVYEEQISFKLEELVTISSFLNSFVFKMIWDGIVENAKGETLELFQSVHGWLMVLYERDCRRRFTPEDHWLRKDLKPSVLFQELDRDRKRAQLILQYIPHVIPHKNRVLLFRTMVTKEKEKLGLVETSSASPHVTHITIRRSRMLEDGYEQLRQLSQHAMKGVIRVKFVNDLGVDEAGIDQDGVFKEFLEEIIKRVFDPALNLFKTTSGDERLYPSPTSYIHENYLQLFEFVGKMLGKAVYEGIVVDVPFASFFLSQLLGHHHSVFYSSVDELPSLDSEFYKNLTSIKRYDGDITDLGLTLSYDEDVMGQLVCHELIPGGKTIPVTNENKISYIHLMAHFRMHTQIKNQTAALISGFRSIIKPEWIRMFSTPELQRLISGDNAEIDLEDLKKHTVYYGGFHGSHRVIIWLWDILASDFTPDERAMFLKFVTSCSRPPLLGFAYLKPPFSIRCVEVSDDQDTGDTLGSVLRGFFTIRKREPGGRLPTSSTCFNLLKLPNYSKKSVLREKLRYAISMNTGFELS

SEQ ID NO: 60 - protein, 216 aa, Escherichia coli
MPFSFNLSSGNYLSTQDVEVLQRATRDHQMERLTIGERSFSVRYQSAMDAFIVDPVQGELYSGLSHTELADIIRLADSVENQLNGGNSFLDVFSTYMGQVISEFMHSNDNRIELLQRRLHSCSFLVNIEEMSYIDEALQCPITLAIPQRGVFLRNAEGSRVCSLYDEMALSRIINDGMHHPLSREPITLSMLVAREQCEFDCSIGHFTVRSDCYSV

SEQ ID NO: 61 - protein, 140 aa, Escherichia coli; identical to SEQ ID NO: 4

SEQ ID NO: 62 - protein, 123 aa, Escherichia coli
MLQLSSNIGWKKGAENALKNKIHSHSFVVNPDEFSCDTQFLKCPITLCVPEKGVFVKNALNSNICTLYDKSAFMNLTREHLPHPLSREKIVKEMIIERNMCYFDTISQHFIIMDADQQKQHCK

SEQ ID NO: 63 - DNA, 660 bp, synthetic construct
  misc_feature 1..660: note = An exemplary DNA sequence that encodes full length CAT
  source 1..660: mol_type = other DNA
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaa

SEQ ID NO: 64 - protein, 219 aa, synthetic construct
  REGION 1..219: note = amino acid sequence of CAT
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCDEWQGGA

SEQ ID NO: 65 - DNA, 93 bp, synthetic construct
  misc_feature 1..93: note = CAT N-terminal fragment nucleic acid sequence
  source 1..93: mol_type = other DNA
atggagaaaaaaatcactggatataccaccgttgatatatcccaatggcatcgtaaagaacattttgaggcatttcagtcagttgctcaataa

SEQ ID NO: 66 - DNA, 573 bp, synthetic construct
  misc_feature 1..573: note = CAT C-terminal fragment nucleic acid sequence
  source 1..573: mol_type = other DNA
atgtgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaa

SEQ ID NO: 67 - protein, 30 aa, synthetic construct
  REGION 1..30: note = CAT N-terminal fragment amino acid sequence
MEKKITGYTTVDISQWHRKEHFEAFQSVAQ

SEQ ID NO: 68 - protein, 190 aa, synthetic construct
  REGION 1..190: note = CAT C-terminal fragment amino acid sequence
MCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCDEWQGGA